(19) United States
(12) Patent Application Publication
(10) Pub. No.: US 2026/0356957 A1
(43) Pub. Date: Nov. 12, 2026

(54) METHODS FOR TREATMENT OF NON-SMALL CELL LUNG CANCER (NSCLC)

(71) Applicant: Janssen Biotech, Inc., Horsham, PA (US)

(72) Inventor: Roland KNOBLAUCH, Spring House, PA (US)

(21) Appl. No.: 19/485,772

(22) PCT Filed: May 23, 2024
(86) PCT No.: PCT/IB2024/055039

§ 371 Date: Nov. 19, 2025

Related U.S. Application Data
(60) Provisional application No. 63/468,375, filed on May 23, 2023.
(60) Provisional application No. 63/540,742, filed on Sep. 27, 2023.

(51) Int. Cl. C07K 16/28; A61K 9/00; A61K 31/5377; A61K 39/00; A61P 35/04

(52) U.S. Cl. CPC C07K 16/2863; A61K 9/0019; A61K 9/0053; A61K 31/5377; A61P 35/04; A61K 2039/54; A61K 2039/545; C07K 2317/31; C07K 2317/41; C07K 2317/565

(57) ABSTRACT

The present disclosure provides methods for improving median progression free survival (PFS) and improving overall survival for treatment naïve subjects or a population of treatment naïve subjects with EGFR-positive non-small cell lung cancer (NSCLC).

### CROSS-REFERENCE TO RELATED APPLICATIONS

This application claims priority to U.S. Provisional Application No. 63/540,742, filed on Sep. 27, 2023, and U.S. Provisional Application No. 63/468,375, filed on May 23, 2023, the disclosures of each of which are herein incorporated by reference in their entireties.

### SEQUENCE LISTING

The instant application contains a Sequence Listing which has been submitted electronically in XML format and is hereby incorporated by reference in its entirety. The XML copy, created on May 21, 2024, is named 103693007299_SequenceListing.xml and is 20,173 bytes in size.

### FIELD

The present disclosure provides methods for treating EGFR-positive non-small cell lung cancer (NSCLC) in a subject that is treatment naïve.

### BACKGROUND

Worldwide, lung cancer is one of the most common cancers, with NSCLC making up 80 to 85 percent of all lung cancer cases. The main subtypes of NSCLC are adenocarcinoma, squamous cell carcinoma and large cell carcinoma. Among the most common driver mutations in NSCLC are alterations in EGFR, which is a receptor tyrosine kinase controlling cell growth and division. EGFR mutations are present in 10 to 15 percent of Western patients with NSCLC with adenocarcinoma histology and occur in 40 to 50 percent of Asian patients. EGFR ex19del or EGFR exon 21 L858R mutations are the most common EGFR mutations.

In NSCLC, specific mutations in the EGFR gene are associated with high response rates to EGFR tyrosine kinase inhibitors (EGFR-TKIs). Although the majority of NSCLC patients with EGFR mutations initially respond to EGFR TKI therapy, virtually all acquire resistance that prevents a durable response. Nearly 60% of all tumors that become resistant to EGFR tyrosine kinase inhibitors increase c-Met expression, amplify the c-Met gene, or increase its only known ligand, Hepatocyte Growth Factor (Turke et al., Cancer Cell, 17:77-88, 2010). The five-year survival rate for all people with advanced NSCLC and EGFR mutations treated with EGFR TKIs is less than 20 percent. Patients with EGFR ex19del or exon 21 L858R mutations have a real-world five-year overall survival of 19 percent.

Progression of acquired resistance to EGFR-TKI such as osimertinib in epidermal growth factor receptor mutant (EGFRm) NSCLC likely arises from complex and heterogenous patterns of resistance together with co-occurrence of multiple resistance mechanisms, and as such the details of such mechanisms remain elusive. Thus, targeted therapies pose unique challenges, and there remains a need for new treatment paradigms for patients that are newly diagnosed with NSCLC.

### SUMMARY

In one aspect, provided herein is a method of improving median progression free survival (PFS) in a population of subjects with locally advanced or metastatic non-small cell lung cancer (NSCLC) harboring one or more epidermal growth factor receptor (EGFR) mutations who are treatment-naïve, the method comprising administering to the subject or the population of subjects a combination therapy comprising: (i) a therapeutically effective amount of a bispecific anti-EGFR/c-Met antibody, and (ii) a therapeutically effective amount of lazertinib, or a pharmaceutically acceptable salt or hydrate thereof, and wherein the improvement in median PFS is relative to median PFS of a reference population of subjects with NSCLC harboring one or more EGFR mutations who are treatment-naive, the reference population of subjects having been administered osimertinib or lazertinib without the bispecific anti-EGFR/c-Met antibody.

In another aspect, provided herein is a method of improving overall survival (OS) in a subject or a population of subjects with locally advanced or metastatic non-small cell lung cancer (NSCLC) harboring one or more epidermal growth factor receptor (EGFR) mutations who are treatment-naïve, the method comprising administering to the subject or a population of subjects a combination therapy comprising: (i) a therapeutically effective amount of a bispecific anti-EGFR/c-Met antibody, and (ii) a therapeutically effective amount of lazertinib, or a pharmaceutically acceptable salt or hydrate thereof, and wherein the improvement in OS is relative to OS of a reference subject or a reference population of subjects with NSCLC harboring one or more EGFR mutations who are treatment-naive, the reference subject or the reference population of subjects having been administered osimertinib or lazertinib without the bispecific anti-EGFR/c-Met antibody.

In some embodiments, the lazertinib, or pharmaceutically acceptable salt or hydrate thereof, is lazertinib mesylate. In some embodiments, the lazertinib, or pharmaceutically acceptable salt or hydrate thereof, is lazertinib mesylate monohydrate.

In some embodiments, the one or more EGFR mutations comprise one or more exon 19 deletions, or exon 21 L858R substitution, or any combination thereof. In some embodiments, the one or more EGFR mutations comprise one or more exon 19 deletions. In some embodiments, the one or more EGFR mutations comprise exon 21 L858R substitution.

In some embodiments, the subject has newly diagnosed, locally advanced or metastatic NSCLC that is not amenable to curative therapy including surgical resection or chemoradiation. In some embodiments, the curative therapy includes surgical resection or chemoradiation.

In some embodiments, the method comprises administering the lazertinib, or pharmaceutically acceptable salt or hydrate thereof, in an amount of about 80 mg to about 320 mg orally once daily. In some embodiments, the method comprises administering the lazertinib, or pharmaceutically acceptable salt or hydrate thereof, in an amount of about 240 mg orally once daily.

In some embodiments, the method elicits a clinical response in the subject according to RECIST v1.1 criteria. In some embodiments, the method achieves a partial response or better in the subject according to RECIST v1.1 criteria. In some embodiments, the clinical response comprises a median duration of response (DOR) of at least 25 months.

In some embodiments, the subject is progression-free after at least 11 months. In some embodiments, the subject is progression-free after at least 23 months. In some embodiments, the method achieves a PFS rate of 87% at 6 months, 73% at 12 months, 60% at 18 months, 48% at 24 months and 41% at 30 months in a population of the treatment naïve subjects diagnosed with locally advanced or metastatic NSCLC harboring one or more epidermal growth factor receptor (EGFR) mutations.

In some embodiments, the bispecific anti-EGFR/c-Met antibody comprises a first domain that specifically binds EGFR and a second domain that specifically binds c-Met, wherein the first domain comprises a heavy chain complementarity determining region 1 (HCDR1) comprising SEQ ID NO: 1, a HCDR2 comprising SEQ ID NO: 2, a HCDR3 comprising SEQ ID NO: 3, a light chain complementarity determining region 1 (LCDR1) comprising SEQ ID NO: 4, a LCDR2 comprising SEQ ID NO: 5, and a LCDR3 comprising SEQ ID NO: 6, and wherein the second domain that binds c-Met comprises a HCDR1 comprising SEQ ID NO: 7, a HCDR2 comprising SEQ ID NO: 8, a HCDR3 comprising SEQ ID NO: 9, a LCDR1 comprising SEQ ID NO: 10, a LCDR2 comprising SEQ ID NO: 11, and a LCDR3 comprising SEQ ID NO: 12. In some embodiments, the first domain that specifically binds EGFR comprises a heavy chain variable region (VH) comprising SEQ ID NO: 13 and a light chain variable region (VL) comprising SEQ ID NO: 14, and the second domain that specifically binds c-Met comprises the VH comprising SEQ ID NO: 15 and a VL comprising SEQ ID NO: 16. In some embodiments, the bispecific anti-EGFR/c-Met antibody is an IgG1 isotype. In some embodiments, the bispecific anti-EGFR/c-Met antibody comprises a first heavy chain (HC1) comprising SEQ ID NO: 17, a first light chain (LC1) comprising SEQ ID NO: 18, a second heavy chain (HC2) comprising SEQ ID NO: 19, and a second light chain (LC2) comprising SEQ ID NO: 20. In some embodiments, the bispecific anti-EGFR/c-Met antibody comprises a biantennary glycan structure with a fucose content of about between 1% to about 15%.

In some embodiments, the bispecific anti-EGFR/c-Met antibody is administered intravenously to the subject. In some embodiments, the bispecific anti-EGFR/c-Met antibody is administered at a dose of between about 140 mg to about 2240 mg. In some embodiments, the bispecific anti-EGFR/c-Met antibody is administered at a dose of about 700 mg, about 750 mg, about 800 mg, about 850 mg, about 900 mg, about 950 mg, about 1000 mg, about 1050 mg, about 1100 mg, about 1150 mg, about 1200 mg, about 1250 mg, about 1300 mg, about 1350 mg, about 1400 mg, about 1575 mg, about 1600 mg, about 2100 mg, or about 2240 mg. In some embodiments, the bispecific anti-EGFR/c-Met antibody is administered at a dose of 1050 mg if the subject has a body weight of less than 80 kg. In some embodiments, the bispecific anti-EGFR/c-Met antibody is administered at a dose of 1400 mg if the subject has a body weight of greater than or equal to 80 kg.

In some embodiments, the bispecific anti-EGFR/c-Met antibody is administered subcutaneously or intradermally to the subject. In some embodiments, the bispecific anti-EGFR/c-Met antibody is administered subcutaneously or intradermally at a dose sufficient to achieve a therapeutic effect in the subject.

In some embodiments, the bispecific anti-EGFR/c-Met antibody is administered twice a week, once a week, once in two weeks, once in three weeks or once in four weeks.

In some embodiments, the subject or population of subjects has a baseline brain metastasis, baseline liver metastasis, TP53 co-mutation, detectable baseline EGFRm ctDNA, or is without EGFRm ctDNA clearance at C3D1.

### BRIEF DESCRIPTION OF THE DRAWINGS

FIG. 1 shows an exemplary schematic overview of the MARIPOSA clinical study.

FIG. 2 shows Kaplan-Meier estimates of progression-free survival as assessed by blinded independent central review in the efficacy population of amivantamab-lazertinib versus osimertinib including the lazertinib monotherapy arm. PFS is shown by BICR results. The dashed lines indicate the median progression-free survival in the two groups and the tick marks indicate censoring of data. The median progression-free survival in the lazertinib group was 18.5 months (95% CI, 14.8 to 20.1).

FIG. 3 shows Kaplan-Meier estimates of progression-free survival as assessed by blinded independent central review in the efficacy population of amivantamab-lazertinib versus Osimertinib. The dashed lines indicate the median progression-free survival in the two groups and the tick marks indicate censoring of data. PFS by BICR is prolonged by 7.1 months for Amivantamab+Lazertinib vs Osimertinib.

FIG. 4 shows PFS by subgroup.

FIG. 5 shows a Kaplan-Meier estimate of interim overall survival. The efficacy population included all patients who had undergone randomization. Tick marks indicate censoring of data.

FIG. 6 shows a Consolidated Standards of Reporting Trials (CONSORT) diagram of patient disposition. 1375 patients were screened and 1074 underwent randomization (429 to amivantamab-lazertinib, 429 to osimertinib monotherapy, and 216 to lazertinib monotherapy); 1062 received at least one dose of trial treatment.

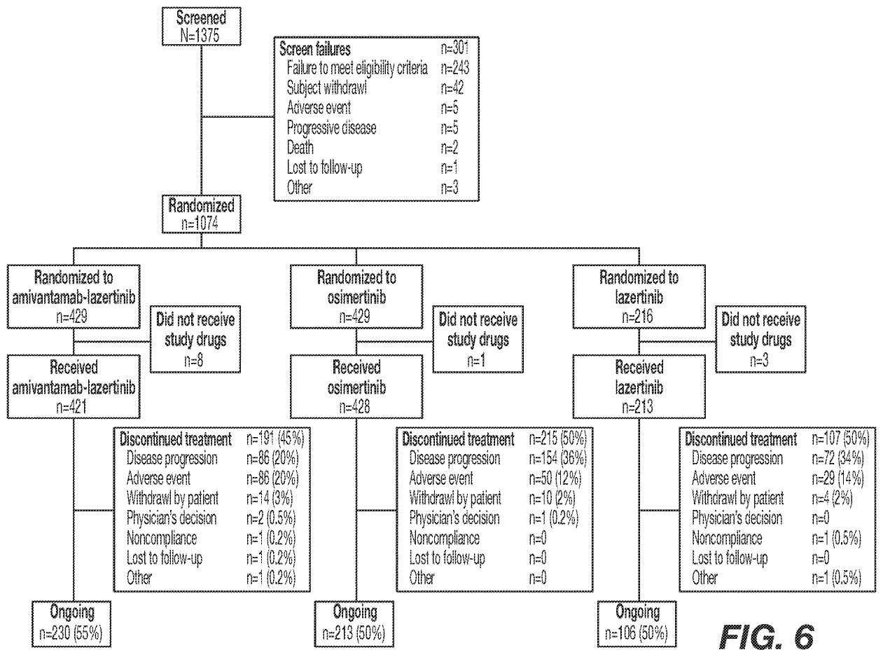

FIG. 7 shows Kaplan-Meier estimates of progression-free survival as assessed by blinded independent central review in patients with EGFR Exon 19 deletions (top) and in patients with EGFR Exon 21 L858R (bottom). The efficacy population included all patients who had undergone randomization. In both panels, the hazard ratio for disease progression or death was obtained is from an unstratified proportional hazards model; tick marks indicate censoring of data. 95% confidence interval (CI) widths have not been adjusted for multiplicity and cannot be used to infer definitive treatment effects. EGFR denotes epidermal growth factor receptor.

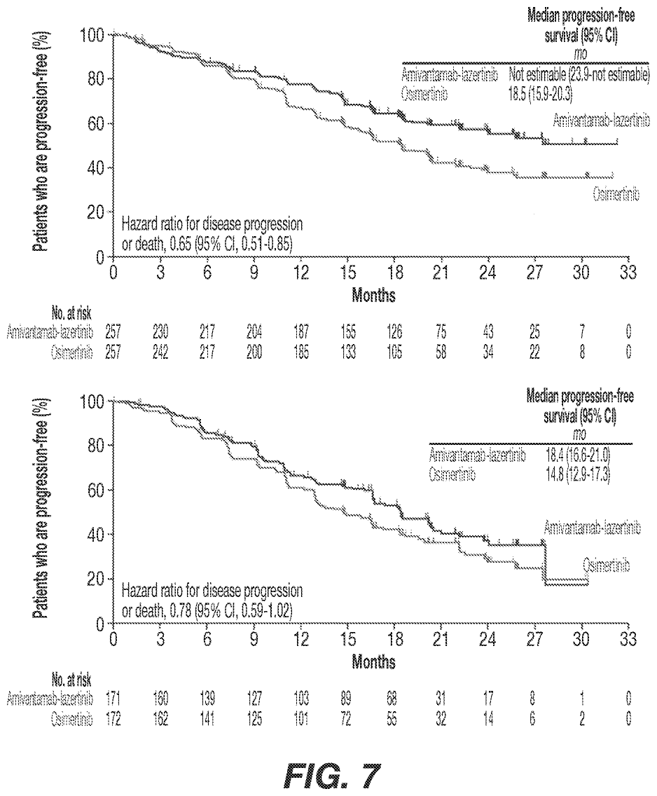

FIG. 8 shows Kaplan-Meier estimates of progression-free survival as assessed by blinded independent central review in patients who are of Asian race (top) and in patients who are of non-Asian race (bottom). The efficacy population included all patients who had undergone randomization. In both panels, the hazard ratio (HR) for disease progression or death was obtained is from an unstratified proportional hazards model; tick marks indicate censoring of data. 95% CI widths have not been adjusted for multiplicity and cannot be used to infer definitive treatment effects.

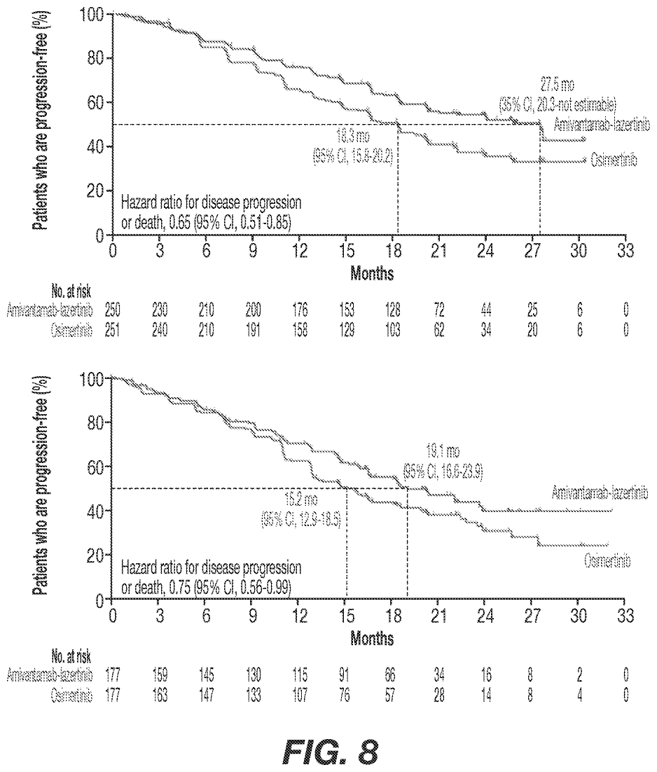

FIG. 9 shows Kaplan-Meier estimates of progression-free survival as assessed by blinded independent central review in patients with a history of brain metastases (top) and in patients without history of brain metastases (bottom). The efficacy population included all patients who had undergone randomization. In both panels, the hazard ratio for disease progression or death was obtained from an unstratified proportional hazards model; tick marks indicate censoring of data. 95% CI widths have not been adjusted for multiplicity and cannot be used to infer definitive treatment effects.

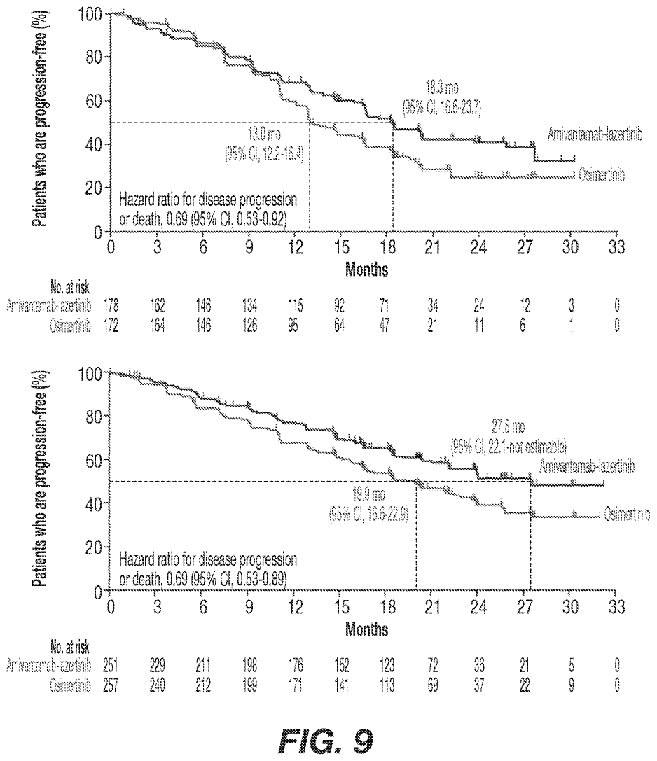

FIG. 10 shows Kaplan-Meier estimates of extracranial progression-free survival as assessed by blinded independent central review in the efficacy population. The efficacy population included all patients who had undergone randomization. Extracranial progression-free survival was defined as time from randomization to disease progression (detected by extracranial scans) or death. If first progression was detected solely in the CNS, these patients were censored at the time of CNS disease progression. The dashed lines indicate the median extracranial progression-free survival in the two groups; tick marks indicate censoring of data. 95% CI widths have not been adjusted for multiplicity and cannot be used to infer definitive treatment effects. CNS denotes central nervous system. Since MARIPOSA performed serial brain imaging, a sensitivity analysis was conducted censoring CNS-only, first progression events. The median extracranial progression-free survival was 27.5 months (95% CI, 22.1 to not estimable) in the amivantamab-lazertinib group and 18.4 months (95% CI, 16.5 to 20.2) in the osimertinib group.

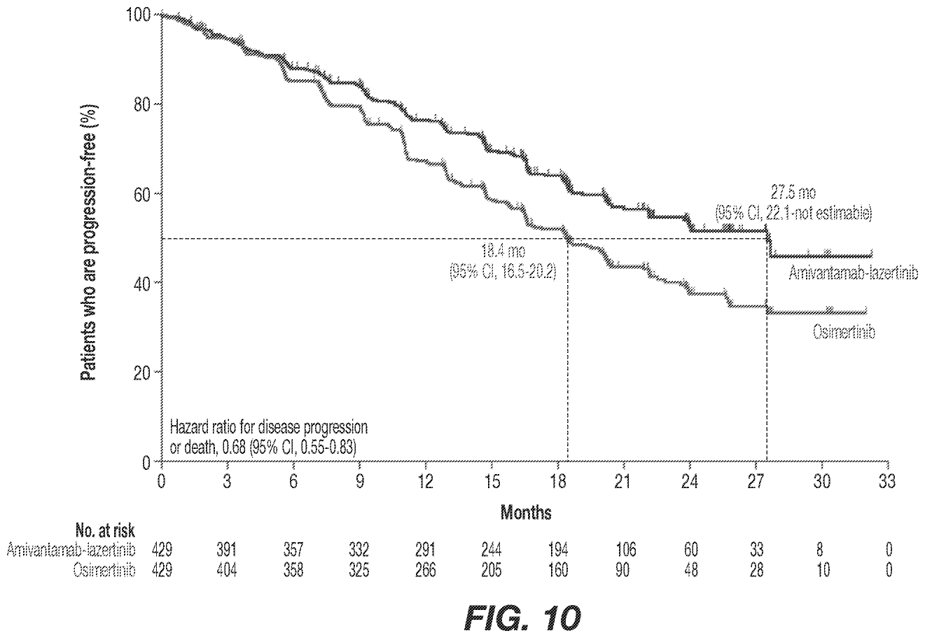

FIG. 11 shows waterfall plots of the best percent change from baseline in target lesions in the amivantamab-lazertinib group (top) and in the osimertinib group (bottom). The number of patients with measurable disease at baseline was 421 in amivantamab-lazertinib group and 414 in the osimertinib group, as determined by blinded independent central review. Target lesions were measured as the sum of the diameters. The objective response rate was 86% (95% CI, 83 to 89) in the amivantamab-lazertinib group and 85% (95% CI, 81 to 88) in the osimertinib group.

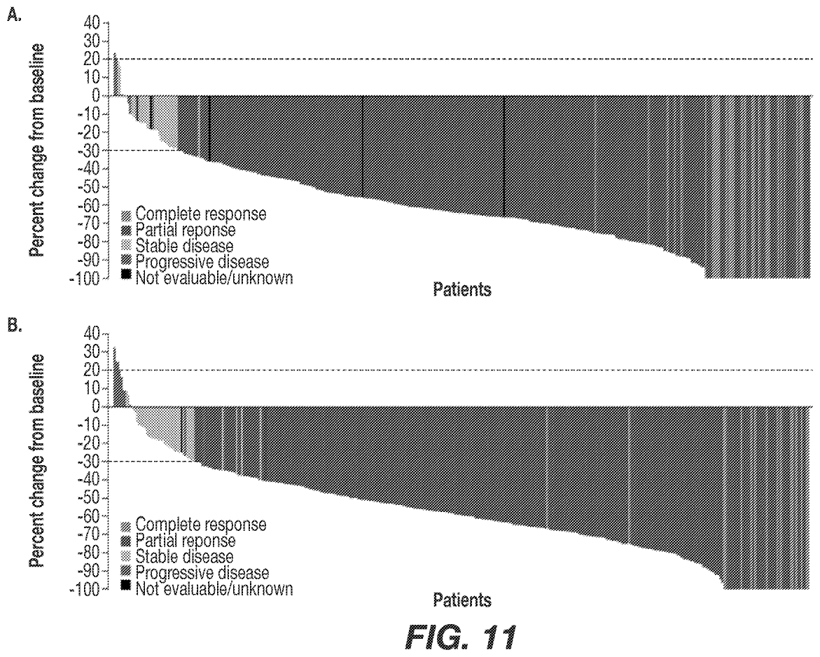

FIG. 12 shows a Kaplan-Meier estimate of response duration among confirmed responders in the efficacy population. The efficacy population included all patients who had undergone randomization. Included in this analysis were the 336 confirmed responders (out of the 421 patients with measurable disease at baseline by blinded independent central review) in the amivantamab-lazertinib group and the 314 confirmed responders (out of 414 patients) in the osimertinib group. Tick marks indicate censoring of data. The objective response rate was 86% (95% CI, 83 to 89) in the amivantamab-lazertinib group and 85% (95% CI, 81 to 88) in the osimertinib group, with median response duration among confirmed responders of 25.8 months (95% CI, 20.1 to not estimable) and 16.8 months (95% CI, 14.8 to 18.5), respectively.

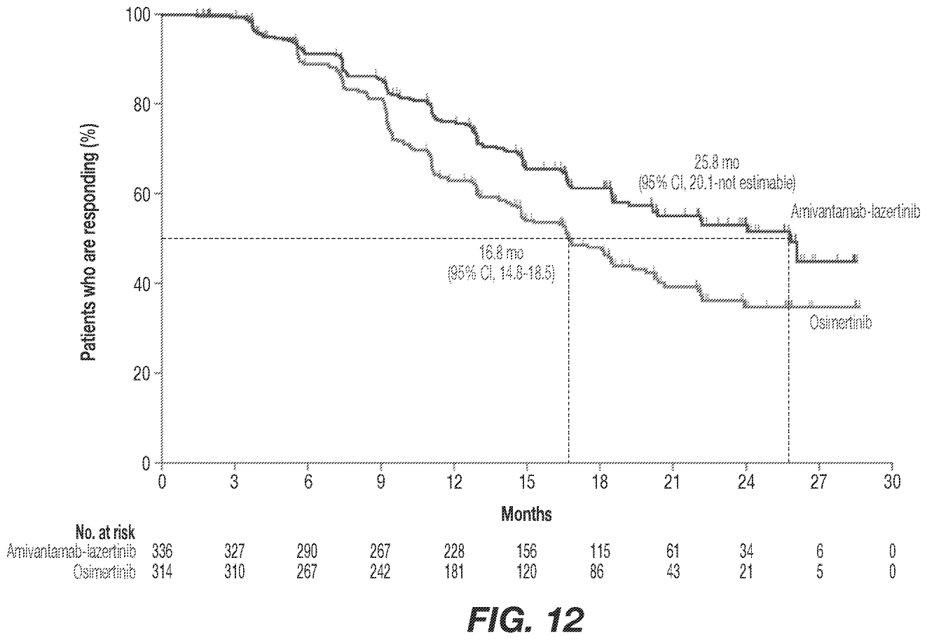

FIG. 13 shows a Kaplan-Meier estimate of time to treatment discontinuation in the efficacy population. The efficacy population included all patients who had undergone randomization. Tick marks indicate censoring of data.

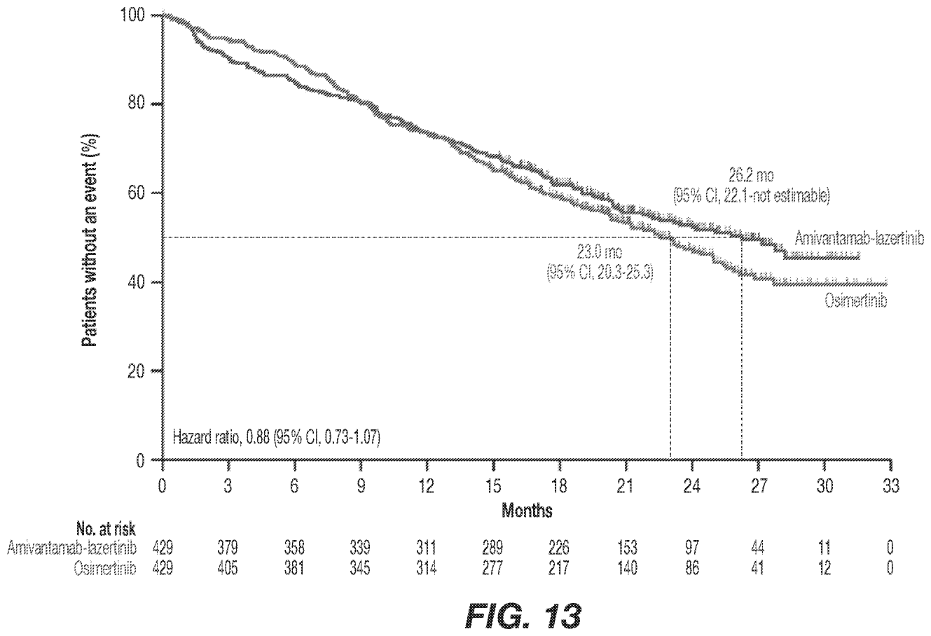

FIG. 14 shows Kaplan-Meier estimates of time to subsequent therapy in the efficacy population. The efficacy population included all patients who had undergone randomization. Tick marks indicate censoring of data.

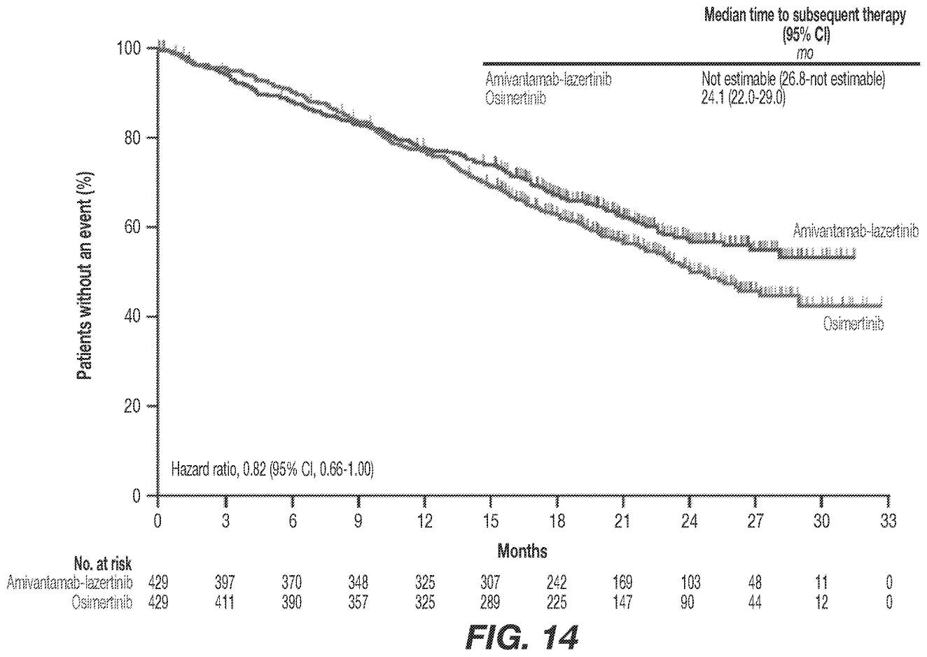

FIG. 15 shows a Kaplan-Meier estimate of progression-free survival after first subsequent therapy, which was defined from the time from randomization until the date of objective disease progression (by investigator) or death after initiation of subsequent anticancer systemic therapy, whichever occurs first. The efficacy population included all patients who had undergone randomization. Tick marks indicate censoring of data.

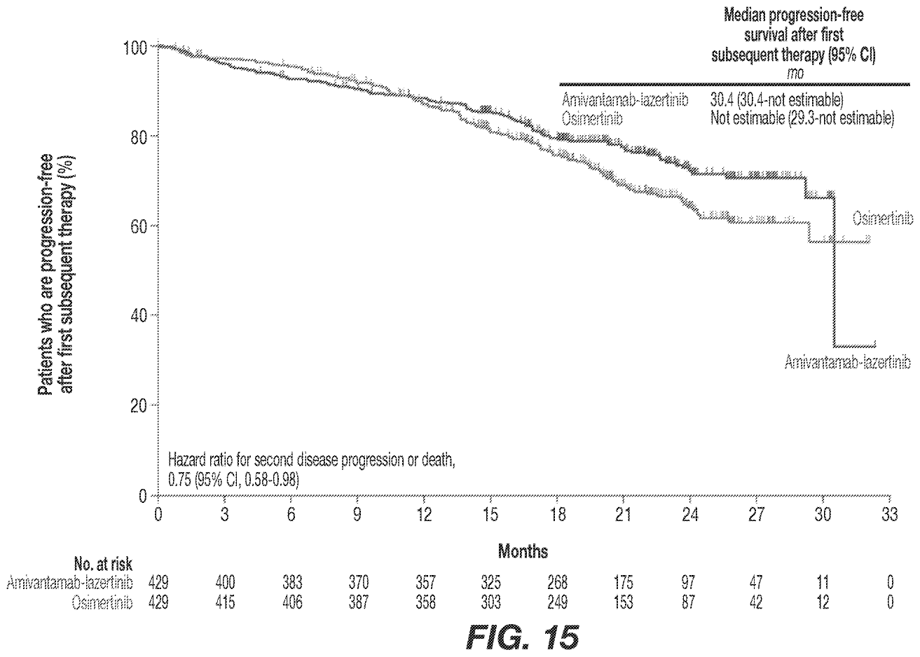

FIG. 16 shows MARIPOSA study design and methods for high risk subgroups. Detection of circulating tumor (ctDNA) and co-mutations were analyzed by next-generation sequencing (NGS) of blood at baseline. Detection and clearance of Ex19del and L858R ctDNA in blood were analyzed with droplet digital PCR (ddPCR) at baseline and C3D1. ^{a}Dosing (in 28-day cycles): Amivantamab: 1050 mg (1400 mg if ≥80 kg) weekly for the first 4 weeks, then every 2 weeks; Lazertinib: 240 mg daily; Osimertinib: 80 mg daily. ^{b}Lazertinib monotherapy arm was included to assess the contribution of components. ^{c}Efficacy assessments were analyzed per RECIST v1.1 every 8 weeks (±1 week) for the first 30 months and then every 12 weeks (±1 week). ^{d}Ex19del or L858R by Biodesix ddPCR.

FIG. 17 shows progression-free survival by BICR. Amivantamab+Lazertinib reduced the risk of progression or death by 30% and improved median PFS by 7.1 months.

FIG. 18 shows progression-free survival in patients with brain metastases. Osimertinib showed a median PFS of 13.0 months among patients with brain metastases at baseline, indicating a poor prognostic subgroup. Among patients with brain metastases at baseline, amivantamab+lazertinib reduced the risk of progression or death by 31% vs Osimertinib. Among patients without brain metastases at baseline, amivantamab+lazertinib showed a consistent benefit over osimertinib: Median PFS: 27.5 vs 19.9 months and HR 0.69 (95% CI, 0.53-0.89); P=0.005.

FIG. 19 shows progression-free survival in patients with liver metastases. Osimertinib showed a median PFS of 11.0 months among patients with liver metastases at baseline, indicating a poor prognostic subgroup. Among patients with liver metastases at baseline, amivantamab+lazertinib reduced the risk of progression or death by 42% vs Osimertinib. Among patients without liver metastases at baseline, amivantamab+lazertinib showed a consistent benefit over osimertinib: Median PFS: 24.0 vs 18.3 months and HR 0.74 (95% CI, 0.60-0.91); P=0.004.

FIG. 20 shows next-generation sequencing circulating tumor DNA (ctDNA) pathogenic mutation patterns at baseline. 85% (540/636 samples) had pathogenic alterations detected in ctDNA at baseline by NGS. TP53 co-mutations were observed in 56% from the amivantamab+lazertinib arm and 53% from the osimertinib arm. MET amplification occurred in 1 patient in each arm (neither with high-level amplification). Only pathogenic mutations occurring in ≥2% of patients are shown. Pathogenic mutations were detected with the Guardant Health G360® panel.

FIG. 21 shows progression-free survival in patients with TP53 co-mutations. Osimertinib showed a median PFS of 12.9 months among patients with TP53 co-mutations at baseline, indicating a poor prognostic subgroup. Among patients with TP53 co-mutations at baseline, amivantamab+lazertinib reduced the risk of progression or death by 35% vs Osimertinib. Among patients with wild-type TP53 at baseline, amivantamab+lazertinib showed a consistent benefit over osimertinib: Median PFS: 22.1 vs 19.9 months and HR 0.75 (95% CI, 0.52-1.07); P=0.114.

FIG. 22 shows detectable EGFR mutant (EGFRm) ctDNA at baseline and on treatment. Detection and clearance of Ex19del and L858R ctDNA in the blood were analyzed by ddPCR. At baseline, 336 patients in both the amivantamab+lazertinib and osimertinib arms provided analyzable ctDNA samples. Approximately 70% of patients in both arms had detectable EGFRm ctDNA at baseline. 192 patients in the amivantamab+lazertinib arm and 212 in the osimertinib arm had matched samples at baseline and C3D1 (Week 9). At C3D1 (Week 9), detectable EGFRm ctDNA was observed in 15% of these patients in both arms.

FIG. 23 shows progression-free survival in Patients With Detectable Baseline ctDNA (Ex19del or L858R by Biodesix ddPCR). Osimertinib showed a median PFS of 14.8 months among patients with detectable ctDNA at baseline, indicating a poor prognostic subgroup. Among patients with detectable baseline ctDNAa, amivantamab+lazertinib reduced the risk of progression or death by 32% vs Osimertinib. Among patients without detectable baseline ctDNA, amivantamab+lazertinib showed a consistent benefit over osimertinib: Median PFS: 27.7 vs 21.9 months and hazard ratio 0.72 (95% CI, 0.47-1.10); P=0.132. Among patients with detectable baseline ctDNA by Guardant360® NGS, amivantamab+lazertinib showed a consistent benefit over osimertinib (HR, 0.71 [95% CI, 0.57-0.89]; P=0.003).

FIG. 24 shows progression-free survival in patients without cleared ctDNA at C3D1 (Ex19del or L858R by Biodesix ddPCR. Cycles were 28 days). Osimertinib showed a median PFS of 9.1 months among patients without cleared ctDNA at C3D1, indicating a poor prognostic subgroup. Among patients without cleared ctDNA at C3D1, amivantamab+lazertinib reduced the risk of progression or death by 51% vs Osimertinib. Among patients with cleared ctDNA at C3D1, amivantamab+lazertinib showed a consistent benefit over osimertinib: Median PFS: 24.0 vs 16.5 months and HR 0.64 (95% CI, 0.48-0.87); P=0.004.

FIG. 25 shows progression-free survival for patients with high-risk features. In the MARIPOSA study, 89% of patients had at least 1 high-risk feature detected at baseline (Patients with analyzable ctDNA by NGS at baseline were included in this pooled analysis. High-risk features included baseline detectable ctDNA by NGS or baseline metastases of the liver or brain. For patients with detectable ctDNA, it was assumed TP53 co-mutations would be identified if present).

### DETAILED DESCRIPTION

### Definitions

All publications, including but not limited to patents and patent applications, cited in this specification are herein incorporated by reference as though fully set forth.

It is to be understood that the terminology used herein is for describing particular embodiments only and is not intended to be limiting. Unless defined otherwise, all technical and scientific terms used herein have the same meaning as commonly understood by one of ordinary skill in the art to which the invention pertains.

Although any methods and materials similar or equivalent to those described herein may be used in the practice for testing of the present invention, exemplary materials and methods are described herein. In describing and claiming the present invention, the following terminology will be used.

When a list is presented, unless stated otherwise, it is to be understood that each individual element of that list, and every combination of that list, is a separate embodiment. For example, a list of embodiments presented as “A, B, or C” is to be interpreted as including the embodiments, “A,” “B,” “C,” “A or B,” “A or C,” “B or C,” or “A, B, or C.”

As used in this specification and the appended claims, the singular forms “a,” “an,” and “the” include plural referents unless the content clearly dictates otherwise. Thus, for example, reference to “a cell” includes a combination of two or more cells, and the like.

The conjunctive term “and/or” between multiple recited elements is understood as encompassing both individual and combined options. For instance, where two elements are conjoined by “and/or,” a first option refers to the applicability of the first element without the second. A second option refers to the applicability of the second element without the first. A third option refers to the applicability of the first and second elements together. Any one of these options is understood to fall within the meaning, and therefore satisfy the requirement of the term “and/or” as used herein. Concurrent applicability of more than one of the options is also understood to fall within the meaning, and therefore satisfy the requirement of the term “and/or.”

The transitional terms “comprising,” “consisting essentially of,” and “consisting of” are intended to connote their generally accepted meanings in the patent vernacular; that is, (i) “comprising,” which is synonymous with “including,” “containing,” or “characterized by,” is inclusive or open-ended and does not exclude additional, unrecited elements or method steps; (ii) “consisting of” excludes any element, step, or ingredient not specified in the claim; and (iii) “consisting essentially of” limits the scope of a claim to the specified materials or steps “and those that do not materially affect the basic and novel characteristic(s)” of the claimed invention. Embodiments described in terms of the phrase “comprising” (or its equivalents) also provide as embodiments those independently described in terms of “consisting of” and “consisting essentially of.”

“Co-administration,” “administration with,” “administration in combination with,” “in combination with” or the like, encompass administration of the selected therapeutics or drugs to a single patient, and are intended to include treatment regimens in which therapeutics or drugs are administered by the same or different route of administration or at the same or different time.

“Isolated” refers to a homogenous population of molecules (such as synthetic polynucleotides, polypeptides vectors or viruses) which have been substantially separated and/or purified away from other components of the system the molecules are produced in, such as a recombinant cell, as well as a protein that has been subjected to at least one purification or isolation step. “Isolated” refers to a molecule that is substantially free of other cellular material and/or chemicals and encompasses molecules that are isolated to a higher purity, such as to 80%, 81%, 82%, 83%, 84%, 85%, 86%, 87%, 88%, 89%, 90%, 91%, 92%, 93%, 94%, 95%, 96%, 97%, 98%, 99% or 100% purity.

“Treat”, “treating” or “treatment” of a disease or disorder such as cancer refers to accomplishing one or more of the following: reducing the severity and/or duration of the disorder, inhibiting worsening of symptoms characteristic of the disorder being treated, limiting or preventing recurrence of the disorder in subjects that have previously had the disorder, or limiting or preventing recurrence of symptoms in subjects that were previously symptomatic for the disorder.

“Prevent”, “preventing”, “prevention”, or “prophylaxis” of a disease or disorder means preventing that a disorder occurs in subject.

“Diagnosing” or “diagnosis” refers to methods to determine if a subject is suffering from a given disease or condition or may develop a given disease or condition in the future or is likely to respond to treatment for a prior diagnosed disease or condition, i.e., stratifying a patient population on likelihood to respond to treatment. Diagnosis is typically performed by a physician based on the general guidelines for the disease to be diagnosed or other criteria that indicate a subject is likely to respond to a particular treatment.

“Responsive”, “responsiveness” or “likely to respond” refers to any kind of improvement or positive response, such as alleviation or amelioration of one or more symptoms, diminishment of extent of disease, stabilized (i.e., not worsening) state of disease, preventing spread of disease, delay or slowing of disease progression, amelioration or palliation of the disease state, and remission (whether partial or total), whether detectable or undetectable.

“Newly diagnosed” refers to a subject who has been diagnosed with cancer (e.g., EGFR or c-Met expressing cancer) but has not yet received treatment (e.g., treatment for lung cancer).

“Therapeutically effective amount” refers to an amount effective, at doses and for periods of time necessary, to achieve a desired therapeutic result. A therapeutically effective amount may vary depending on factors such as the disease state, age, sex, and weight of the individual, and the ability of a therapeutic or a combination of therapeutics to elicit a desired response in the individual. Exemplary indicators of an effective therapeutic or combination of therapeutics that include, for example, improved well-being of the patient.

“Refractory” refers to a disease that does not respond to a treatment. A refractory disease can be resistant to a treatment before or at the beginning of the treatment, or a refractory disease can become resistant during a treatment.

“Relapsed” refers to the return of a disease or the signs and symptoms of a disease after a period of improvement after prior treatment with a therapeutic.

“Subject” includes any human or nonhuman animal. “Nonhuman animal” includes all vertebrates, e.g., mammals and non-mammals, such as nonhuman primates, sheep, dogs, cats, horses, cows, chickens, amphibians, reptiles, etc. The terms “subject” and “patient” are used interchangeably herein.

“Reference subject” or “reference population of subjects” refers to a subject or population of subjects having a locally advanced or metastatic EGFR-mutated NCSLC harboring one or more epidermal growth factor receptor (EGFR) mutations who are treatment-naïve and who have been administered osimertinib or lazertinib without the bispecific anti-EGFR/c-Met antibody instead of a combination therapy comprising the disclosed bispecific anti-EGFR/c-Met antibody and lazertinib. The reference subject or the reference population of subjects can have substantially the same disease progression as the subject or population of subjects that are treated with the combination therapy comprising the disclosed bispecific anti-EGFR/c-Met antibody and lazertinib. In some embodiments, the population of subjects and reference population of subjects contain at least two subjects. In some embodiments, the population of subjects and reference population of subjects contain a number of subjects that allow for a statistically significant analysis of the improvement in safety and efficacy.

“About” means within an acceptable error range for the particular value as determined by one of ordinary skill in the art, which will depend in part on how the value is measured or determined, i.e., the limitations of the measurement system. Unless explicitly stated otherwise within the Examples or elsewhere in the Specification in the context of a particular assay, result or embodiment, “about” means within one standard deviation per the practice in the art, or a range of up to 5%, whichever is larger.

“Cancer” refers to an abnormal growth of cells which tend to proliferate in an uncontrolled way and, in some cases, to metastasize (spread) to other areas of a patient's body.

“EGFR or c-Met expressing cancer” refers to cancer that has detectable expression of EGFR or c-Met or has EGFR or c-Met mutation or amplification. EGFR or c-Met expression, amplification and mutation status can be detected using know methods, such as sequencing, fluorescent in situ hybridization, immunohistochemistry, flow cytometry or western blotting.

“Epidermal growth factor receptor” or “EGFR” refers to the human EGFR (also known as HER1 or ErbB1 (Ullrich et al., Nature 309:418-425, 1984)) having the amino acid sequence shown in GenBank accession number NP_005219, as well as naturally-occurring variants thereof.

“Hepatocyte growth factor receptor” or “c-Met” as used herein refers to the human c-Met having the amino acid sequence shown in GenBank Accession No: NP_001120972 and natural variants thereof.

“Bispecific anti-EGFR/c-Met antibody” or “bispecific EGFR/c-Met antibody” refers to a bispecific antibody having a first domain that specifically binds EGFR and a second domain that specifically binds c-Met. The domains specifically binding EGFR and c-Met are typically VH/VL pairs, and the bispecific anti-EGFR/c-Met antibody is monovalent in terms of binding to EGFR and c-Met.

“Specific binding” or “specifically binds” or “specifically binding” or “binds” refer to an antibody binding to an antigen or an epitope within the antigen with greater affinity than for other antigens. Typically, the antibody binds to the antigen or the epitope within the antigen with an equilibrium dissociation constant (K_{D}) of about 5×10⁻⁸ M or less, for example about 1×10⁻⁹ M or less, about 1×10⁻¹⁰ M or less, about 1×10⁻¹¹ M or less, or about 1×10⁻¹² M or less, typically with the K_{D} that is at least one hundred-fold less than its K_{D} for binding to a non-specific antigen (e.g., BSA, casein). The dissociation constant may be measured using known protocols. Antibodies that bind to the antigen or the epitope within the antigen may, however, have cross-reactivity to other related antigens, for example to the same antigen from other species (homologs), such as human or monkey, for example Macaca fascicularis (cynomolgus, cyno) or Pan troglodytes (chimpanzee, chimp). While a monospecific antibody binds one antigen or one epitope, a bispecific antibody binds two distinct antigens or two distinct epitopes.

“Antibodies” is meant in a broad sense and includes immunoglobulin molecules including monoclonal antibodies including murine, human, humanized and chimeric monoclonal antibodies, antigen binding fragments, multispecific antibodies, such as bispecific, trispecific, tetraspecific etc., dimeric, tetrameric or multimeric antibodies, single chain antibodies, domain antibodies and any other modified configuration of the immunoglobulin molecule that comprises an antigen binding site of the required specificity. “Full length antibodies” are comprised of two heavy chains (HC) and two light chains (LC) inter-connected by disulfide bonds as well as multimers thereof (e.g., IgM). Each heavy chain is comprised of a heavy chain variable region (VH) and a heavy chain constant region (comprised of domains CH1, hinge, CH2 and CH3). Each light chain is comprised of a light chain variable region (VL) and a light chain constant region (CL). The VH and the VL regions may be further subdivided into regions of hypervariability, termed complementarity determining regions (CDR), interspersed with framework regions (FR). Each VH and VL is composed of three CDRs and four FR segments, arranged from amino-to-carboxy-terminus in the following order: FR1, CDR1, FR2, CDR2, FR3, CDR3 and FR4.

“Complementarity determining regions” (CDR) are antibody regions that bind an antigen. CDRs may be defined using various delineations such as Kabat (Wu et al. (1970) J Exp Med 132:211-50) (Kabat et al., Sequences of Proteins of Immunological Interest, 5th Ed. Public Health Service, National Institutes of Health, Bethesda, Md., 1991), Chothia (Chothia et al. (1987) J Mol Biol 196:901-17), IMGT (Lefranc et al. (2003) Dev Comp Immunol 27:55-77) and AbM (Martin and Thornton (1996) J Bmol Biol 263:800-15). The correspondence between the various delineations and variable region numbering are described (see e.g., Lefranc et al. (2003) Dev Comp Immunol 27:55-77; Honegger and Pluckthun, (2001) J Mol Biol 309:657-70; International ImMunoGeneTics (IMGT) database; Web resources, imgt_org). Available programs such as abYsis by UCL Business PLC may be used to delineate CDRs. The term “CDR”, “HCDR1”, “HCDR2”, “HCDR3”, “LCDR1”, “LCDR2” and “LCDR3” as used herein includes CDRs defined by any of the methods described supra, Kabat, Chothia, IMGT or AbM, unless otherwise explicitly stated in the specification.

Immunoglobulins may be assigned to five major classes, IgA, IgD, IgE, IgG and IgM, depending on the heavy chain constant domain amino acid sequence. IgA and IgG are further sub-classified as the isotypes IgA1, IgA2, IgG1, IgG2, IgG3 and IgG4. Antibody light chains of any vertebrate species may be assigned to one of two clearly distinct types, namely kappa (κ) and lambda (λ), based on the amino acid sequences of their constant domains.

“Antigen binding fragment” refers to a portion of an immunoglobulin molecule that binds an antigen. Antigen binding fragments may be synthetic, enzymatically obtainable or genetically engineered polypeptides and include the VH, the VL, the VH and the VL, Fab, F(ab′)2, Fd and Fv fragments, domain antibodies (dAb) consisting of one VH domain or one VL domain, shark variable IgNAR domains, camelized VH domains, minimal recognition units consisting of the amino acid residues that mimic the CDRs of an antibody, such as FR3-CDR3-FR4 portions, the HCDR1, the HCDR2 and/or the HCDR3 and the LCDR1, the LCDR2 and/or the LCDR3. VH and VL domains may be linked together via a synthetic linker to form various types of single chain antibody designs where the VH/VL domains may pair intramolecularly, or intermolecularly in those cases when the VH and VL domains are expressed by separate single chain antibody constructs, to form a monovalent antigen binding site, such as single chain Fv (scFv) or diabody; described for example in Int. Patent Publ. Nos. WO1998/44001, WO1988/01649, WO1994/13804 and WO1992/01047.

“Monoclonal antibody” refers to an antibody obtained from a substantially homogenous population of antibody molecules, i.e., the individual antibodies comprising the population are identical except for possible well-known alterations such as removal of C-terminal lysine from the antibody heavy chain or post-translational modifications such as amino acid isomerization or deamidation, methionine oxidation or asparagine or glutamine deamidation. Monoclonal antibodies typically bind one antigenic epitope. A bispecific monoclonal antibody binds two distinct antigenic epitopes. Monoclonal antibodies may have heterogeneous glycosylation within the antibody population. Monoclonal antibody may be monospecific or multispecific such as bispecific, monovalent, bivalent or multivalent.

“Recombinant” refers to DNA, antibodies and other proteins that are prepared, expressed, created or isolated by recombinant means when segments from different sources are joined to produce recombinant DNA, antibodies or proteins.

“Bispecific” refers to an antibody that specifically binds two distinct antigens or two distinct epitopes within the same antigen. The bispecific antibody may have cross-reactivity to other related antigens, for example to the same antigen from other species (homologs), such as human or monkey, for example Macaca cynomolgus (cynomolgus, cyno) or Pan troglodytes, or may bind an epitope that is shared between two or more distinct antigens.

“Biosimilar” (of an approved reference product/biological drug, i.e., reference listed drug) refers to a biological drug that is highly similar to the reference drug with no clinically meaningful differences between the biosimilar and the reference drug in terms of safety, purity and potency, based upon data derived from (a) analytical studies that demonstrate that the biological product is highly similar to the reference drug; (b) animal studies (including the assessment of toxicity); and/or (c) a clinical study or studies (including the assessment of immunogenicity and pharmacokinetics or pharmacodynamics) that are sufficient to demonstrate safety, purity, and potency in one or more appropriate conditions of use for which the reference product is licensed and intended to be used and for which licensure is sought for the biosimilar. The biosimilar may be an interchangeable product that may be substituted for the reference product at the pharmacy without the intervention of the prescribing healthcare professional. To meet the additional standard of “interchangeability,” the biosimilar is to be expected to produce the same clinical result as the reference product in any given patient and, if the biosimilar is administered more than once to an individual, the risk in terms of safety or diminished efficacy of alternating or switching between the use of the biosimilar and the reference product is not greater than the risk of using the reference product without such alternation or switch. The biosimilar utilizes the same mechanisms of action for the proposed conditions of use to the extend the mechanisms are known for the reference product. The condition or conditions of use prescribed, recommended, or suggested in the labeling proposed for the biosimilar have been previously approved for the reference product. The route of administration, the dosage form, and/or the strength of the biosimilar are the same as those of the reference product and the biosimilar is manufactured, processed, packed or held in a facility that meets standards designed to assure that the biosimilar continues to be safe, pure and potent. The biosimilar may include minor modifications in the amino acid sequence when compared to the reference product, such as N- or C-terminal truncations that are not expected to change the biosimilar performance.

“Antagonist” or “inhibitor” refers to a molecule that, when bound to a cellular protein, suppresses at least one reaction or activity that is induced by a natural ligand of the protein. A molecule is an antagonist when the at least one reaction or activity is suppressed by at least about 20%, 30%, 40%, 45%, 50%, 55%, 60%, 65%, 70%, 75%, 80%, 85%, 90%, 95%, or 100% more than the at least one reaction or activity suppressed in the absence of the antagonist (e.g., negative control), or when the suppression is statistically significant when compared to the suppression in the absence of the antagonist.

“PD-(L)1 axis inhibitor” refers to a molecule that inhibits PD-1 downstream signaling. PD-(L)1 axis inhibitor may be a molecule that binds PD-1, PD-L1 or PD-L2.

“Biological sample” refers to a collection of similar fluids, cells, or tissues isolated from a subject, as well as fluids, cells, or tissues present within a subject. Exemplary samples are biological fluids such as blood, serum and serosal fluids, plasma, lymph, urine, saliva, cystic fluid, tear drops, feces, sputum, mucosal secretions of the secretory tissues and organs, vaginal secretions, ascites fluids, fluids of the pleural, pericardial, peritoneal, abdominal and other body cavities, fluids collected by bronchial lavage, synovial fluid, liquid solutions contacted with a subject or biological source, for example, cell and organ culture medium including cell or organ conditioned medium, lavage fluids and the like, tissue biopsies, tumor tissue biopsies, tumor tissue samples, fine needle aspirations, surgically resected tissue, organ cultures or cell cultures. As a non-limiting example, the biological sample is a blood sample. As another non-limiting example, the biological sample is a plasma sample. As yet another non-limiting example, the biological sample is a tumor sample. In some embodiments, the biological sample is circulating tumor DNA (ctDNA) that may be isolated from various other biological samples disclosed herein such as, but not limited to, a blood or plasma sample. In some embodiments, the biological sample is tumor DNA that may be isolated from, e.g., a tumor sample.

“Low fucose” or “low fucose content” as used in the application refers to antibodies with fucose content of about between 1%-15%.

“Normal fucose” or “normal fucose content” as used herein refers to antibodies with fucose content of about over 50%, typically about over 80% or over 85%.

As used herein, “treatment naïve” refers to a subject that has been diagnosed with locally advanced or metastatic NSCLC and has not yet received anti-cancer treatment for the NSCLC; the subject is therefore chemotherapy naïve and TKI naive, e.g., has not received chemotherapy, or a tyrosine kinase inhibitor (including 1st generation TKI, 2nd generation TKI or 3rd generation TKI), or other anti-NSCLC treatment. A method of treating a treatment naïve subject may also be referred to as first-line or front line treatment.

As used herein, RECIST v1.1 criteria refer to publicly available guidelines for response evaluation criteria in solid tumors as described by Eisenhauer E A, Therasse P, Bogaerts J, et al. New response evaluation criteria in solid tumours: revised RECIST guideline (version 1.1). Eur J Cancer. 2009; 45 (2): 228-247, which is incorporated by reference herein. Eisenhauer et al., provide the following definitions of criteria used to determine objective tumor response for target lesions:
Complete Response (CR): Disappearance of all target lesions. Any pathological lymph nodes (whether target or non-target) must have reduction in short axis to <10 mm.
Partial Response (PR): At least a 30% decrease in the sum of diameters of target lesions, taking as reference the baseline sum diameters.
Progressive Disease (PD): At least a 20% increase in the sum of diameters of target lesions, taking as reference the smallest sum on study (this includes the baseline sum if that is the smallest on study). In addition to the relative increase of 20%, the sum must also demonstrate an absolute increase of at least 5 mm. (Note: the appearance of one or more new lesions is also considered progression).
Stable Disease (SD): Neither sufficient shrinkage to qualify for PR nor sufficient increase to qualify for PD, taking as reference the smallest sum diameters while on study.

As used herein, a partial response or better refers to a partial response (PR) or complete response (CR); and progression-free refers to lack of disease progression.

### Methods of the Disclosure

New treatment options for patients that are newly diagnosed with EGFR mutation-positive advanced NSCLC represent a significant unmet medical need. The EGFR-TKI TAGRISSO® (osimertinib) is a standard of care in the front-line patient population. Results of the FLAURA clinical trial showed that in patients with previously untreated EGFR mutation-positive advanced NSCLC, osimertinib treatment resulted in a median progression-free survival (mPFS) of 18.9 months (Soria et al., N Engl J Med 2018; 378:113-125).

The present inventors have developed novel methods for treating EGFR mutation-positive advanced NSCLC in patients that are treatment naïve. The present inventors have further developed methods of improving median progression free survival and/or improving overall survival for patients with locally advanced or metastatic non-small cell lung cancer (NSCLC) harboring one or more epidermal growth factor receptor (EGFR) mutations who are treatment-naïve. Thus, embodiments of the present invention provide methods for treating, methods of improving median progression free survival, and methods of improving overall survival for patients and/or populations of patients with newly diagnosed EGFR-mutant non-small cell lung cancer (NSCLC).

Disclosed herein are methods of treating non-small cell lung cancer (NSCLC) in a subject in need thereof comprising administering to the subject a combination therapy comprising a therapeutically effective amount of a bispecific anti-EGFR/c-Met antibody; and a therapeutically effective amount of lazertinib, or a pharmaceutically acceptable salt or hydrate thereof, wherein the subject has been diagnosed with locally advanced or metastatic NSCLC harboring one or more epidermal growth factor receptor (EGFR) mutations, and wherein the subject is treatment naïve.

Also disclosed herein are methods of improving median progression free survival (PFS) in a population of subjects with locally advanced or metastatic non-small cell lung cancer (NSCLC) harboring one or more epidermal growth factor receptor (EGFR) mutations who are treatment-naïve, the method comprising administering to the population of subjects a combination therapy comprising a therapeutically effective amount of a bispecific anti-EGFR/c-Met antibody, and a therapeutically effective amount of lazertinib, or a pharmaceutically acceptable salt or hydrate thereof, and wherein the improvement in median PFS is relative to median PFS of a reference population of subjects with NSCLC harboring one or more EGFR mutations who are treatment-naïve, the reference population having been administered osimertinib or lazertinib without the bispecific anti-EGFR/c-Met antibody.

Also disclosed herein are methods of improving median progression free survival (PFS) in a population of treatment-naïve subjects diagnosed with locally advanced or metastatic non-small cell lung cancer (NSCLC) with baseline brain metastases, wherein the NSCLC harbors one or more exon 19 deletions or an exon 21 L858R substitution, the methods comprising administering to the population of subjects a combination therapy comprising: a therapeutically effective amount of a bispecific anti-EGFR/c-Met antibody, and a therapeutically effective amount of lazertinib, or a pharmaceutically acceptable salt or hydrate thereof, and wherein the improvement in median PFS is relative to median PFS of a reference population of treatment-naive subjects with NSCLC with baseline brain metastases, wherein the NSCLC harbors one or more exon 19 deletions or an exon 21 L858R substitution, the reference population having been administered osimertinib or lazertinib without the bispecific anti-EGFR/c-Met antibody.

Also disclosed herein are methods of improving median progression free survival (PFS) in a population of treatment-naïve subjects diagnosed with locally advanced or metastatic non-small cell lung cancer (NSCLC) without baseline brain metastases, wherein the NSCLC harbors one or more exon 19 deletions or an exon 21 L858R substitution, the methods comprising administering to the population of subjects a combination therapy comprising: a therapeutically effective amount of a bispecific anti-EGFR/c-Met antibody, and a therapeutically effective amount of lazertinib, or a pharmaceutically acceptable salt or hydrate thereof, and wherein the improvement in median PFS is relative to median PFS of a reference population of treatment-naive subjects with NSCLC without baseline brain metastases, wherein the NSCLC harbors one or more exon 19 deletions or an exon 21 L858R substitution, the reference population having been administered osimertinib or lazertinib without the bispecific anti-EGFR/c-Met antibody.

Also disclosed herein are methods of improving median progression free survival (PFS) in a population of treatment-naïve subjects diagnosed with locally advanced or metastatic non-small cell lung cancer (NSCLC) with baseline liver metastases, wherein the NSCLC harbors one or more exon 19 deletions or an exon 21 L858R substitution, the methods comprising administering to the population of subjects a combination therapy comprising: a therapeutically effective amount of a bispecific anti-EGFR/c-Met antibody, and a therapeutically effective amount of lazertinib, or a pharmaceutically acceptable salt or hydrate thereof, and wherein the improvement in median PFS is relative to median PFS of a reference population of treatment-naive subjects with NSCLC with baseline liver metastases, wherein the NSCLC harbors one or more exon 19 deletions or an exon 21 L858R substitution, the reference population having been administered osimertinib or lazertinib without the bispecific anti-EGFR/c-Met antibody.

Also disclosed herein are methods of improving median progression free survival (PFS) in a population of treatment-naïve subjects diagnosed with locally advanced or metastatic non-small cell lung cancer (NSCLC) without baseline liver metastases, wherein the NSCLC harbors one or more exon 19 deletions or an exon 21 L858R substitution, the methods comprising administering to the population of subjects a combination therapy comprising: a therapeutically effective amount of a bispecific anti-EGFR/c-Met antibody, and a therapeutically effective amount of lazertinib, or a pharmaceutically acceptable salt or hydrate thereof, and wherein the improvement in median PFS is relative to median PFS of a reference population of treatment-naive subjects with NSCLC without baseline liver metastases, wherein the NSCLC harbors one or more exon 19 deletions or an exon 21 L858R substitution, the reference population having been administered osimertinib or lazertinib without the bispecific anti-EGFR/c-Met antibody.

Also disclosed herein are methods of improving median progression free survival (PFS) in a population of treatment-naïve subjects diagnosed with locally advanced or metastatic non-small cell lung cancer (NSCLC) with TP53 co-mutation, wherein the NSCLC harbors one or more exon 19 deletions or an exon 21 L858R substitution, the methods comprising administering to the population of subjects a combination therapy comprising: a therapeutically effective amount of a bispecific anti-EGFR/c-Met antibody, and a therapeutically effective amount of lazertinib, or a pharmaceutically acceptable salt or hydrate thereof, and wherein the improvement in median PFS is relative to median PFS of a reference population of treatment-naïve subjects with NSCLC with TP53 co-mutation, wherein the NSCLC harbors one or more exon 19 deletions or an exon 21 L858R substitution, the reference population having been administered osimertinib or lazertinib without the bispecific anti-EGFR/c-Met antibody.

Also disclosed herein are methods of improving median progression free survival (PFS) in a population of treatment-naïve subjects diagnosed with locally advanced or metastatic non-small cell lung cancer (NSCLC) without TP53 co-mutation, wherein the NSCLC harbors one or more exon 19 deletions or an exon 21 L858R substitution, the methods comprising administering to the population of subjects a combination therapy comprising: a therapeutically effective amount of a bispecific anti-EGFR/c-Met antibody, and a therapeutically effective amount of lazertinib, or a pharmaceutically acceptable salt or hydrate thereof, and wherein the improvement in median PFS is relative to median PFS of a reference population of treatment-naïve subjects with NSCLC without TP53 co-mutation, wherein the NSCLC harbors one or more exon 19 deletions or an exon 21 L858R substitution, the reference population having been administered osimertinib or lazertinib without the bispecific anti-EGFR/c-Met antibody.

Also disclosed herein are methods of improving median progression free survival (PFS) in a population of treatment-naïve subjects diagnosed with locally advanced or metastatic non-small cell lung cancer (NSCLC) with detectable baseline mutant EGFR circulating tumor DNA, wherein the NSCLC harbors one or more exon 19 deletions or an exon 21 L858R substitution, the methods comprising administering to the population of subjects a combination therapy comprising: a therapeutically effective amount of a bispecific anti-EGFR/c-Met antibody, and a therapeutically effective amount of lazertinib, or a pharmaceutically acceptable salt or hydrate thereof, and wherein the improvement in median PFS is relative to median PFS of a reference population of treatment-naïve subjects with NSCLC with detectable baseline mutant EGFR circulating tumor DNA, wherein the NSCLC harbors one or more exon 19 deletions or an exon 21 L858R substitution, the reference population having been administered osimertinib or lazertinib without the bispecific anti-EGFR/c-Met antibody.

Also disclosed herein are methods of improving median progression free survival (PFS) in a population of treatment-naïve subjects diagnosed with locally advanced or metastatic non-small cell lung cancer (NSCLC) without detectable baseline mutant EGFR circulating tumor DNA, wherein the NSCLC harbors one or more exon 19 deletions or an exon 21 L858R substitution, the methods comprising administering to the population of subjects a combination therapy comprising: a therapeutically effective amount of a bispecific anti-EGFR/c-Met antibody, and a therapeutically effective amount of lazertinib, or a pharmaceutically acceptable salt or hydrate thereof, and wherein the improvement in median PFS is relative to median PFS of a reference population of treatment-naïve subjects with NSCLC without detectable baseline mutant EGFR circulating tumor DNA, wherein the NSCLC harbors one or more exon 19 deletions or an exon 21 L858R substitution, the reference population having been administered osimertinib or lazertinib without the bispecific anti-EGFR/c-Met antibody.

Also disclosed herein are methods of improving median progression free survival (PFS) in a population of treatment-naïve subjects diagnosed with locally advanced or metastatic non-small cell lung cancer (NSCLC) without mutant EGFR circulating tumor DNA clearance at C3D1, wherein the NSCLC harbors one or more exon 19 deletions or an exon 21 L858R substitution, the methods comprising administering to the population of subjects a combination therapy comprising: a therapeutically effective amount of a bispecific anti-EGFR/c-Met antibody, and a therapeutically effective amount of lazertinib, or a pharmaceutically acceptable salt or hydrate thereof, and wherein the improvement in median PFS is relative to median PFS of a reference population of treatment-naive subjects with NSCLC without mutant EGFR circulating tumor DNA clearance at C3D1, wherein the NSCLC harbors one or more exon 19 deletions or an exon 21 L858R substitution, the reference population having been administered osimertinib or lazertinib without the bispecific anti-EGFR/c-Met antibody.

Also disclosed herein are methods of improving median progression free survival (PFS) in a population of treatment-naïve subjects diagnosed with locally advanced or metastatic non-small cell lung cancer (NSCLC) with mutant EGFR circulating tumor DNA clearance at C3D1, wherein the NSCLC harbors one or more exon 19 deletions or an exon 21 L858R substitution, the methods comprising administering to the population of subjects a combination therapy comprising: a therapeutically effective amount of a bispecific anti-EGFR/c-Met antibody, and a therapeutically effective amount of lazertinib, or a pharmaceutically acceptable salt or hydrate thereof, and wherein the improvement in median PFS is relative to median PFS of a reference population of treatment-naive subjects with NSCLC with mutant EGFR circulating tumor DNA clearance at C3D1, wherein the NSCLC harbors one or more exon 19 deletions or an exon 21 L858R substitution, the reference population having been administered osimertinib or lazertinib without the bispecific anti-EGFR/c-Met antibody.

Disclosed herein are methods of improving overall survival (OS) in a subject or a population of subjects with locally advanced or metastatic non-small cell lung cancer (NSCLC) harboring one or more epidermal growth factor receptor (EGFR) mutations who are treatment-naïve, the method comprising administering to the subject or the population of subjects a combination therapy comprising a therapeutically effective amount of a bispecific anti-EGFR/c-Met antibody, and a therapeutically effective amount of lazertinib, or a pharmaceutically acceptable salt or hydrate thereof, and wherein the improvement in OS is relative to OS of a reference subject or a reference population of subjects with NSCLC harboring one or more EGFR mutations who are treatment-naive, the reference subject or the reference population of subjects having been administered osimertinib or lazertinib without the bispecific anti-EGFR/c-Met antibody.

Also disclosed herein are methods of improving overall survival (OS) in a treatment-naïve subject or a population of treatment-naïve subjects with locally advanced or metastatic non-small cell lung cancer (NSCLC) with baseline brain metastases, wherein the NSCLC harbors one or more exon 19 deletions or an exon 21 L858R substitution, the methods comprising administering to the subject or the population of subjects a combination therapy comprising: a therapeutically effective amount of a bispecific anti-EGFR/c-Met antibody, and a therapeutically effective amount of lazertinib, or a pharmaceutically acceptable salt or hydrate thereof, and wherein the improvement in OS is relative to OS of a treatment-naïve reference subject or a reference population of treatment-naïve subjects with NSCLC with baseline brain metastases, wherein the NSCLC harbors one or more exon 19 deletions or an exon 21 L858R substitution, the reference subject or the reference population of subjects having been administered osimertinib or lazertinib without the bispecific anti-EGFR/c-Met antibody.

Also disclosed herein are methods of improving overall survival (OS) in a treatment-naïve subject or a population of treatment-naïve subjects with locally advanced or metastatic non-small cell lung cancer (NSCLC) without baseline brain metastases, wherein the NSCLC harbors one or more exon 19 deletions or an exon 21 L858R substitution, the methods comprising administering to the subject or the population of subjects a combination therapy comprising: a therapeutically effective amount of a bispecific anti-EGFR/c-Met antibody, and a therapeutically effective amount of lazertinib, or a pharmaceutically acceptable salt or hydrate thereof, and wherein the improvement in OS is relative to OS of a treatment-naïve reference subject or a reference population of treatment-naïve subjects with NSCLC without baseline brain metastases, wherein the NSCLC harbors one or more exon 19 deletions or an exon 21 L858R substitution, the reference subject or the reference population of subjects having been administered osimertinib or lazertinib without the bispecific anti-EGFR/c-Met antibody.

Also disclosed here are methods of improving overall survival (OS) in a treatment-naïve subject or a population of treatment-naïve subjects with locally advanced or metastatic non-small cell lung cancer (NSCLC) with baseline liver metastases, wherein the NSCLC harbors one or more exon 19 deletions or an exon 21 L858R substitution, the methods comprising administering to the subject or the population of subjects a combination therapy comprising: a therapeutically effective amount of a bispecific anti-EGFR/c-Met antibody, and a therapeutically effective amount of lazertinib, or a pharmaceutically acceptable salt or hydrate thereof, and wherein the improvement in OS is relative to OS of a treatment-naïve reference subject or a reference population of treatment-naïve subjects with NSCLC with baseline liver metastases, wherein the NSCLC harbors one or more exon 19 deletions or an exon 21 L858R substitution, the reference subject or the reference population of subjects having been administered osimertinib or lazertinib without the bispecific anti-EGFR/c-Met antibody.

Also disclosed here are methods of improving overall survival (OS) in a treatment-naïve subject or a population of treatment-naïve subjects with locally advanced or metastatic non-small cell lung cancer (NSCLC) without baseline liver metastases, wherein the NSCLC harbors one or more exon 19 deletions or an exon 21 L858R substitution, the methods comprising administering to the subject or the population of subjects a combination therapy comprising: a therapeutically effective amount of a bispecific anti-EGFR/c-Met antibody, and a therapeutically effective amount of lazertinib, or a pharmaceutically acceptable salt or hydrate thereof, and wherein the improvement in OS is relative to OS of a treatment-naïve reference subject or a reference population of treatment-naïve subjects with NSCLC without baseline liver metastases, wherein the NSCLC harbors one or more exon 19 deletions or an exon 21 L858R substitution, the reference subject or the reference population of subjects having been administered osimertinib or lazertinib without the bispecific anti-EGFR/c-Met antibody.

Also disclosed herein are methods of improving overall survival (OS) in a treatment-naïve subject or a population of treatment-naïve subjects with locally advanced or metastatic non-small cell lung cancer (NSCLC) with TP53 co-mutation, wherein the NSCLC harbors one or more exon 19 deletions or an exon 21 L858R substitution, the methods comprising administering to the subject or the population of subjects a combination therapy comprising: a therapeutically effective amount of a bispecific anti-EGFR/c-Met antibody, and a therapeutically effective amount of lazertinib, or a pharmaceutically acceptable salt or hydrate thereof, and wherein the improvement in OS is relative to OS of a treatment-naïve reference subject or a reference population of treatment-naïve subjects with NSCLC with TP53 co-mutation, wherein the NSCLC harbors one or more exon 19 deletions or an exon 21 L858R substitution, the reference subject or the reference population of subjects having been administered osimertinib or lazertinib without the bispecific anti-EGFR/c-Met antibody.

Also disclosed herein are methods of improving overall survival (OS) in a treatment-naïve subject or a population of treatment-naïve subjects with locally advanced or metastatic non-small cell lung cancer (NSCLC) without TP53 co-mutation, wherein the NSCLC harbors one or more exon 19 deletions or an exon 21 L858R substitution, the methods comprising administering to the subject or the population of subjects a combination therapy comprising: a therapeutically effective amount of a bispecific anti-EGFR/c-Met antibody, and a therapeutically effective amount of lazertinib, or a pharmaceutically acceptable salt or hydrate thereof, and wherein the improvement in OS is relative to OS of a treatment-naïve reference subject or a reference population of treatment-naïve subjects with NSCLC without TP53 co-mutation, wherein the NSCLC harbors one or more exon 19 deletions or an exon 21 L858R substitution, the reference subject or the reference population of subjects having been administered osimertinib or lazertinib without the bispecific anti-EGFR/c-Met antibody.

Also disclosed herein are methods of improving overall survival (OS) in a treatment-naïve subject or a population of treatment-naïve subjects with locally advanced or metastatic non-small cell lung cancer (NSCLC) with detectable baseline mutant EGFR circulating tumor DNA, wherein the NSCLC harbors one or more exon 19 deletions or an exon 21 L858R substitution, the methods comprising administering to the subject or the population of subjects a combination therapy comprising: a therapeutically effective amount of a bispecific anti-EGFR/c-Met antibody, and a therapeutically effective amount of lazertinib, or a pharmaceutically acceptable salt or hydrate thereof, and wherein the improvement in OS is relative to OS of a treatment-naïve reference subject or a reference population of treatment-naïve subjects with NSCLC with detectable baseline mutant EGFR circulating tumor DNA, wherein the NSCLC harbors one or more exon 19 deletions or an exon 21 L858R substitution, the reference subject or the reference population of subjects having been administered osimertinib or lazertinib without the bispecific anti-EGFR/c-Met antibody.

Also disclosed herein are methods of improving overall survival (OS) in a treatment-naïve subject or a population of treatment-naïve subjects with locally advanced or metastatic non-small cell lung cancer (NSCLC) without detectable baseline mutant EGFR circulating tumor DNA, wherein the NSCLC harbors one or more exon 19 deletions or an exon 21 L858R substitution, the methods comprising administering to the subject or the population of subjects a combination therapy comprising: a therapeutically effective amount of a bispecific anti-EGFR/c-Met antibody, and a therapeutically effective amount of lazertinib, or a pharmaceutically acceptable salt or hydrate thereof, and wherein the improvement in OS is relative to OS of a treatment-naïve reference subject or a reference population of treatment-naïve subjects with NSCLC without detectable baseline mutant EGFR circulating tumor DNA, wherein the NSCLC harbors one or more exon 19 deletions or an exon 21 L858R substitution, the reference subject or the reference population of subjects having been administered osimertinib or lazertinib without the bispecific anti-EGFR/c-Met antibody.

Also disclosed herein are methods of improving overall survival (OS) in a treatment-naïve subject or a population of treatment-naïve subjects with locally advanced or metastatic non-small cell lung cancer (NSCLC) without mutant EGFR circulating tumor DNA clearance at C3D1, wherein the NSCLC harbors one or more exon 19 deletions or an exon 21 L858R substitution, the methods comprising administering to the subject or the population of subjects a combination therapy comprising: a therapeutically effective amount of a bispecific anti-EGFR/c-Met antibody, and a therapeutically effective amount of lazertinib, or a pharmaceutically acceptable salt or hydrate thereof, and wherein the improvement in OS is relative to OS of a treatment-naïve reference subject or a reference population of treatment-naïve subjects with NSCLC without mutant EGFR circulating tumor DNA clearance at C3D1, wherein the NSCLC harbors one or more exon 19 deletions or an exon 21 L858R substitution, the reference subject or the reference population of subjects having been administered osimertinib or lazertinib without the bispecific anti-EGFR/c-Met antibody.

Also disclosed herein are methods of improving overall survival (OS) in a treatment-naïve subject or a population of treatment-naïve subjects with locally advanced or metastatic non-small cell lung cancer (NSCLC) with mutant EGFR circulating tumor DNA clearance at C3D1, wherein the NSCLC harbors one or more exon 19 deletions or an exon 21 L858R substitution, the methods comprising administering to the subject or the population of subjects a combination therapy comprising: a therapeutically effective amount of a bispecific anti-EGFR/c-Met antibody, and a therapeutically effective amount of lazertinib, or a pharmaceutically acceptable salt or hydrate thereof, and wherein the improvement in OS is relative to OS of a treatment-naïve reference subject or a reference population of treatment-naïve subjects with NSCLC with mutant EGFR circulating tumor DNA clearance at C3D1, wherein the NSCLC harbors one or more exon 19 deletions or an exon 21 L858R substitution, the reference subject or the reference population of subjects having been administered osimertinib or lazertinib without the bispecific anti-EGFR/c-Met antibody.

In some embodiments, the methods are performed on a subject and the methods provide an improvement in OS compared to a reference subject. In some embodiments, the methods are performed on a subject and the methods provide an improvement in OS compared to a reference population of subjects. In some embodiments, the methods are performed on a population of subjects and the methods provide an improvement in OS compared to a reference population of subjects.

In some embodiments of the disclosed methods, the lazertinib, or pharmaceutically acceptable salt or hydrate thereof, is lazertinib mesylate. In some embodiments of the disclosed methods, the lazertinib, or pharmaceutically acceptable salt or hydrate thereof, is lazertinib mesylate monohydrate.

In some embodiments of the disclosed methods, the one or more EGFR mutations comprise one or more exon 19 deletions, or exon 21 L858R substitution, or any combination thereof. In some embodiments of the disclosed methods, the one or more EGFR mutations comprise one or more exon 19 deletions. In some embodiments of the disclosed methods, the one or more EGFR mutations comprise exon 21 L858R substitution.

In some embodiments of the disclosed methods, the subject has newly diagnosed, locally advanced or metastatic NSCLC that is not amenable to curative therapy including surgical resection or chemoradiation. In some embodiments of the disclosed methods, the curative therapy includes surgical resection or chemoradiation.

In some embodiments of the disclosed methods, the method comprises administering the lazertinib, or pharmaceutically acceptable salt or hydrate thereof, in an amount of about 80 mg to about 320 mg orally once daily. In some embodiments of the disclosed methods, the method comprises administering the lazertinib, or pharmaceutically acceptable salt or hydrate thereof, in an amount of about 240 mg orally once daily.

In some embodiments of the disclosed methods, the method elicits a clinical response in the subject according to RECIST v1.1 criteria. In some embodiments of the disclosed methods, the method achieves a partial response or better in the subject according to RECIST v1.1 criteria. In some embodiments of the disclosed methods, the clinical response comprises a median duration of response (DOR) of at least 1 year, at least 2 years, or at least 3 years. In some embodiments of the disclosed methods, the clinical response comprises a median duration of response (DOR) of at least 25 months.

In some embodiments of the disclosed methods, the subject is progression-free after at least 20 months. In some embodiments of the disclosed methods, the subject is progression-free after at least 24 months. In some embodiments of the disclosed methods, the subject is progression-free after at least 30 months. In some embodiments of the disclosed methods, the subject is progression-free after at least 11 months. In some embodiments of the disclosed methods, the subject is progression-free after at least 23 months. In some embodiments of the disclosed methods, the method achieves a PFS rate of 85% at 12 months, 65% at 24 months, and 51% at 36 months in a population of the treatment naïve subjects diagnosed with locally advanced or metastatic NSCLC harboring one or more epidermal growth factor receptor (EGFR) mutations. In some embodiments of the disclosed methods, the method achieves a PFS rate of 87% at 6 months, 73% at 12 months, 60% at 18 months, 48% at 24 months, and 41% at 30 months in a population of the treatment naïve subjects diagnosed with locally advanced or metastatic NSCLC harboring one or more epidermal growth factor receptor (EGFR) mutations In some embodiments of the disclosed methods, the method achieves a PFS rate of 86% in a population of the treatment naïve subjects diagnosed with locally advanced or metastatic NSCLC harboring one or more epidermal growth factor receptor (EGFR) mutations.

In some embodiments of the disclosed methods, the bispecific anti-EGFR/c-Met antibody comprises a first domain that specifically binds EGFR and a second domain that specifically binds c-Met, wherein the first domain comprises a heavy chain complementarity determining region 1 (HCDR1) of SEQ ID NO: 1, a HCDR2 of SEQ ID NO: 2, a HCDR3 of SEQ ID NO: 3, a light chain complementarity determining region 1 (LCDR1) of SEQ ID NO: 4, a LCDR2 of SEQ ID NO: 5 and a LCDR3 of SEQ ID NO: 6, and wherein the second domain that binds c-Met comprises the HCDR1 of SEQ ID NO: 7, the HCDR2 of SEQ ID NO: 8, the HCDR3 of SEQ ID NO: 9, the LCDR1 of SEQ ID NO: 10, the LCDR2 of SEQ ID NO: 11 and the LCDR3 of SEQ ID NO: 12. In some embodiments of the disclosed methods, the first domain that specifically binds EGFR comprises a heavy chain variable region (VH) of SEQ ID NO: 13 and a light chain variable region (VL) of SEQ ID NO: 14, and the second domain that specifically binds c-Met comprises the VH of SEQ ID NO: 15 and the VL of SEQ ID NO: 16. In some embodiments of the disclosed methods, the bispecific anti-EGFR/c-Met antibody is an IgG1 isotype. In some embodiments of the disclosed methods, the bispecific anti-EGFR/c-Met antibody comprises a first heavy chain (HC1) of SEQ ID NO: 17, a first light chain (LC1) of SEQ ID NO: 18, a second heavy chain (HC2) of SEQ ID NO: 19 and a second light chain (LC2) of SEQ ID NO: 20. In some embodiments of the disclosed methods, the bispecific anti-EGFR/c-Met antibody comprises a biantennary glycan structure with a fucose content of about between 1% to about 15%.

In some embodiments of the disclosed methods, the bispecific anti-EGFR/c-Met antibody comprises a first domain that specifically binds EGFR and a second domain that specifically binds c-Met, wherein the first domain comprises a heavy chain complementarity determining region 1 (HCDR1) comprising SEQ ID NO: 1, a HCDR2 comprising SEQ ID NO: 2, a HCDR3 comprising SEQ ID NO: 3, a light chain complementarity determining region 1 (LCDR1) comprising SEQ ID NO: 4, a LCDR2 comprising SEQ ID NO: 5 and a LCDR3 comprising SEQ ID NO: 6, and wherein the second domain that binds c-Met comprises a HCDR1 comprising SEQ ID NO: 7, a HCDR2 comprising SEQ ID NO: 8, a HCDR3 comprising SEQ ID NO: 9, a LCDR1 comprising SEQ ID NO: 10, a LCDR2 comprising SEQ ID NO: 11, and a LCDR3 comprising SEQ ID NO: 12. In some embodiments of the disclosed methods, the first domain that specifically binds EGFR comprises a heavy chain variable region (VH) comprising SEQ ID NO: 13 and a light chain variable region (VL) comprising SEQ ID NO: 14, and the second domain that specifically binds c-Met comprises a VH comprising SEQ ID NO: 15 and a VL comprising SEQ ID NO: 16. In some embodiments of the disclosed methods, the bispecific anti-EGFR/c-Met antibody is an IgG1 isotype. In some embodiments of the disclosed methods, the bispecific anti-EGFR/c-Met antibody comprises a first heavy chain (HC1) comprising SEQ ID NO: 17, a first light chain (LC1) comprising SEQ ID NO: 18, a second heavy chain (HC2) comprising SEQ ID NO: 19 and a second light chain (LC2) comprising SEQ ID NO: 20. In some embodiments of the disclosed methods, the bispecific anti-EGFR/c-Met antibody comprises a biantennary glycan structure with a fucose content of about between 1% to about 15%.

In some embodiments of the disclosed methods, the bispecific anti-EGFR/c-Met antibody is administered intravenously to the subject. In some embodiments of the disclosed methods, the bispecific anti-EGFR/c-Met antibody is administered at a dose of between about 140 mg to about 2240 mg. In some embodiments of the disclosed methods, the bispecific anti-EGFR/c-Met antibody is administered at a dose of about 700 mg, about 750 mg, about 800 mg, about 850 mg, about 900 mg, about 950 mg, about 1000 mg, about 1050 mg, about 1100 mg, about 1150 mg, about 1200 mg, about 1250 mg, about 1300 mg, about 1350 mg, about 1400 mg, about 1575 mg, about 1600 mg, about 2100 mg, or about 2240 mg. In some embodiments of the disclosed methods, the bispecific anti-EGFR/c-Met antibody is administered intravenously at a dose of 1050 mg if the subject has a body weight of less than 80 kg. In some embodiments of the disclosed methods, the bispecific anti-EGFR/c-Met antibody is administered intravenously at a dose of 1400 mg if the subject has a body weight of greater than or equal to 80 kg.

In some embodiments of the disclosed methods, the bispecific anti-EGFR/c-Met antibody is administered subcutaneously or intradermally to the subject. In some embodiments of the disclosed methods, the bispecific anti-EGFR/c-Met antibody is administered subcutaneously or intradermally at a dose sufficient to achieve a therapeutic effect in the subject. In some embodiments of the disclosed methods, the bispecific anti-EGFR/c-Met antibody is administered subcutaneously at a dose of 1600 mg if the subject has a body weight of less than 80 kg or at a dose of 2240 mg if the subject has a body weight of greater than or equal to 80 kg. In some embodiments of the disclosed methods, the dosing can be once in two weeks. In some embodiments of the disclosed methods, the bispecific anti-EGFR/c-Met antibody is administered subcutaneously at a dose of 2400 mg if the subject has a body weight of less than 80 kg or at a dose of 3360 mg if the subject has a body weight of greater than or equal to 80 kg. In some embodiments of the disclosed methods, the dosing can be once in three weeks.

In some embodiments of the disclosed methods, the bispecific anti-EGFR/c-Met antibody is administered twice a week, once a week, once in two weeks, once in three weeks, or once in four weeks.

In some embodiments of the disclosed methods, the subject or population of subjects has a baseline brain metastasis, baseline liver metastasis, TP53 co-mutation, detectable baseline EGFRm ctDNA, or is without EGFRm ctDNA clearance at C3D1. In some embodiments, the subject or population of subjects has a baseline brain metastasis. In some embodiments, the subject or population of subjects has a baseline liver metastasis. In some embodiments, the subject or population of subjects has a TP53 co-mutation. In some embodiments, the subject or population of subjects has detectable baseline EGFRm ctDNA. In some embodiments, the subject or population of subjects is without EGFRm ctDNA clearance at C3D1.

In some embodiments of the disclosed methods, the subject or population of subjects does not have a baseline brain metastasis, baseline liver metastasis, TP53 co-mutation, detectable baseline EGFRm ctDNA, or is without EGFRm ctDNA clearance at C3D1. In some embodiments, the subject or population of subjects does not have a baseline brain metastasis. In some embodiments, the subject or population of subjects does not have a baseline liver metastasis. In some embodiments, the subject or population of subjects does not have a TP53 co-mutation. In some embodiments, the subject or population of subjects does not have detectable baseline EGFRm ctDNA. In some embodiments, the subject or population of subjects has EGFRm ctDNA clearance at C3D1.

In some embodiments of the disclosed methods, the bispecific anti-EGFR/c-Met antibody comprises a first domain that specifically binds EGFR and a second domain that specifically binds c-Met, wherein the first domain comprises a heavy chain complementarity determining region 1 (HCDR1) of SEQ ID NO: 1, a HCDR2 of SEQ ID NO: 2, a HCDR3 of SEQ ID NO: 3, a light chain complementarity determining region 1 (LCDR1) of SEQ ID NO: 4, a LCDR2 of SEQ ID NO: 5 and a LCDR3 of SEQ ID NO: 6, and wherein the second domain that binds c-Met comprises the HCDR1 of SEQ ID NO: 7, the HCDR2 of SEQ ID NO: 8, the HCDR3 of SEQ ID NO: 9, the LCDR1 of SEQ ID NO: 10, the LCDR2 of SEQ ID NO: 11 and the LCDR3 of SEQ ID NO: 12. In some embodiments of the disclosed methods, the first domain that specifically binds EGFR comprises a heavy chain variable region (VH) of SEQ ID NO: 13 and a light chain variable region (VL) of SEQ ID NO: 14, and the second domain that specifically binds c-Met comprises the VH of SEQ ID NO: 15 and the VL of SEQ ID NO: 16.

In some embodiments of the disclosed methods, the bispecific anti-EGFR/c-Met antibody is an IgG1 isotype.

In some embodiments of the disclosed methods, the bispecific anti-EGFR/c-Met antibody comprises a first heavy chain (HC1) of SEQ ID NO: 17, a first light chain (LC1) of SEQ ID NO: 18, a second heavy chain (HC2) of SEQ ID NO: 19 and a second light chain (LC2) of SEQ ID NO: 20.

In some embodiments of the disclosed methods, the bispecific anti-EGFR/c-Met antibody comprises a first domain that specifically binds EGFR and a second domain that specifically binds c-Met, wherein the first domain comprises a heavy chain complementarity determining region 1 (HCDR1) comprising SEQ ID NO: 1, a HCDR2 comprising SEQ ID NO: 2, a HCDR3 comprising SEQ ID NO: 3, a light chain complementarity determining region 1 (LCDR1) comprising SEQ ID NO: 4, a LCDR2 comprising SEQ ID NO: 5 and a LCDR3 comprising SEQ ID NO: 6, and wherein the second domain that binds c-Met comprises a HCDR1 comprising SEQ ID NO: 7, a HCDR2 comprising SEQ ID NO: 8, a HCDR3 comprising SEQ ID NO: 9, a LCDR1 comprising SEQ ID NO: 10, a LCDR2 comprising SEQ ID NO: 11, and a LCDR3 comprising SEQ ID NO: 12. In some embodiments of the disclosed methods, the first domain that specifically binds EGFR comprises a heavy chain variable region (VH) comprising SEQ ID NO: 13 and a light chain variable region (VL) comprising SEQ ID NO: 14, and the second domain that specifically binds c-Met comprises a VH comprising SEQ ID NO: 15 and a VL comprising SEQ ID NO: 16.

In some embodiments of the disclosed methods, the bispecific anti-EGFR/c-Met antibody is an IgG1 isotype.

In some embodiments of the disclosed methods, the bispecific anti-EGFR/c-Met antibody comprises a first heavy chain (HC1) comprising SEQ ID NO: 17, a first light chain (LC1) comprising SEQ ID NO: 18, a second heavy chain (HC2) comprising SEQ ID NO: 19, and a second light chain (LC2) comprising SEQ ID NO: 20.

In some embodiments of the disclosed methods, the bispecific anti-EGFR/c-Met antibody comprises a biantennary glycan structure with a fucose content of about between 1% to about 15%. In some embodiments, the bispecific anti-EGFR/c-Met antibody comprises a biantennary glycan structure with a fucose content of about between 2% to about 14%. In some embodiments, the bispecific anti-EGFR/c-Met antibody comprises a biantennary glycan structure with a fucose content of about between 3% to about 13%. In some embodiments, the bispecific anti-EGFR/c-Met antibody comprises a biantennary glycan structure with a fucose content of about between 4% to about 12%. In some embodiments, the bispecific anti-EGFR/c-Met antibody comprises a biantennary glycan structure with a fucose content of about between 5% to about 11%. In some embodiments, the bispecific anti-EGFR/c-Met antibody comprises a biantennary glycan structure with a fucose content of about 1%. In some embodiments, the bispecific anti-EGFR/c-Met antibody comprises a biantennary glycan structure with a fucose content of about 2%. In some embodiments, the bispecific anti-EGFR/c-Met antibody comprises a biantennary glycan structure with a fucose content of about 3%. In some embodiments, the bispecific anti-EGFR/c-Met antibody comprises a biantennary glycan structure with a fucose content of about 4%. In some embodiments, the bispecific anti-EGFR/c-Met antibody comprises a biantennary glycan structure with a fucose content of about 5%. In some embodiments, the bispecific anti-EGFR/c-Met antibody comprises a biantennary glycan structure with a fucose content of about 6%. In some embodiments, the bispecific anti-EGFR/c-Met antibody comprises a biantennary glycan structure with a fucose content of about 7%. In some embodiments, the bispecific anti-EGFR/c-Met antibody comprises a biantennary glycan structure with a fucose content of about 8%. In some embodiments, the bispecific anti-EGFR/c-Met antibody comprises a biantennary glycan structure with a fucose content of about 9%. In some embodiments, the bispecific anti-EGFR/c-Met antibody comprises a biantennary glycan structure with a fucose content of about 10%. In some embodiments, the bispecific anti-EGFR/c-Met antibody comprises a biantennary glycan structure with a fucose content of about 11%. In some embodiments, the bispecific anti-EGFR/c-Met antibody comprises a biantennary glycan structure with a fucose content of about 12%. In some embodiments, the bispecific anti-EGFR/c-Met antibody comprises a biantennary glycan structure with a fucose content of about 13%. In some embodiments, the bispecific anti-EGFR/c-Met antibody comprises a biantennary glycan structure with a fucose content of about 14%. In some embodiments, the bispecific anti-EGFR/c-Met antibody comprises a biantennary glycan structure with a fucose content of about 15%.

In some embodiments of the disclosed methods, the bispecific anti-EGFR/c-Met antibody disclosed herein may be administered in combination with a tyrosine kinase inhibitor (TKI) such as, but not limited to an epidermal growth factor receptor (EGFR TKI). Non-limiting examples of TKI are erlotinib, gefitinib, lapatinib, vandetanib, afatinib, osimertinib, lazertinib, poziotinib, criotinib, cabozantinib, capmatinib, axitinib, lenvatinib, nintedanib, regorafenib, pazopanib, sorafenib, or sunitinib. In some embodiments of the disclosed methods, the bispecific anti-EGFR/c-Met antibody disclosed herein may be administered in combination with lazertinib.

Lazertinib is an oral, third-generation, brain-penetrant EGFR TKI that targets both the T790M mutation and activating EGFR mutations while sparing wild type-EGFR. An analysis of the efficacy and safety of lazertinib from the Phase 3 LASER301 (NCT04248829) study demonstrated that lazertinib improved PFS compared to the first generation EGFR TKI gefitinib in all prespecified subgroups, including Asian patients, those with exon 21 L858R mutations and those with a history of brain metastases.

Lazertinib is described in WO 2016/060443 as N-(5-(4-(4-((dimethylamino)methyl)-3-phenyl-1H-pyrazol-1-yl)pyrimidin-2-ylamino)-4-methoxy-2-morpholinophenyl) acrylamide, depicted below as a compound of Formula I.

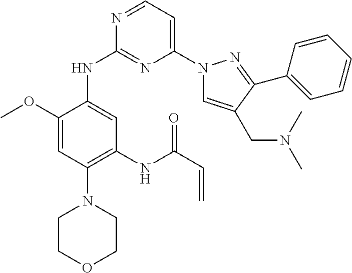 [SMILES: C=CC(=O)Nc1cc(Nc2nccc(-n3cc(CN(C)C)c(-c4ccccc4)n3)n2)c(OC)cc1N1CCOCC1]

In addition, WO2018/194356 describes salts, hydrates and crystalline forms thereof; and WO2019/022485, WO2019/022486 and WO2019/022487 disclose processes for the production of lazertinib.

Lazertinib mesylate monohydrate is depicted below as a compound of Formula Ia,

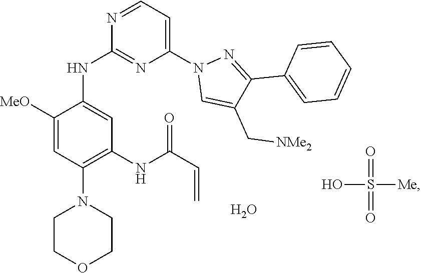

which may be referred to as N-[5-[[4-[4-[(dimethylamino)methyl]-3-phenyl-1H-pyrazol-1-yl]pyrimidin-2-yl]amino]-4-methoxy-2-(morpholin-4-yl)phenyl]acrylamide methanesulfonate hydrate.

### Therapeutic Methods

In some embodiments, the methods of the present disclosure useful for treating a cancer in a subject in need thereof may comprise administering to a subject an effective amount of a combination therapy comprising a bispecific anti-epidermal growth factor receptor (EGFR)/hepatocyte growth factor receptor (c-Met) bispecific antibody and an EGFR tyrosine kinase inhibitor (TKI).

In some embodiments, the methods of the present disclosure useful for improving median progression free survival (PFS) in a population of subjects with a cancer may comprise administering to a subject an effective amount of a combination therapy comprising a bispecific anti-epidermal growth factor receptor (EGFR)/hepatocyte growth factor receptor (c-Met) bispecific antibody and an EGFR tyrosine kinase inhibitor (TKI).

In some embodiments, the methods of the present disclosure useful for improving overall survival (OS) in a subject or a population of subjects with a cancer may comprise administering to a subject an effective amount of a combination therapy comprising a bispecific anti-epidermal growth factor receptor (EGFR)/hepatocyte growth factor receptor (c-Met) bispecific antibody and an EGFR tyrosine kinase inhibitor (TKI).

In some embodiments of the disclosed methods, the bispecific anti-EGFR/c-Met antibody comprises a first domain that specifically binds EGFR and a second domain that specifically binds c-Met, wherein the first domain comprises a heavy chain complementarity determining region 1 (HCDR1) of SEQ ID NO: 1, a HCDR2 of SEQ ID NO: 2, a HCDR3 of SEQ ID NO: 3, a light chain complementarity determining region 1 (LCDR1) of SEQ ID NO: 4, a LCDR2 of SEQ ID NO: 5 and a LCDR3 of SEQ ID NO: 6, and wherein the second domain that binds c-Met comprises the HCDR1 of SEQ ID NO: 7, the HCDR2 of SEQ ID NO: 8, the HCDR3 of SEQ ID NO: 9, the LCDR1 of SEQ ID NO: 10, the LCDR2 of SEQ ID NO: 11 and the LCDR3 of SEQ ID NO: 12. In some embodiments of the disclosed methods, the first domain that specifically binds EGFR comprises a heavy chain variable region (VH) of SEQ ID NO: 13 and a light chain variable region (VL) of SEQ ID NO: 14, and the second domain that specifically binds c-Met comprises the VH of SEQ ID NO: 15 and the VL of SEQ ID NO: 16. In some embodiments of the disclosed methods, the bispecific anti-EGFR/c-Met antibody is an IgG1 isotype. In some embodiments of the disclosed methods, the bispecific anti-EGFR/c-Met antibody comprises a first heavy chain (HC1) of SEQ ID NO: 17, a first light chain (LC1) of SEQ ID NO: 18, a second heavy chain (HC2) of SEQ ID NO: 19 and a second light chain (LC2) of SEQ ID NO: 20. In some embodiments of the disclosed methods, the bispecific anti-EGFR/c-Met antibody comprises a biantennary glycan structure with a fucose content of about between 1% to about 15%.

In some embodiments of the disclosed methods, the bispecific anti-EGFR/c-Met antibody comprises a first domain that specifically binds EGFR and a second domain that specifically binds c-Met, wherein the first domain comprises a heavy chain complementarity determining region 1 (HCDR1) comprising SEQ ID NO: 1, a HCDR2 comprising SEQ ID NO: 2, a HCDR3 comprising SEQ ID NO: 3, a light chain complementarity determining region 1 (LCDR1) comprising SEQ ID NO: 4, a LCDR2 comprising SEQ ID NO: 5, and a LCDR3 comprising SEQ ID NO: 6, and wherein the second domain that binds c-Met comprises a HCDR1 comprising SEQ ID NO: 7, a HCDR2 comprising SEQ ID NO: 8, a HCDR3 comprising SEQ ID NO: 9, a LCDR1 comprising SEQ ID NO: 10, a LCDR2 comprising SEQ ID NO: 11, and a LCDR3 comprising SEQ ID NO: 12. In some embodiments of the disclosed methods, the first domain that specifically binds EGFR comprises a heavy chain variable region (VH) comprising SEQ ID NO: 13 and a light chain variable region (VL) comprising SEQ ID NO: 14, and the second domain that specifically binds c-Met comprises a VH comprising SEQ ID NO: 15 and a VL comprising SEQ ID NO: 16. In some embodiments of the disclosed methods, the bispecific anti-EGFR/c-Met antibody is an IgG1 isotype. In some embodiments of the disclosed methods, the bispecific anti-EGFR/c-Met antibody comprises a first heavy chain (HC1) comprising SEQ ID NO: 17, a first light chain (LC1) comprising SEQ ID NO: 18, a second heavy chain (HC2) comprising SEQ ID NO: 19, and a second light chain (LC2) comprising SEQ ID NO: 20. In some embodiments of the disclosed methods, the bispecific anti-EGFR/c-Met antibody comprises a biantennary glycan structure with a fucose content of about between 1% to about 15%.

### Administration

The bispecific anti-EGFR/c-Met antibody may be administered in a pharmaceutically acceptable carrier. “Carrier” refers to a diluent, adjuvant, excipient, or vehicle with which the antibody of the invention is administered. Such vehicles may be liquids, such as water and oils, including those of petroleum, animal, vegetable or synthetic origin, such as peanut oil, soybean oil, mineral oil, sesame oil and the like. For example, 0.4% saline and 0.3% glycine may be used to formulate the bispecific anti-EGFR/c-Met antibody. These solutions are sterile and generally free of particulate matter. They may be sterilized by conventional, well-known sterilization techniques (e.g., filtration). For parenteral administration, the carrier may comprise sterile water and other excipients may be added to increase solubility or preservation. Injectable suspensions or solutions may also be prepared utilizing aqueous carriers along with appropriate additives. Suitable vehicles and formulations, inclusive of other human proteins, e.g., human serum albumin, are described, for example, in e.g., Remington: The Science and Practice of Pharmacy, 21st Edition, Troy, D. B. ed., Lipincott Williams and Wilkins, Philadelphia, PA 2006, Part 5, Pharmaceutical Manufacturing pp 691-1092, See especially pp. 958-989.

The mode of administration may be any suitable route that delivers the bispecific anti-EGFR-c-Met antibody to the host, such as parenteral administration, e.g., intradermal, intramuscular, intraperitoneal, intravenous or subcutaneous, pulmonary, transmucosal (oral, intranasal, intravaginal, rectal), using a formulation in a tablet, capsule, solution, powder, gel, particle; and contained in a syringe, an implanted device, osmotic pump, cartridge, micropump; or other means appreciated by the skilled artisan, as well known in the art. Site specific administration may be achieved by for example intratumoral, intra-articular, intrabronchial, intraabdominal, intracapsular, intracartilaginous, intracavitary, intracelial, intracerebellar, intracerebroventricular, intracolic, intracervical, intragastric, intrahepatic, intracardial, intraosteal, intrapelvic, intrapericardiac, intraperitoneal, intrapleural, intraprostatic, intrapulmonary, intrarectal, intrarenal, intraretinal, intraspinal, intrasynovial, intrathoracic, intrauterine, intravascular, intravesical, intralesional, vaginal, rectal, buccal, sublingual, intranasal, or transdermal delivery.

In some embodiments, the bispecific anti-EGFR/c-Met antibody is administered intravenously.

In some embodiments, the bispecific anti-EGFR/c-Met antibody is administered subcutaneously or intradermally to the subject. The bispecific anti-EGFR/c-Met antibody may be administered subcutaneously or intradermally at a dose sufficient to achieve a therapeutic effect in the subject.

In some embodiments, the bispecific anti-EGFR/c-Met antibody is formulated as a subcutaneous formulation as disclosed in PCT International Publication No. WO 2022/224187A1.

In some embodiments, the bispecific anti-EGFR/c-Met antibody is administered subcutaneously to the subject at a dose between about 1600 mg to about 3360 mg. The subcutaneous administration can be once in two weeks (Q2W) or once in three weeks (Q3W). Q2W dosing is administered at 1600 mg (2240 mg if the subject is ≥80 kg). Q3W dosing is administered at 2400 mg (3360 mg if the subject is ≥80 kg).

In some embodiments, the bispecific anti-EGFR/c-Met antibody is administered at a dose of between about 1400 mg to about 3360 mg. In some embodiments, the bispecific anti-EGFR/c-Met antibody is administered at a dose of between about 1400 mg to about 1750 mg.

In some embodiments, the bispecific anti-EGFR/c-Met antibody is administered at a dose of about 200 mg, about 210 mg, about 220 mg, about 230 mg, about 240 mg, about 250 mg, about 260 mg, about 270 mg, about 280 mg, about 290 mg, about 300 mg, about 310 mg, about 320 mg, about 330 mg, about 340 mg, about 350 mg, about 360 mg, about 370 mg, about 380 mg, about 390 mg, about 400 mg, about 410 mg, about 420 mg, about 430 mg, about 440 mg, about 450 mg, about 460 mg, about 470 mg, about 480 mg, about 490 mg, about 500 mg, about 510 mg, about 520 mg, about 530 mg, about 540 mg, about 550 mg, about 560 mg, about 570 mg, about 580 mg, about 590 mg, about 600 mg, about 610 mg, about 620 mg, about 630 mg, about 640 mg, about 650 mg, about 660 mg, about 670 mg, about 680 mg, about 690 mg, about 700 mg, about 710 mg, about 720 mg, about 730 mg, about 740 mg, about 750 mg, about 760 mg, about 770 mg, about 780 mg, about 790 mg, about 800 mg, about 810 mg, about 820 mg, about 830 mg, about 840 mg, about 850 mg, about 860 mg, about 870 mg, about 880 mg, about 890 mg, about 900 mg, about 910 mg, about 920 mg, about 930 mg, about 940 mg, about 950 mg, about 960 mg, about 970 mg, about 980 mg, about 990 mg, about 1000 mg, about 1010 mg, about 1020 mg, about 1030 mg, about 1040 mg, about 1050 mg, about 1060 mg, about 1070 mg, about 1080 mg, about 1090 mg, about 1100 mg, about 1110 mg, about 1120 mg, about 1130 mg, about 1140 mg, about 1150 mg, about 1160 mg, about 1170 mg, about 1180 mg, about 1190 mg, about 1200 mg, about 1210 mg, about 1220 mg, about 1230 mg, about 1240 mg, about 1250 mg, about 1260 mg, about 1270 mg, about 1280 mg, about 1290 mg, about 1300 mg, about 1310 mg, about 1320 mg, about 1330 mg, about 1340 mg, about 1350 mg, about 1360 mg, about 1370 mg, about 1380 mg, about 1390 mg, about 1400 mg, about 1410 mg, about 1420 mg, about 1430 mg, about 1440 mg, about 1450 mg, about 1460 mg, about 1470 mg, about 1480 mg, about 1490 mg, about 1500 mg, about 1510 mg, about 1520 mg, about 1530 mg, about 1540 mg, about 1550 mg, about 1560 mg, about 1570 mg, 1575 mg, about 1580 mg, about 1590 mg, about 1600 mg, about 1610 mg, 1620 mg, about 1630 mg, about 1640 mg, about 1650 mg, about 1660 mg, about 1670 mg, about 1680 mg, about 1690 mg, about 1700 mg, about 1710 mg, about 1720 mg, about 1730 mg, about 1740 mg, about 1750 mg, about 1760 mg, about 1770 mg, about 1780 mg, about 1790 mg, about 1800 mg, about 1810 mg, about 1820 mg, about 1830 mg, about 1840 mg, about 1850 mg, about 1860 mg, about 1870 mg, about 1880 mg, 1890 mg, about 1900 mg, about 1910 mg, about 1920 mg, about 1930 mg, about 1940 mg, about 1950 mg, about 1960 mg, about 1970 mg, about 1980 mg, about 1990 mg, about 2000 mg, 2100 mg, 2110 mg, 2120 mg, 2130 mg, 2140 mg, 2150 mg, 2160 mg, 2170 mg, 2180 mg, 2190 mg, 2200 mg, 2210 mg, 2220 mg, 2230 mg, 2240 mg, 2250 mg, 2260 mg, 2270 mg, 2280 mg, 2290 mg, 2300 mg, 2310 mg, 2320 mg, 2330 mg, 2340 mg, 2350 mg, 2360 mg, 2370 mg, 2380 mg, 2390 mg, 2400 mg, or 2410 mg.

In some embodiments, the bispecific anti-EGFR/c-Met antibody is administered at a dose of about 350 mg, about 700 mg, about 1050 mg, or about 1400 mg. In some embodiments, the bispecific anti-EGFR/c-Met antibody is administered at a dose of about 350 mg. In some embodiments, the bispecific anti-EGFR/c-Met antibody is administered at a dose of about 700 mg. In some embodiments, the bispecific anti-EGFR/c-Met antibody is administered at a dose of about 750 mg. In some embodiments, the bispecific anti-EGFR/c-Met antibody is administered at a dose of about 800 mg. In some embodiments, the bispecific anti-EGFR/c-Met antibody is administered at a dose of about 850 mg. In some embodiments, the bispecific anti-EGFR/c-Met antibody is administered at a dose of about 900 mg. In some embodiments, the bispecific anti-EGFR/c-Met antibody is administered at a dose of about 950 mg. In some embodiments, the bispecific anti-EGFR/c-Met antibody is administered at a dose of about 1000 mg. In some embodiments, the bispecific anti-EGFR/c-Met antibody is administered at a dose of about 1050 mg. In some embodiments, the bispecific anti-EGFR/c-Met antibody is administered at a dose of about 1100 mg. In some embodiments, the bispecific anti-EGFR/c-Met antibody is administered at a dose of about 1150 mg. In some embodiments, the bispecific anti-EGFR/c-Met antibody is administered at a dose of about 1200 mg. In some embodiments, the bispecific anti-EGFR/c-Met antibody is administered at a dose of about 1250 mg. In some embodiments, the bispecific anti-EGFR/c-Met antibody is administered at a dose of about 1300 mg. In some embodiments, the bispecific anti-EGFR/c-Met antibody is administered at a dose of about 1350 mg. In some embodiments, the bispecific anti-EGFR/c-Met antibody is administered at a dose of about 1400 mg.

In some embodiments, the bispecific anti-EGFR/c-Met antibody is administered is administered at a dose of 1050 mg if the subject has a body weight of less than 80 kg. In some embodiments, the bispecific anti-EGFR/c-Met antibody is administered at a dose of 1400 mg if the subject has a body weight of greater than or equal to 80 kg.

In some embodiments, the bispecific anti-EGFR/c-Met antibody is administered once a week. In some embodiments, the bispecific anti-EGFR/c-Met antibody is administered about 1050 mg once a week. In some embodiments, the bispecific anti-EGFR/c-Met antibody is administered about 1400 mg once a week.

In some embodiments, the bispecific anti-EGFR/c-Met antibody is administered once in two weeks. In some embodiments, the bispecific anti-EGFR/c-Met antibody is administered about 1050 mg once in two weeks. In some embodiments, the bispecific anti-EGFR/c-Met antibody is administered about 1400 mg once in two weeks.

In some embodiments, the bispecific anti-EGFR/c-Met antibody is administered twice a week. In some embodiments, the bispecific anti-EGFR/c-Met antibody is administered once a week. In some embodiments, the bispecific anti-EGFR/c-Met antibody is administered once in two weeks. In some embodiments, the bispecific anti-EGFR/c-Met antibody is administered once in three weeks. In some embodiments, the bispecific anti-EGFR/c-Met antibody is administered once in four weeks.

In some embodiments, the bispecific anti-EGFR/c-Met antibody is administered twice a week, once a week, once in two weeks, once in three weeks or once in four weeks.

In some embodiments, the mode of administration that may the suitable route that delivers lazertinib to the subject may be oral administration, such as oral administration of a tablet. Lazertinib tablet formulations suitable for oral administration in accordance with the present invention are described, for example, in WO2021/209893 and WO2020/079637, which are incorporated by reference herein.

In some embodiments, lazertinib is administered at a dose of between about 10 mg to about 400 mg. In some embodiments, lazertinib is administered at a dose of between about 20 mg to about 320 mg. In some embodiments, lazertinib is administered at a dose of between about 50 mg to about 300 mg. In some embodiments, lazertinib is administered at a dose of between about 100 mg to about 300 mg. In some embodiments, lazertinib is administered at a dose of between about 150 mg to about 280 mg. In some embodiments, lazertinib is administered at a dose of between about 200 mg to about 250 mg. In some embodiments, lazertinib is administered at a dose of between about 220 mg to about 250 mg.

In some embodiments, lazertinib is administered at a dose of about 20 mg, about 50 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 310 mg, about 320 mg, about 330 mg, about 340 mg, about 350 mg, about 360 mg, about 370 mg, about 380 mg, about 390 mg, or about 400 mg. In some embodiments, lazertinib is administered at a dose of about 240 mg.

In some embodiments, lazertinib is administered daily. In some embodiments, lazertinib is administered twice a week. In some embodiments, lazertinib is administered once a week. In some embodiments, lazertinib is administered once in two weeks. In some embodiments, lazertinib is administered once in three weeks. In some embodiments, lazertinib is administered once in four weeks.

In some embodiments, the bispecific anti-EGFR/c-Met antibody disclosed herein may be administered in combination with lazertinib, which may be administered using any of the doses and dosages disclosed herein. In some embodiments, lazertinib is administered at a dose of between about 10 mg to about 400 mg. In some embodiments, lazertinib is administered at a dose of between about 20 mg to about 320 mg. In some embodiments, lazertinib is administered at a dose of about 20 mg, about 50 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 310 mg, about 320 mg, about 330 mg, about 340 mg, about 350 mg, about 360 mg, about 370 mg, about 380 mg, about 390 mg, or about 400 mg. In some embodiments, lazertinib is administered at a dose of about 240 mg.

In some embodiments, the bispecific anti-EGFR/c-Met antibody disclosed herein may be administered in any of these doses and dosages disclosed herein in combination with lazertinib, which may be administered in any of these doses and dosages disclosed herein. As a non-limiting example, 700 mg amivantamab may be administered in combination with 240 mg lazertinib. As a non-limiting example, 1050 mg amivantamab may be administered in combination with 240 mg lazertinib. As a non-limiting example, 1050 mg amivantamab may be administered in combination with 240 mg lazertinib. As a non-limiting example, 1400 mg amivantamab may be administered in combination with 240 mg lazertinib.

In some embodiments, the bispecific anti-EGFR/c-Met antibody disclosed herein may be administered in combination with lazertinib, wherein lazertinib is administered daily, every other day, twice a week, or once a week. In some embodiments, the bispecific anti-EGFR/c-Met antibody disclosed herein may be administered in combination with lazertinib, wherein lazertinib is administered daily. In some embodiments, the bispecific anti-EGFR/c-Met antibody disclosed herein may be administered in combination with lazertinib, wherein lazertinib is administered orally.

In some embodiments, the combination therapy comprising a bispecific anti-EGFR/c-Met bispecific antibody and an EGFR TKI may further include one or more additional anti-cancer therapies.

In some embodiments, the methods of the present disclosure comprise administering to a subject a cancer therapy which does not include the combination therapy comprising a bispecific anti-EGFR/c-Met bispecific antibody and an EGFR TKI disclosed herein. In some embodiments, the cancer therapy may include any one of those described herein. As a non-limiting example, the cancer therapy that may be administered in the methods of the disclosure may comprise any number of various platinum-based chemotherapies or combinations thereof. As a non-limiting example, the platinum-based chemotherapy comprises carboplatin, cisplatin, or a combination thereof.

Additional anti-cancer therapies that may be administered in the methods of the disclosure may include any one or more of the chemotherapeutic drugs or other anti-cancer therapeutics known to those of skill in the art. Chemotherapeutic agents are chemical compounds useful in the treatment of cancer and include growth inhibitory agents or other cytotoxic agents and include alkylating agents, anti-metabolites, anti-microtubule inhibitors, topoisomerase inhibitors, receptor tyrosine kinase inhibitors, angiogenesis inhibitors and the like. Examples of chemotherapeutic agents include alkylating agents such as thiotepa and cyclosphosphamide (CYTOXAN®); alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamine; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, carminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-FU; folic acid analogues such as denopterin, methotrexate, pteropterin, trimetrexate; purine analogues such as fludarabine, 6-mercaptopurine, thiamiprine, thioguanine; pyrimidine analogues such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elfornithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK®; razoxane; sizofiran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (“Ara-C”); cyclophosphamide; thiotepa; members of taxoid or taxane family, such as paclitaxel (TAXOL® docetaxel (TAXOTERE®) and analogues thereof; chlorambucil; gemcitabine; 6-thioguanine; mercaptopurine; methotrexate; platinum analogues such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-11; topoisomerase inhibitor RFS 2000; difluoromethylornithine (DMFO); retinoic acid; esperamicins; capecitabine; inhibitors of receptor tyrosine kinases and/or angiogenesis, including sorafenib (NEXAVAR®), sunitinib (SUTENT®), pazopanib (VOTRIENT™), toceranib (PALLADIA™), vandetanib (ZACTIMA™), cediranib (RECENTIN®), regorafenib (BAY 73-4506), axitinib (AG013736), lestaurtinib (CEP-701), erlotinib (TARCEVA®), gefitinib (IRESSA®), afatinib (BIBW 2992), lapatinib (TYKERB®), neratinib (HKI-272), and the like, and pharmaceutically acceptable salts, acids or derivatives of any of the above. Also included in this definition are anti-hormonal agents that act to regulate or inhibit hormone action on tumors such as anti-estrogens including for example tamoxifen, raloxifene, aromatase inhibiting 4 (5)-imidazoles, 4-hydroxytamoxifen, trioxifene, keoxifene, LY 117018, onapristone, and toremifene (FARESTON®); and anti-androgens such as flutamide, nilutamide, bicalutamide, leuprolide, and goserelin; and pharmaceutically acceptable salts, acids or derivatives of any of the above. Other conventional cytotoxic chemical compounds as those disclosed in Wiemann et al., 1985, in Medical Oncology (Calabresi et aL, eds.), Chapter 10, McMillan Publishing, are also applicable to the methods of the present invention.

### Generation of Bispecific Anti-EGFR/c-Met Antibodies Used in the Methods of the Disclosure

An exemplary bispecific anti-EGFR/c-Met antibody that can be used in the methods of the disclosures is amivantamab. Amivantamab is an IgG1 anti-EGFR/c-Met bispecific antibody described in U.S. Pat. No. 9,593,164, which is incorporated herein by reference in its entirety. Amivantamab is characterized by following amino acid sequences: EGFR binding arm

>(HCDR1, EGFR binding arm)
SEQ ID NO: 1
TYGMH
>(HCDR2, EGFR binding arm)
SEQ ID NO: 2
VIWDDGSYKYYGDSVKG
>(HCDR3, EGFR binding arm)
SEQ ID NO: 3
DGITMVRGVMKDYFDY
>(LCDR1, EGFR binding arm)
SEQ ID NO: 4
RASQDISSALV
>(LCDR2, EGFR binding arm)
SEQ ID NO: 5
DASSLES
>(LCDR3, EGFR binding arm)
SEQ ID NO: 6
QQFNSYPLT
>(HCDR1, c-Met binding arm)
SEQ ID NO: 7
SYGIS
>(HCDR2, c-Met binding arm)
SEQ ID NO: 8
WISAYNGYTNYAQKLQG
>(HCDR3, c-Met binding arm)
SEQ ID NO: 9
DLRGTNYFDY
>(LCDR1, c-Met binding arm)
SEQ ID NO: 10
RASQGISNWLA
>(LCDR2, c-Met binding arm)
SEQ ID NO: 11
AASSLLS
>(LCDR3, c-Met binding arm)
SEQ ID NO: 12
QQANSFPIT
>(VH, EGFR binding arm)
SEQ ID NO: 13
QVQLVESGGGVVQPGRSLRLSCAASGFTFSTYGMHWVRQAPGKGLEWVAVIWDDG
SYKYYGDSVKGRFTISRDNSKNTLYLQMNSLRAEDTAVYYCARDGITMVRGVMKD
YFDYWGQGTLVTVSS
>(VL, EGFR binding arm)
SEQ ID NO: 14
AIQLTQSPSSLSASVGDRVTITCRASQDISSALVWYQQKPGKAPKLLIYDASSLESGVP
SRFSGSESGTDFTLTISSLQPEDFATYYCQQFNSYPLTFGGGTKVEIK
>(VH, c-Met binding arm)
SEQ ID NO: 15
QVQLVQSGAEVKKPGASVKVSCETSGYTFTSYGISWVRQAPGHGLEWMGWISAYN
GYTNYAQKLQGRVTMTTDTSTSTAYMELRSLRSDDTAVYYCARDLRGTNYFDYWG
QGTLVTVSS
>(VL, c-Met binding arm)
SEQ ID NO: 16
DIQMTQSPSSVSASVGDRVTITCRASQGISNWLAWFQHKPGKAPKLLIYAASSLLSGV
PSRFSGSGSGTDFTLTISSLQPEDFATYYCQQANSFPITFGQGTRLEIK
>HC1
SEQ ID NO: 17
QVQLVESGGGVVQPGRSLRLSCAASGFTFSTYGMHWVRQAPGKGLEWVAVIWDDG
SYKYYGDSVKGRFTISRDNSKNTLYLQMNSLRAEDTAVYYCARDGITMVRGVMKD
YFDYWGQGTLVTVSSASTKGPSVFPLAPSSKSTSGGTAALGCLVKDYFPEPVTVSWN
SGALTSGVHTFPAVLQSSGLYSLSSVVTVPSSSLGTQTYICNVNHKPSNTKVDKRVEP
KSCDKTHTCPPCPAPELLGGPSVFLFPPKPKDTLMISRTPEVTCVVVDVSHEDPEVKF
NWYVDGVEVHNAKTKPREEQYNSTYRVVSVLTVLHQDWLNGKEYKCKVSNKALP
APIEKTISKAKGQPREPQVYTLPPSREEMTKNQVSLTCLVKGFYPSDIAVEWESNGQP
ENNYKTTPPVLDSDGSFLLYSKLTVDKSRWQQGNVFSCSVMHEALHNHYTQKSLSL
SPGK
>LC1
SEQ ID NO: 18
AIQLTQSPSSLSASVGDRVTITCRASQDISSALVWYQQKPGKAPKLLIYDASSLESGVP
SRFSGSESGTDFTLTISSLQPEDFATYYCQQFNSYPLTFGGGTKVEIKRTVAAPSVFIFP
PSDEQLKSGTASVVCLLNNFYPREAKVQWKVDNALQSGNSQESVTEQDSKDSTYSL
SSTLTLSKADYEKHKVYACEVTHQGLSSPVTKSFNRGEC
>HC2
SEQ ID NO: 19
QVQLVQSGAEVKKPGASVKVSCETSGYTFTSYGISWVRQAPGHGLEWMGWISAYN
GYTNYAQKLQGRVTMTTDTSTSTAYMELRSLRSDDTAVYYCARDLRGTNYFDYWG
QGTLVTVSSASTKGPSVFPLAPSSKSTSGGTAALGCLVKDYFPEPVTVSWNSGALTS
GVHTFPAVLQSSGLYSLSSVVTVPSSSLGTQTYICNVNHKPSNTKVDKRVEPKSCDK
THTCPPCPAPELLGGPSVFLFPPKPKDTLMISRTPEVTCVVVDVSHEDPEVKFNWYVD
GVEVHNAKTKPREEQYNSTYRVVSVLTVLHQDWLNGKEYKCKVSNKALPAPIEKTI
SKAKGQPREPQVYTLPPSREEMTKNQVSLTCLVKGFYPSDIAVEWESNGQPENNYKT
TPPVLDSDGSFFLYSRLTVDKSRWQQGNVFSCSVMHEALHNHYTQKSLSLSPGK
>LC2
SEQ ID NO: 20
DIQMTQSPSSVSASVGDRVTITCRASQGISNWLAWFQHKPGKAPKLLIYAASSLLSGV
PSRFSGSGSGTDFTLTISSLQPEDFATYYCQQANSFPITFGQGTRLEIKRTVAAPSVFIF
PPSDEQLKSGTASVVCLLNNFYPREAKVQWKVDNALQSGNSQESVTEQDSKDSTYS
LSSTLTLSKADYEKHKVYACEVTHQGLSSPVTKSFNRGEC

In some embodiments, the bispecific anti-EGFR/c-Met antibody comprises a first domain that specifically binds EGFR and a second domain that specifically binds c-Met, wherein the first domain comprises a heavy chain complementarity determining region 1 (HCDR1) of SEQ ID NO: 1, a HCDR2 of SEQ ID NO: 2, a HCDR3 of SEQ ID NO: 3, a light chain complementarity determining region 1 (LCDR1) of SEQ ID NO: 4, a LCDR2 of SEQ ID NO: 5 and a LCDR3 of SEQ ID NO: 6; and the second domain comprises the HCDR1 of SEQ ID NO: 7, the HCDR2 of SEQ ID NO: 8, the HCDR3 of SEQ ID NO: 9, the LCDR1 of SEQ ID NO: 10, the LCDR2 of SEQ ID NO: 11 and the LCDR3 of SEQ ID NO: 12.

In some embodiments, the first domain that specifically binds EGFR comprises a heavy chain variable region (VH) of SEQ ID NO: 13 and a light chain variable region (VL) of SEQ ID NO: 14; and the second domain that specifically binds c-Met comprises the VH of SEQ ID NO: 15 and the VL of SEQ ID NO: 16.

In some embodiments, the bispecific anti-EGFR/c-Met antibody is an IgG1 isotype.

In some embodiments, the bispecific anti-EGFR/c-Met antibody comprises a first heavy chain (HC1) of SEQ ID NO: 17, a first light chain (LC1) of SEQ ID NO: 18, a second heavy chain (HC2) of SEQ ID NO: 19 and a second light chain (LC2) of SEQ ID NO: 20.

In some embodiments, the bispecific anti-EGFR/c-Met antibody comprises a first domain that specifically binds EGFR and a second domain that specifically binds c-Met, wherein the first domain comprises a heavy chain complementarity determining region 1 (HCDR1) comprising SEQ ID NO: 1, a HCDR2 comprising SEQ ID NO: 2, a HCDR3 comprising SEQ ID NO: 3, a light chain complementarity determining region 1 (LCDR1) comprising SEQ ID NO: 4, a LCDR2 comprising SEQ ID NO: 5, and a LCDR3 comprising SEQ ID NO: 6; and the second domain comprises a HCDR1 comprising SEQ ID NO: 7, a HCDR2 comprising SEQ ID NO: 8, a HCDR3 comprising SEQ ID NO: 9, a LCDR1 comprising SEQ ID NO: 10, a LCDR2 comprising SEQ ID NO: 11, and a LCDR3 comprising SEQ ID NO: 12.

In some embodiments, the first domain that specifically binds EGFR comprises a heavy chain variable region (VH) comprising SEQ ID NO: 13 and a light chain variable region (VL) comprising SEQ ID NO: 14; and the second domain that specifically binds c-Met comprises a VH comprising SEQ ID NO: 15 and a VL comprising SEQ ID NO: 16.

In some embodiments, the bispecific anti-EGFR/c-Met antibody is an IgG1 isotype.

In some embodiments, the bispecific anti-EGFR/c-Met antibody comprises a first heavy chain (HC1) comprising SEQ ID NO: 17, a first light chain (LC1) comprising SEQ ID NO: 18, a second heavy chain (HC2) comprising SEQ ID NO: 19, and a second light chain (LC2) comprising SEQ ID NO: 20.

In some embodiments, the bispecific anti-EGFR/c-Met antibody is a biosimilar of amivantamab.

In some embodiments, a non-limiting example of a biosimilar of amivantamab can be found in publicly available Web resource: us.proteogenix_science/product/amivantamab-biosimilar-anti-egfr-me-rccp2-mab-research-grade/.

In some embodiments, a non-limiting example of a biosimilar of amivantamab can be found in publicly available Web resource:

thermofisher_com/antibody/product/Amivantamab-Antibody-Recombinant-Monoclonal/MA5-42260.

In some embodiments, a non-limiting example of a biosimilar of amivantamab can be found in publicly available Web resource: genemedi_net/i/biologics-biosimilar-GMP-Bios-ab-021.

In some embodiments, a non-limiting example of a biosimilar of amivantamab can be found in publicly available Web resource: prosci-inc_com/product/amivantamab-egfr-me-rccp2-research-grade-biosimilar-10-966/.

In some embodiments, a non-limiting example of a biosimilar of amivantamab can be found in publicly available Web resource: antibodysystem_com/product/6201.html.

In some embodiments, a non-limiting example of a biosimilar of amivantamab can be found in publicly available Web resource: biorbyt_com/amivantamab-biosimilar-antibody-orb1140752.html.

In one embodiment, the bispecific anti-EGFR/c-Met antibody comprises one or more Fc silencing mutations.

In one embodiment, the one or more Fc silencing mutations decrease affinity to Fcγ receptors.

In one embodiment, the one or more Fc silencing mutations comprise V234A/G237A/P238S/H268A/V309L/A330S/P331S.

In one embodiment, the bispecific anti-EGFR/c-Met antibody comprises a biantennary glycan structure with a fucose content between about 1% to about 15%. Antibodies with reduced fucose content can be made using different methods reported to lead to the successful expression of relatively high defucosylated antibodies bearing the biantennary complex-type of Fc oligosaccharides such as control of culture osmolality (Konno et al., Cytotechnology 64(:249-65, 2012), application of a variant CHO line Lec13 as the host cell line (Shields et al., J Biol Chem 277:26733-26740, 2002), application of a variant CHO line EB66 as the host cell line (Olivier et al., MAbs; 2(4), 2010; Epub ahead of print; PMID: 20562582), application of a rat hybridoma cell line YB2/0 as the host cell line (Shinkawa et al., J Biol Chem 278:3466-3473, 2003), introduction of small interfering RNA specifically against the α-1,6-fucosyltrasferase (FUT8) gene (Mori et al., Biotechnol Bioeng88: 901-908, 2004), or coexpression of β-1,4-N-acetylglucosaminyltransferase III and Golgi α-mannosidase II or a potent alpha-mannosidase I inhibitor, kifunensine (Ferrara et al., J Biol Chem281: 5032-5036, 2006, Ferrara et al., Biotechnol Bioeng 93:851-861, 2006; Xhou et al., Biotechnol Bioeng 99:652-65, 2008). In general, lowering fucose content in the glycan of the antibodies potentiates antibody-mediated cellular cytotoxicity (ADCC).

Other bispecific anti-EGFR/c-Met antibodies publicly available may also be used in the methods of the disclosure as long as they demonstrate similar characteristics when compared to amivantamab as described in U.S. Pat. No. 9,593,164. Bispecific anti-EGFR/c-Met antibodies that may be used in the methods of the disclosure may also be generated by combining EGFR binding VH/VL domains and c-Met binding VH/VL domains that are publicly available and testing the resulting bispecific antibodies for their characteristics as described in U.S. Pat. No. 9,593,164.

Bispecific anti-EGFR/c-Met antibodies used in the methods of the disclosure may be generated for example using Fab arm exchange (or half molecule exchange) between two monospecific bivalent antibodies by introducing substitutions at the heavy chain CH3 interface in each half molecule to favor heterodimer formation of two antibody half molecules having distinct specificity either in vitro in cell-free environment or using co-expression. The Fab arm exchange reaction is the result of a disulfide-bond isomerization reaction and dissociation-association of CH3 domains. The heavy chain disulfide bonds in the hinge regions of the parental monospecific antibodies are reduced. The resulting free cysteines of one of the parental monospecific antibodies form an inter heavy-chain disulfide bond with cysteine residues of a second parental monospecific antibody molecule and simultaneously CH3 domains of the parental antibodies release and reform by dissociation-association. The CH3 domains of the Fab arms may be engineered to favor heterodimerization over homodimerization. The resulting product is a bispecific antibody having two Fab arms or half molecules which each bind a distinct epitope, i.e., an epitope on EGFR and an epitope on c-Met. For example, the bispecific antibodies of the invention may be generated using the technology described in Int. Pat. Publ. No. WO2011/131746. Mutations F405L in one heavy chain and K409R in the other heavy chain may be used in case of IgG1 antibodies. For IgG2 antibodies, a wild-type IgG2 and a IgG2 antibody with F405L and R409K substitutions may be used. For IgG4 antibodies, a wild-type IgG4 and a IgG4 antibody with F405L and R409K substitutions may be used. To generate bispecific antibodies, first monospecific bivalent antibody and the second monospecific bivalent antibody are engineered to have the aforementioned mutation in the Fc region, the antibodies are incubated together under reducing conditions sufficient to allow the cysteines in the hinge region to undergo disulfide bond isomerization; thereby generating the bispecific antibody by Fab arm exchange. The incubation conditions may optimally be restored to non-reducing. Exemplary reducing agents that may be used are 2-mercaptoethylamine (2-MEA), dithiothreitol (DTT), dithioerythritol (DTE), glutathione, tris(2-carboxyethyl)phosphine (TCEP), L-cysteine and beta-mercaptoethanol. For example, incubation for at least 90 min at a temperature of at least 20 °C in the presence of at least 25 mM 2-MEA or in the presence of at least 0.5 mM dithiothreitol at a pH of from 5-8, for example at pH of 7.0 or at pH of 7.4 may be used.

Bispecific anti-EGFR/c-Met antibodies used in the methods of the disclosure may also be generated using designs such as the Knob-in-Hole (Genentech), CrossMAbs (Roche) and the electrostatically-matched (Chugai, Amgen, NovoNordisk, Oncomed), the LUZ-Y (Genentech), the Strand Exchange Engineered Domain body (SEEDbody)(EMD Serono), and the Biclonic (Merus).

In the “knob-in-hole” strategy (see, e.g., Intl. Publ. No. WO 2006/028936) select amino acids forming the interface of the CH3 domains in human IgG can be mutated at positions affecting CH3 domain interactions to promote heterodimer formation. An amino acid with a small side chain (hole) is introduced into a heavy chain of an antibody specifically binding a first antigen and an amino acid with a large side chain (knob) is introduced into a heavy chain of an antibody specifically binding a second antigen. After co-expression of the two antibodies, a heterodimer is formed as a result of the preferential interaction of the heavy chain with a “hole” with the heavy chain with a “knob”. Exemplary CH3 substitution pairs forming a knob and a hole are (expressed as modified position in the first CH3 domain of the first heavy chain/modified position in the second CH3 domain of the second heavy chain): T366Y/F405A, T366W/F405W, F405W/Y407A, T394W/Y407T, T394S/Y407A, T366W/T394S, F405W/T394S and T366W/T366S_L368A_Y407V.

CrossMAb technology, in addition to utilizing the “knob-in-hole” strategy to promoter Fab arm exchange utilizes CH1/CL domain swaps in one half arm to ensure correct light chain pairing of the resulting bispecific antibody (see e.g., U.S. Pat. No. 8,242,247).

Other cross-over strategies may be used to generate full length bispecific antibodies of the invention by exchanging variable or constant, or both domains between the heavy chain and the light chain or within the heavy chain in the bispecific antibodies, either in one or both arms. These exchanges include for example VH-CH1 with VL-CL, VH with VL, CH3 with CL and CH3 with CH1 as described in Int. Patent Publ. Nos. WO2009/080254, WO2009/080251, WO2009/018386, and WO2009/080252.

Other strategies such as promoting heavy chain heterodimerization using electrostatic interactions by substituting positively charged residues at one CH3 surface and negatively charged residues at a second CH3 surface may be used, as described in US Patent Publ. No. US2010/0015133; US Patent Publ. No. US2009/0182127; US Patent Publ. No. US2010/028637 or US Patent Publ. No. US2011/0123532. In other strategies, heterodimerization may be promoted by following substitutions (expressed as modified positions in the first CH3 domain of the first heavy chain/modified position in the second CH3 domain of the second heavy chain): L351Y_F405A_Y407V/T394W, T366I_K392M_T394W/F405A_Y407V, T366L_K392M_T394W/F405A_Y407V, L351Y_Y407A/T366A_K409F, L351Y_Y407A/T366V_K409F, Y407A/T366A_K409F, or T350V_L351Y_F405A_Y407V/T350V_T366L_K392L_T394W as described in U.S. Patent Publ. No. US2012/0149876 or U.S. Patent Publ. No. US2013/0195849.

SEEDbody technology may be utilized to generate bispecific antibodies of the invention. SEEDbodies have, in their constant domains, select IgG residues substituted with IgA residues to promote heterodimerization as described in U.S. Patent No. US20070287170.

Mutations are typically made at the DNA level to a molecule such as the constant domain of the antibody using standard methods.

### Exemplary Embodiments

Provided below is a list of exemplary embodiments:
1a. A method of improving median progression free survival (PFS) in a population of subjects with locally advanced or metastatic non-small cell lung cancer (NSCLC) harboring one or more epidermal growth factor receptor (EGFR) mutations who are treatment-naïve, the method comprising administering to the population of subjects a combination therapy comprising:
(i) a therapeutically effective amount of a bispecific anti-EGFR/c-Met antibody, and
(ii) a therapeutically effective amount of lazertinib, or a pharmaceutically acceptable salt or hydrate thereof, and
wherein the improvement in median PFS is relative to median PFS of a reference population of subjects with NSCLC harboring one or more EGFR mutations who are treatment-naive, the reference population having been administered osimertinib or lazertinib without the bispecific anti-EGFR/c-Met antibody.
2a. A method of improving overall survival (OS) in a subject or a population of subjects with locally advanced or metastatic non-small cell lung cancer (NSCLC) harboring one or more epidermal growth factor receptor (EGFR) mutations who are treatment-naïve, the method comprising administering to the subject or the population of subjects a combination therapy comprising:
(i) a therapeutically effective amount of a bispecific anti-EGFR/c-Met antibody, and
(ii) a therapeutically effective amount of lazertinib, or a pharmaceutically acceptable salt or hydrate thereof, and
wherein the improvement in OS is relative to OS of a reference subject or a reference population of subjects with NSCLC harboring one or more EGFR mutations who are treatment-naive, the reference subject or the reference population of subjects having been administered osimertinib or lazertinib without the bispecific anti-EGFR/c-Met antibody.
3a. The method of embodiment 1a or 2a, wherein the lazertinib, or pharmaceutically acceptable salt or hydrate thereof, is lazertinib mesylate.
4a. The method of embodiment 1a or 2a, wherein the lazertinib, or pharmaceutically acceptable salt or hydrate thereof, is lazertinib mesylate monohydrate.
5a. The method of any one of embodiments 1a-4a, wherein the one or more EGFR mutations comprise one or more exon 19 deletions, or exon 21 L858R substitution, or any combination thereof.
6a. The method of any one of embodiments 1a-4a, wherein the one or more EGFR mutations comprise one or more exon 19 deletions.
7a. The method of any one of embodiments 1a-4a, wherein the one or more EGFR mutations comprise exon 21 L858R substitution.
8a. The method of any one of embodiments 1a-7a, wherein the subject has newly diagnosed, locally advanced or metastatic NSCLC that is not amenable to curative therapy including surgical resection or chemoradiation.
9a. The method of embodiment 8a, wherein the curative therapy includes surgical resection or chemoradiation.
10a. The method of any one of embodiments 1a-9a, wherein the method comprises administering the lazertinib, or pharmaceutically acceptable salt or hydrate thereof, in an amount of about 80 mg to about 320 mg orally once daily.
11a. The method of any one of embodiments 1a-10a, wherein the method comprises administering the lazertinib, or pharmaceutically acceptable salt or hydrate thereof, in an amount of about 240 mg orally once daily.
12a. The method of any one of embodiments 1a-11a, wherein the method elicits a clinical response in the subject according to RECIST v1.1 criteria.
13a. The method of any one of embodiments 1a-12a, wherein the method achieves a partial response or better in the subject according to RECIST v1.1 criteria.
14a. The method of any one of embodiments 1a-13a, wherein the clinical response comprises a median duration of response (DOR) of at least 25 months.
15a. The method of any one of embodiments 1a-14a, wherein the subject is progression-free after at least 11 months.
16a. The method of any one of embodiments 1a-15a, wherein the subject is progression-free after at least 23 months.
17a. The method of any one of embodiments 1a-16a, wherein the method achieves a PFS rate of 87% at 6 months, 73% at 12 months, 60% at 18 months, 48% at 24 months and 41% at 30 months in a population of the treatment naïve subjects diagnosed with locally advanced or metastatic NSCLC harboring one or more epidermal growth factor receptor (EGFR) mutations.
18a. The method of any one of embodiments 1a-17a, wherein the bispecific anti-EGFR/c-Met antibody comprises a first domain that specifically binds EGFR and a second domain that specifically binds c-Met, wherein the first domain comprises a heavy chain complementarity determining region 1 (HCDR1) comprising SEQ ID NO: 1, a HCDR2 comprising SEQ ID NO: 2, a HCDR3 comprising SEQ ID NO: 3, a light chain complementarity determining region 1 (LCDR1) comprising SEQ ID NO: 4, a LCDR2 comprising SEQ ID NO: 5, and a LCDR3 comprising SEQ ID NO: 6, and wherein the second domain that binds c-Met comprises a HCDR1 comprising SEQ ID NO: 7, a HCDR2 comprising SEQ ID NO: 8, a HCDR3 comprising SEQ ID NO: 9, a LCDR1 comprising SEQ ID NO: 10, a LCDR2 comprising SEQ ID NO: 11, and a LCDR3 comprising SEQ ID NO: 12.
19a. The method of embodiment 18a, wherein the first domain that specifically binds EGFR comprises a heavy chain variable region (VH) comprising SEQ ID NO: 13 and a light chain variable region (VL) comprising SEQ ID NO: 14, and the second domain that specifically binds c-Met comprises a VH comprising SEQ ID NO: 15 and a VL comprising SEQ ID NO: 16.
20a. The method of embodiment 18a or 19a, wherein the bispecific anti-EGFR/c-Met antibody is an IgG1 isotype.
21a. The method of any one of embodiments 1a-20a, wherein the bispecific anti-EGFR/c-Met antibody comprises a first heavy chain (HC1) comprising SEQ ID NO: 17, a first light chain (LC1) comprising SEQ ID NO: 18, a second heavy chain (HC2) comprising SEQ ID NO: 19, and a second light chain (LC2) comprising SEQ ID NO: 20.
22a. The method of any one of embodiments 1a-21a, wherein the bispecific anti-EGFR/c-Met antibody comprises a biantennary glycan structure with a fucose content of about between 1% to about 15%.
23a. The method of any one of embodiments 1a-22a, wherein the bispecific anti-EGFR/c-Met antibody is administered intravenously to the subject.
24a. The method of embodiment 23a, wherein the bispecific anti-EGFR/c-Met antibody is administered at a dose of between about 140 mg to about 2240 mg.
25a. The method of embodiment 24a, wherein the bispecific anti-EGFR/c-Met antibody is administered at a dose of about 700 mg, about 750 mg, about 800 mg, about 850 mg, about 900 mg, about 950 mg, about 1000 mg, about 1050 mg, about 1100 mg, about 1150 mg, about 1200 mg, about 1250 mg, about 1300 mg, about 1350 mg, about 1400 mg, about 1575 mg, about 1600 mg, about 2100 mg, or about 2240 mg.
26a. The method of embodiment 25a, wherein the bispecific anti-EGFR/c-Met antibody is administered at a dose of 1050 mg if the subject has a body weight of less than 80 kg.
27a. The method of embodiment 26a, wherein the bispecific anti-EGFR/c-Met antibody is administered at a dose of 1400 mg if the subject has a body weight of greater than or equal to 80 kg.
28a. The method of any one of embodiments 1a-22a, wherein the bispecific anti-EGFR/c-Met antibody is administered subcutaneously or intradermally to the subject.
29a. The method of embodiment 28a, wherein the bispecific anti-EGFR/c-Met antibody is administered subcutaneously or intradermally at a dose sufficient to achieve a therapeutic effect in the subject.
30a. The method of any one of embodiments 1a-29a, wherein the bispecific anti-EGFR/c-Met antibody is administered twice a week, once a week, once in two weeks, once in three weeks or once in four weeks.
31a. The method of any one of embodiments 1-30, wherein the subject or population of subjects has a baseline brain metastasis, baseline liver metastasis, TP53 co-mutation, detectable baseline EGFRm ctDNA, or is without EGFRm ctDNA clearance at C3D1

Provided below is a list of further exemplary embodiments:
1b. A method of treating non-small cell lung cancer (NSCLC) in a subject in need thereof, comprising administering to the subject a combination therapy comprising
(i) a therapeutically effective amount of a bispecific anti-EGFR/c-Met antibody; and
(ii) a therapeutically effective amount of lazertinib, or a pharmaceutically acceptable salt or hydrate thereof,
wherein the subject has been diagnosed with locally advanced or metastatic NSCLC harboring one or more epidermal growth factor receptor (EGFR) mutations, and
wherein the subject is treatment naïve.
2b. The method of embodiment 1b, herein the lazertinib, or pharmaceutically acceptable salt or hydrate thereof, is lazertinib mesylate.
3b. The method of embodiment 1b, wherein the lazertinib, or pharmaceutically acceptable salt or hydrate thereof, is lazertinib mesylate monohydrate.
4b. The method of any one of embodiments 1b-3b, wherein the one or more EGFR mutations comprise one or more exon 19 deletions, or exon 21 L858R substitution, or any combination thereof.
5b. The method of any one of embodiments 1b-3b, wherein the one or more EGFR mutations comprise one or more exon 19 deletions.
6b. The method of any one of embodiments 1b-3b, wherein the one or more EGFR mutations comprise exon 21 L858R substitution.
7b. The method of any one of embodiments 1b-6b, wherein the subject has newly diagnosed, locally advanced or metastatic NSCLC that is treatment naïve and not amenable to curative therapy including surgical resection or chemoradiation.
8b. The method of embodiment 7b, wherein the curative therapy includes surgical resection or chemoradiation.
9b. The method of any one of embodiments 1b-8b, wherein the method comprises administering the lazertinib, or pharmaceutically acceptable salt or hydrate thereof, in an amount of about 80 mg to about 320 mg orally once daily.
10b. The method of any one of embodiments 1b-9b, wherein the method comprises administering the lazertinib, or pharmaceutically acceptable salt or hydrate thereof, in an amount of about 240 mg orally once daily.
11b. The method of any one of embodiments 1b-10b, wherein the method elicits a clinical response in the subject according to RECIST v1.1 criteria.
12b. The method of any one of embodiments 1b-11b, wherein the method achieves a partial response or better in the subject according to RECIST v1.1 criteria.
13b. The method of any one of embodiments 1b-12b, wherein the clinical response comprises a duration of response (DOR) of at least 1 year, or at least 2 years, or at least 3 years.
14b. The method of any one of embodiments 1b-13b, wherein the subject is progression-free after at least 20 months.
15b. The method of any one of embodiments 1b-13b, wherein the subject is progression-free after at least 30 months.
16b. The method of any one of embodiments 1b-15b, wherein the method achieves a PFS rate of 85% at 12 months, 65% at 24 months, and 51% at 36 months in a population of the treatment naïve subjects diagnosed with locally advanced or metastatic NSCLC harboring one or more epidermal growth factor receptor (EGFR) mutations.
17b. The method of any one of embodiments 1b-16b, wherein the bispecific anti-EGFR/c-Met antibody comprises a first domain that specifically binds EGFR and a second domain that specifically binds c-Met, wherein the first domain comprises a heavy chain complementarity determining region 1 (HCDR1) of SEQ ID NO: 1, a HCDR2 of SEQ ID NO: 2, a HCDR3 of SEQ ID NO: 3, a light chain complementarity determining region 1 (LCDR1) of SEQ ID NO: 4, a LCDR2 of SEQ ID NO: 5 and a LCDR3 of SEQ ID NO: 6, and wherein the second domain that binds c-Met comprises the HCDR1 of SEQ ID NO: 7, the HCDR2 of SEQ ID NO: 8, the HCDR3 of SEQ ID NO: 9, the LCDR1 of SEQ ID NO: 10, the LCDR2 of SEQ ID NO: 11 and the LCDR3 of SEQ ID NO: 12.
18b. The method of embodiment 17b, wherein the first domain that specifically binds EGFR comprises a heavy chain variable region (VH) of SEQ ID NO: 13 and a light chain variable region (VL) of SEQ ID NO: 14, and the second domain that specifically binds c-Met comprises the VH of SEQ ID NO: 15 and the VL of SEQ ID NO: 16.
19b. The method of embodiment 17b or 18b, wherein the bispecific anti-EGFR/c-Met antibody is an IgG1 isotype.
20b. The method of any one of embodiments 1b-19b, wherein the bispecific anti-EGFR/c-Met antibody comprises a first heavy chain (HC1) of SEQ ID NO: 17, a first light chain (LC1) of SEQ ID NO: 18, a second heavy chain (HC2) of SEQ ID NO: 19 and a second light chain (LC2) of SEQ ID NO: 20.
21b. The method of any one of embodiments 1b-20b, wherein the bispecific anti-EGFR/c-Met antibody comprises a biantennary glycan structure with a fucose content of about between 1% to about 15%.
22b. The method of any one of embodiments 1b-21b, wherein the bispecific anti-EGFR/c-Met antibody is administered intravenously to the subject.
23b. The method of embodiment 22b, wherein the bispecific anti-EGFR/c-Met antibody is administered at a dose of between about 140 mg to about 2240 mg.
24b. The method of embodiment 23b, wherein the bispecific anti-EGFR/c-Met antibody is administered at a dose of about 700 mg, about 750 mg, about 800 mg, about 850 mg, 900 mg, 950 mg, 1000 mg, 1050 mg, 1100 mg, 1150 mg, 1200 mg, 1250 mg, 1300 mg, 1350 mg, 1400 mg, 1575 mg, 1600 mg, 2100 mg, or 2240 mg.
25b. The method of embodiment 24b, wherein the bispecific anti-EGFR/c-Met antibody is administered at a dose of 1050 mg if the subject has a body weight of less than 80 kg.
26b. The method of embodiment 25b, wherein the bispecific anti-EGFR/c-Met antibody is administered at a dose of 1400 mg if the subject has a body weight of greater than or equal to 80 kg.
27b. The method of any one of embodiments 1b-21b, wherein the bispecific anti-EGFR/c-Met antibody is administered subcutaneously or intradermally to the subject.
28b. The method of embodiment 27, wherein the bispecific anti-EGFR/c-Met antibody is administered subcutaneously or intradermally at a dose sufficient to achieve a therapeutic effect in the subject.
29b. The method of any one of embodiments 1b-28b, wherein the bispecific anti-EGFR/c-Met antibody is administered twice a week, once a week, once in two weeks, once in three weeks or once in four weeks.

### EXAMPLES

The following examples are provided to further describe some of the embodiments disclosed herein. The examples are intended to illustrate, not to limit, the disclosed embodiments.

### Example 1. CHRYSALIS Clinical Study

CHRYSALIS (NCT02609776) is a Phase 1, first-in-human, open-label dose escalation and expansion study of amivantamab in combination with lazertinib (Study 61186372EDI1001, known as EDI1001). For the amivantamab and lazertinib dose escalation/expansion, enrolled subjects must have been diagnosed with EGFR Exon 19del or exon 21 L858R activating mutation and be treatment-naïve for metastatic disease, without access to third generation TKI in the front-line setting or have progressed after front-line treatment with first or second generation or have been treated with a third generation TKI in either the front-line or second line setting.

The initial dose cohort combined amivantamab 700/1050 mg (i.e., 700 mg in subjects weighing <80 kg and 1050 mg in subjects weighing ≥80 kg) and lazertinib 240 mg. The subsequent dose cohort combined each agent at its RP2D from monotherapy studies, namely amivantamab 1050/1400 mg and lazertinib 240 mg. Both dose cohorts were cleared without identification of a dose-limiting toxicity and additional subjects were enrolled. Pharmacokinetic (PK) data demonstrated a lack of drug-drug interaction, as the PK profile of each drug when administered in combination was consistent with the PK profile of each drug when administered as a monotherapy. Thus, RP2D was the recommended monotherapy of each molecule: amivantamab, 1050 mg (<80 kg)/1400 mg (≥80 kg), intravenous dosing, C1 once weekly, then every 2 weeks and 240 mg lazertinib oral daily dosing.

To further characterize safety, tolerability and preliminary efficacy of amivantamab and Lazertinib at RP2D, an additional expansion cohort (Cohort E) was initiated. Forty-five subjects were enrolled who met the following criteria: diagnosed with Exon 19del or exon 21 L858R activating EGFR mutation who progressed after first or second-line treatment with a third generation TKI and chemotherapy-naïve. Limited previous treatment with a platinum-based chemotherapy regimen in metastatic setting was permitted if it did not exceed 2 cycles, and was administered prior to the initiation of the first EGFR TKI.

### DESCRIPTION

The purpose of the study is to evaluate the safety, pharmacokinetics, and preliminary efficacy of amivantamab as a monotherapy and in combination with lazertinib, and to determine the recommended Phase 2 dose (RP2D) (monotherapy), recommended Phase 2 combination dose (RP2CD) (combination therapy), and to determine recommended Phase 2 Dose (RP2Q3W) with combination chemotherapy (amivantamab in combination with standard of care carboplatin and pemetrexed) in 21 day treatment cycle for participants with advanced non-small cell lung cancer (NSCLC).

This open label (all participants know the identity of the study drug), multicenter (more than one study site), first-in-human study consists of 2 parts. Part 1 is a Amivantamab Monotherapy and Combination Dose Escalations and Part 2 Amivantamab Monotherapy and Combination Dose Expansions. In Part 1, participants with evaluable NSCLC will be enrolled into cohorts at increasing dose levels of Amivantamab monotherapy, the RP2CD of the Amivantamab and lazertinib combination which will be administered in 28 day treatment cycles, and RP2Q3W of Amivantamab in combination with standard of care carboplatin and pemetrexed (chemotherapy combination) which will be administered in 21 day treatment cycles. The dose will be escalated until the maximum tolerated dose (MTD, or maximum administered dose (MAD), if no MTD is found) is reached. Part 1 will follow a traditional 3+3 design. At each dose level, 3 participants will complete Cycle 1. If no dose limiting toxicity (DLT) occurs in these 3 participants, then escalation will continue in a new cohort of 3 participants. Data from Part 1 will be used to determine one or more RP2D regimen(s). In Part 2, participants with documented epidermal growth factor receptor (EGFR) mutations and measurable disease, whose disease has progressed after previous treatment will be enrolled and receive Amivantamab at the RP2D determined in Part 1 as a monotherapy at the RP2D regimen(s), or in combination with lazertinib at the RP2CD regimen. For both parts, the study consists of following periods: an optional pre-Screening period; a Screening period (up to 28 days prior to the first dose of study drug); a Treatment period (first dose of study drug until 30 (+7) days after the last dose of study drug or prior to starting any subsequent anti-cancer treatment, whichever comes first); and a Follow Up period (approximately 6 months). All participants will be followed for survival in the post-treatment follow-up period until the end of study and safety will be monitored throughout the study.

### Study Design

Study Type: Interventional (Clinical Trial).
Estimated Enrollment: 780 participants.
Allocation: Non-Randomized.
Intervention Model: Parallel Assignment.
Masking: None (Open Label).
Primary Purpose: Treatment.
Official Title: A Phase 1, First-in-Human, Open-Label, Dose Escalation Study of JNJ-61186372, a Human Bispecific EGFR and cMet Antibody, in Subjects With Advanced Non-Small Cell Lung Cancer.

### Arms and Interventions

TABLE 1

| Arm | Intervention/treatment |
|---|---|
| Experimental: Part 1: Amivantamab | Drug: Amivantamab |
| Monotherapy + Combination Dose | The first cohort of participants will receive |
| Escalations | IV infusions of Amivantamab 140 mg as |
| The first cohort of participants will receive | monotherapy. Each subsequent cohort will |
| intravenous (IV) infusions of Amivantamab | receive IV infusions of Amivantamab at |
| 140 milligram (mg) as monotherapy. Each | increased dose level until maximum |
| subsequent cohort will receive IV infusions | tolerated dose is reached or all planned |
| of Amivantamab at increased dose level. | doses are administered. Participants will |
| Dose escalation will continue until | receive lazertinib and Amivantamab at |
| maximum tolerated dose is reached or all | predefined dose levels, based upon observed |
| planned doses are administered. Participants | safety and protocol defined criteria. The |
| will receive IV infusion of Amivantamab | duration of each treatment cycle is 28 days. |
| once weekly during cycle 1 and once every | In Chemotherapy Combination Cohort, |
| 2 weeks during subsequent cycles (duration | participants will receive Amivantamab, |
| of each treatment cycle is 28 days). | administered on a 21-day cycle, in |
| Participants will receive lazertinib and | combination with the administration of |
| Amivantamab on Cycle 1 Day 1 (C1D1) | standard of care carboplatin and |
| prior to initiation of Amivantamab (C1D1) | pemetrexed. |
| at predefined dose levels, based upon | Other Name: JNJ-61186372 |
| observed safety and protocol defined | Drug: Lazertinib |
| criteria. Lazertinib will be administered | Lazertinib will be administered in |
| daily thereafter, on 28-day Amivantamab | combination with Amivantamab at |
| treatment cycle. In Chemotherapy | predefined dose levels, based upon observed |
| Combination Cohort, participants will | safety and protocol defined criteria. |
| receive Amivantamab, administered on a | Lazertinib will be administered daily on the |
| 21-day cycle, in combination with standard | 28-day Amivantamab treatment cycle. |
| of care carboplatin and pemetrexed. | Other Names: |
| | JNJ-73841937 |
| | YH25448 |
| | Drug: Carboplatin |
| | Participants will receive carboplatin in |
| | combination with pemetrexed and |
| | Amivantamab as an IV infusion on 21-day |
| | treatment cycle in Part 1 Chemotherapy |
| | Combination Cohort only. |
| | Drug: Pemetrexed |
| | Participants will receive pemetrexed in |
| | combination with carboplatin and |
| | Amivantamab as an IV infusion on 21-day |
| | treatment cycle in Part 1 Chemotherapy |
| | Combination Cohort only. |
| Experimental: Part 2: Amivantamab | Drug: Amivantamab |
| Monotherapy + Combination Dose | Participants will receive IV infusion of |
| Expansion | Amivantamab as monotherapy at RP2D |
| Participants will receive IV infusion of | regimen or in combination lazertinib at |
| Amivantamab as monotherapy at Phase 2 | RP2CD regimen as determined in Part 1. |
| dose (RP2D) regimen or in combination | Other Name: JNJ-61186372 |
| lazertinib at the recommended Phase 2 | Drug: Lazertinib |
| combination dose (RP2CD) regimen as | Lazertinib will be administered in |
| determined in Part 1. The purpose of dose | combination with Amivantamab at |
| expansion is to further evaluate safety, | predefined dose levels, based upon observed |
| tolerability, pharmacokinetic, and to assess | safety and protocol defined criteria. |
| preliminary efficacy in monotherapy and | Lazertinib will be administered daily on the |
| combination therapy cohorts. | 28-day Amivantamab treatment cycle. |
| | Other Names: |
| | JNJ-73841937 |
| | YH25448 |

### Outcome Measures

### Primary Outcome Measures

1. Part 1: Number of Participants With Dose Limiting Toxicity (DLT) [Time Frame: Up to Day 28]. The Dose Limiting Toxicity (DLT) is based on drug related adverse events and includes unacceptable hematologic toxicity, non-hematologic toxicity of Grade 3 or higher, or elevations in hepatic enzymes suggestive of drug-induced liver injury.
2. Part 2: Number of Participants With Adverse Events (AEs) and Serious AEs [Time Frame: Screening up to follow-up (30 [+7] days after the last dose)]. An adverse event (AE) is any untoward medical occurrence in a participant who received study drug without regard to possibility of causal relationship. A serious adverse event (SAE) is an AE resulting in any of the following outcomes or deemed significant for any other reason: death; initial or prolonged inpatient hospitalization; life-threatening experience (immediate risk of dying); persistent or significant disability/incapacity; congenital anomaly.
3. Part 2: Overall Response Rate (ORR) [Time Frame: Up to End of Treatment Follow (EOT) Up Period (30 [+7] days after the last dose)]. Overall response rate (ORR) is defined as the percentage of participants who achieve either a CR or PR as per Response Evaluation Criteria In Solid Tumors Criteria (RECIST v1.1). CR: disappearance of all target lesions and non-target lesions. All lymph nodes must be non-pathological in size (<10 mm short axis) and normalisation of tumour marker levels; PR: at least a 30% decrease in the sum of diameters of target lesions, taking as reference the baseline sum diameters and Persistence of one or more non-target lesion(s) and/or maintenance of tumour marker level above the normal limits.
4. Part 2: Duration of Response (DOR) [Time Frame: Up to EOT Follow Up Period (30 [+7] days after the last dose)]. DOR will be calculated as time from initial response of CR (disappearance of all target lesions and non-target lesions. All lymph nodes must be non-pathological in size ([<] 10 [mm] short axis) and normalisation of tumour marker levels) or PR (at least a 30 [%] decrease in the sum of diameters of target lesions, taking as reference the baseline sum diameters and persistence of one or more non-target lesion(s) and/or maintenance of tumour marker level above the normal limits or durable stable disease (neither sufficient shrinkage to qualify for PR nor sufficient increase to qualify for progressive disease (PD), taking as reference the smallest sum diameters while on study and persistence of one or more non-target lesion(s) and/or maintenance of tumour marker level above the normal limits) to progressive disease (PD) or death due to underlying disease, whichever comes first, only for participants who achieve CR or PR.
5. Part 2: Percentage of Participants With Clinical Benefit [Time Frame: Up to EOT Follow Up Period (30 [+7] days after the last dose)]. Clinical benefit rate is defined as the percentage of participants achieving complete response (CR): disappearance of all target lesions and non-target lesions. All lymph nodes must be non-pathological in size (less than [<] 10 millimeter [mm] short axis) and normalisation of tumour marker levels or partial response (PR): at least a 30 percent (%) decrease in the sum of diameters of target lesions, taking as reference the baseline sum diameters and persistence of one or more non-target lesion(s) and/or maintenance of tumour marker level above the normal limits or durable stable disease (neither sufficient shrinkage to qualify for PR nor sufficient increase to qualify for progressive disease (PD), taking as reference the smallest sum diameters while on study and persistence of one or more non-target lesion(s) and/or maintenance of tumour marker level above the normal limits.
6. Trough Serum Concentration (Ctrough) of Amivantamab [Time Frame: Up to EOT (30 days after last dose)]. Ctrough is the observed serum concentration immediately prior to the next administration.
7. Area Under the Curve From Time Zero to End of Dosing Interval (AUCtau) of Amivantamab [Time Frame: Up to EOT (30 days after last dose)]. The AUCtau is the area under the serum concentration-time curve during a dose interval time period (tau).

### Secondary Outcome Measures

1. Maximum Serum Concentration (Cmax) of Amivantamab [Time Frame: Cycle 1 Day 1: predose through end of infusion (EOT) or Follow Up (approximately 16 months) (each cycle is of 28 days)]. The Cmax is the maximum observed serum concentration of Amivantamab.
2. Time to Reach Maximum Observed Serum Concentration (Tmax) of Amivantamab [Time Frame: Cycle 1 Day 1: predose through EOT or Follow Up (approximately 16 months)]. The Tmax is defined as time to reach maximum observed serum concentration of Amivantamab.
3. Area Under the Serum Concentration-Time Curve From t1 to t2 Time (AUC [t1-t2]) of Amivantamab [Time Frame: Cycle 1 Day 1: predose through EOT or Follow Up (approximately 16 months)]. The AUC (t1-t2) is the area under the serum Amivantamab concentration-time curve from time t1 to t2.
4. Area Under the Curve From Time Zero to End of Dosing Interval (AUCtau) of Amivantamab [Time Frame: Cycle 1 Day 1: predose through EOT or Follow Up (approximately 16 months)]. The AUCtau is the area under the serum concentration-time curve during a dose interval time period (tau).
5. Trough Serum Concentration (Ctrough) of Amivantamab [Time Frame: Cycle 1 Day 1: predose through EOT or Follow Up (approximately 16 months)]. The Ctrough is the observed serum concentration immediately prior to the next administration.
6. Maximum Serum Concentration (Cmax) of Lazertinib [Time Frame: Cycle 1 Day 1: predose through EOT (30 [+7] days after last dose [Cycle 4 Day 15]) (each cycle is of 28 days)]. Cmax is the maximum observed serum concentration of lazertinib.
7. Time to Reach Maximum Observed Serum Concentration (Tmax) of Lazertinib [Time Frame: Cycle 1 Day 1: predose through EOT (30 [+7] days after last dose [Cycle 4 Day 15]) (each cycle is of 28 days)]. Tmax is defined as time to reach maximum observed serum concentration of lazertinib.
8. Trough Serum Concentration (Ctrough) of Lazertinib [Time Frame: Cycle 1 Day 1: predose through EOT (30 [+7] days after last dose [Cycle 4 Day 15]) (each cycle is of 28 days)]. Ctrough is the observed serum concentration immediately prior to the next administration.
9. Accumulation ratio (R) of Amivantamab [Time Frame: Cycle 1 Day 1: predose through EOT or Follow Up (approximately 16 months)]. The R is the accumulation ratio calculated as Cmax or AUC after multiple doses divided by Cmax or AUC after the first dose, respectively.
10. Number of Participants With Anti-Drug Antibodies (ADA) [Time Frame: Cycle 1 Day 1: predose through EOT or Follow Up (approximately 16 months)]. Serum levels of antibodies to Amivantamab for evaluation of potential immunogenicity.
11. Progression-Free Survival (PFS) [Time Frame: Up to End of Treatment Follow Up Period (30 [+7] days after the last dose)]. PFS is defined as the time from first infusion of study drug to PD or death due to any cause.
12. Time to Treatment Failure (TTF) [Time Frame: Up to End of Treatment Follow Up Period (30 [+7] days after the last dose)]. TTF is defined as the time from the first infusion of the study drug to discontinuation of treatment for any reason, including disease progression, treatment toxicity, death, and will be utilized to capture clinical benefit for patients continuing treatment beyond RECIST v1.1 defined disease progression.
13. Overall Survival (OS) [Time Frame: Up to End of Treatment Follow Up Period (30 [+7] days after the last dose)]. OS is defined as the time from first infusion of study drug to death due to any cause.

### Eligibility Criteria.

Ages Eligible for Study: 18 Years and older (Adult, Older Adult).
Sexes Eligible for Study: All.
Accepts Healthy Volunteers: No.

### Inclusion Criteria:

Participant must have histologically or cytologically confirmed non-small cell lung cancer (NSCLC) that is metastatic or unresectable. Participants must have either progressed after prior standard of care therapy (Cohorts C and hepatocyte growth factor receptor gene [MET]-1: epidermal growth factor receptor [EGFR] tyrosine kinase inhibitor [TKI]; Cohort D: platinum-based chemotherapy; MET-2: per regional standard of care; Cohorts wild-type adenocarcinoma (WT-Ad) and wild-type squamous cell carcinoma (WT-Sq): platinum-containing chemotherapy and programmed death-1/ligand-1 (PD-1/L1) therapy, either as a combined regimen or as separate lines of therapy) for metastatic disease, or be ineligible for, or have refused all other currently available therapeutic options. In cases where participants refuse currently available therapeutic options, this must be documented in the study records. For Part 1 Chemotherapy Combination Cohort only: Participants must have histologically or cytologically confirmed NSCLC that is metastatic or unresectable and be eligible for treatment with combination carboplatin and pemetrexed, in accordance with standard of care, and be willing to receive additional investigational therapy with Amivantamab.
For Part 1 Combination Dose Escalation with lazertinib only: Participants must have been diagnosed with EGFR Exon 19del or exon 21 L858R activating mutation and (a) be treatment naïve for metastatic disease, without access to third generation TKI in the front-line setting, or (b) have progressed after front-line treatment with first (erlotinib or gefitinib) or second generation (afatinib) TKI and are ineligible for Cohort MET-1, or (c) have been treated with a third generation TKI (e.g., osimertinib) in either the front line or second-line setting, and are not eligible for enrollment in either Cohort C or MET-1. For Part 1 Chemotherapy Combination Cohort: Participants may be diagnosed with EGFR mutated or EGFR wild type NSCLC. For Part 2 Cohorts C, D, MET-1, and MET-2 only: Participants must also have disease with a previously diagnosed activating epidermal growth factor receptor (EGFR) mutation (includes both inhibitor sensitive primary mutations such as Exon 19 deletion and exon 21 L858R (Cohort C, E, and MET-1), as well as marketed TKI-resistant mutations such as Exon 20 insertion (Cohort C, D and MET-1) or activating cMet Exon 14 skipping mutation (Cohort MET-2). Documentation of primary activating EGFR or cMet mutation eligibility by CLIA-certified laboratory (or equivalent) testing is required. For Part 2 Cohorts WT-Ad and WT-Sq: Participants must have wild-type EGFR, anaplastic lymphoma kinase (ALK), and absence of MET Exon 14 skipping mutation as tested by the Food and Drug Administration (FDA) approved test or a CLIA-certified laboratory (or equivalent). The pathology report or equivalent must be in the medical record for verification. Where testing for EGFR and ALK are not part of standard of care for participants with squamous cell carcinoma histology, documentation of the absence of these mutations is not necessary for enrollment into the WT-Sq cohort.
For Part 1: Participant must have evaluable disease. For Part 2: Participant must have measurable disease according to Response Criteria in Solid Tumors (RECIST) v1.1.
For Part 2: Cohorts A and B: Participants EGFR mutated disease must have most recently progressed following treatment with a marketed EGFR inhibitor. Exception: In participants diagnosed with mutations associated with de novo EGFR inhibitor resistance (for example, Exon 20 insertions), only previous treatment with combination platinum-based chemotherapy is required. Cohort C: Participants with primary EGFR mutated disease, with a documented EGFR alteration (example, C797S) mediating resistance to previous treatment with a third generation EGFR TKI (for example, osimertinib), in participants with primary Exon 20ins disease, the documented EGFR alteration may arise following treatment with a TKI with known activity against Exon 20ins disease (for example, poziotinib). Cohort D: participants must have been previously diagnosed with an EGFR Exon 20 insertion and have not been previously treated with a TKI with known activity against Exon 20ins disease (example, poziotinib). Cohort MET-1: Participants with documented primary EGFR mutated disease and documented MET amplification or MET mutation after progression on any EGFR TKI. Participants with disease characterized by both MET amplification and EGFR resistance mutations to prior third generation EGFR TKI will be preferentially enrolled into Cohort C. Participants may have received or have been intolerant to prior platinum-based chemotherapy. Cohort MET-2: Participants with documented primary MET Exon 14 skipping mutation non-small cell lung cancer (NSCLC). Cohort E (combination Amivantamab and lazertinib): Participants must have been diagnosed with EGFR exon 19del or exon 21 L858R activating mutation and have progressed after first or second-line treatment with a third generation TKI (e.g., osimertinib). Cohort WT-Ad: Participant must have been diagnosed with NSCLC of adenocarcinoma histology, with positive EGFR and/or MET expression as detected on a validated immunohistochemistry (IHC) assay performed by the central laboratory and have progressed on prior platinum containing chemotherapy and PD-1/L1 therapy, either as a combined regimen or as a separate line of therapy. Eligibility may be determined through IHC analysis of either archival (pre-screening) or mandatory fresh tumor tissue collected during the Screening period. Cohort WT-Sq: Participant must have been diagnosed with NSCLC of squamous cell carcinoma histology, with positive EGFR and/or MET expression as detected on a validated IHC assay performed by the central laboratory and have progressed on prior platinum-containing chemotherapy and PD-1/L1 therapy, either as a combined regimen or as a separate line of therapy. Eligibility may be determined through IHC analysis of either archival (pre-screening) or mandatory fresh tumor tissue collected during the Screening.
Participant must have Eastern Cooperative Oncology Group (ECOG) performance status 0 or 1.

### Exclusion Criteria:

Participant has uncontrolled inter-current illness, including but not limited to poorly controlled hypertension, or diabetes, ongoing or active infection, (that is, has discontinued all antibiotics for at least one week prior to first dose of study drug), or psychiatric illness/social situation that would limit compliance with study requirements. Participants with medical conditions requiring chronic continuous oxygen therapy are excluded. For Part 1 Chemotherapy Combination Cohort only: additionally, participants with active bleeding diathesis.
Participant has had prior chemotherapy, targeted cancer therapy, immunotherapy, or treatment with an investigational anticancer agent within 2 weeks or 4 half-lives whichever is longer, before the first administration of study drug. For agents with long half-lives, the maximum required time since last dose is 4 weeks. Toxicities from previous anti-cancer therapies should have resolved to baseline levels or to Grade 1 or less, (except for alopecia [any grade], Grade less than or equal to [<=] 2 peripheral neuropathy, and Grade less than [<] 2 hypothyroidism stable on hormone replacement). For Part 1 Combination Dose Escalation: Any previous treatment with systemic anti-cancer immunotherapy, including but not limited to anti-PD-1, anti-PD-L1, and anti-CTLA-4 agents. For Part 1 Chemotherapy Combination Cohort only: Any previous treatment with systemic anti-cancer immunotherapy in the past 3 months or localized radiotherapy to lung within the past 6 months. For Part 2 only: Cohorts A and B: Prior treatment with chemotherapy for metastatic disease is not allowed unless the tumor mutation carries de-novo resistance to EGFR TKI (example, Exon 20 insertions). Cohort C and MET-1: Prior treatment with more than 2 lines of cytotoxic chemotherapy for metastatic disease (maintenance therapy is not included). Cohort D: Previous treatment with an EGFR TKI with activity against EGFR Exon 20 insertions (such as poziotinib). Cohort E (combination Amivantamab and lazertinib): Any previous treatment in the metastatic setting with other than a first, second, or third generation EGFR TKI. Cohorts WT-Ad and WT-Sq: more than three lines of prior systemic therapy in the metastatic setting.
Participants with untreated brain metastases. Participants with definitively, locally-treated metastases that are clinically stable and asymptomatic for at least 2 weeks and who are off or receiving low-dose corticosteroid treatment (<=10 mg prednisone or equivalent) for at least 2 weeks prior to study treatment are eligible. Exception: participants with asymptomatic, untreated brain metastases, each less than 1 cm in diameter, may be eligible for Amivantamab and lazertinib combination therapy in the Part 1 Combination Dose Escalation or Part 2 Combination Expansion Cohort E.
Participant has a history of malignancy other than the disease under study within 3 years before Screening (exceptions are squamous and basal cell carcinomas of the skin and carcinoma in situ of the cervix, or malignancy that in the opinion of the investigator, with concurrence with the sponsor's medical monitor, is considered cured with or minimal risk of recurrence within a year from Screening).
Participant has not fully recovered from major surgery or significant traumatic injury prior the first dose of study drug or expects to have major surgery during the study period or within 6 months after the last dose of study drug.
Participant has, or will have, any of the following: a. An invasive operative procedure with entry into a body cavity, within 4 weeks or without complete recovery before Cycle 1 Day 1. Thoracentesis, if needed, and percutaneous biopsy for baseline tumor tissue sample may be done less than 4 weeks prior to Cycle 1 Day 1, as long as the participant has adequately recovered from the procedure prior to the first dose of study drug in the clinical judgement of the investigator; b. Significant traumatic injury within 3 weeks before the start of Cycle 1 Day 1 (all wounds must be fully healed prior to Day 1); c. Any medical condition that requires intact wound healing capacity and is expected to endanger subject safety if wound healing capacity would be severely reduced during administration of the investigational agent; d. Expected major surgery while the investigational agent is being administered or within 6 months after the last dose of study drug.

### Results for Treatment-Naïve Cohort (N=20)

CHRYSALIS (NCT02609776) evaluated the combination of amivantamab (ami) and lazertinib (laz) in treatment-naïve patients (pts) with epidermal growth factor receptor (EGFR)-mutated NSCLC. As previously reported, 20 pts achieved a partial response (overall response rate of 100%) but interpretation of long-term outcomes was limited by the length of follow up (Cho Ann Oncol 2020; 31: suppl_4, 12580; Cho J Thorac Oncol 2022; 17:S126, P1.16-01). Herein, long-term results are presented from this treatment-naïve cohort.

The treatment-naive cohort enrolled pts with EGFR exon 19 deletion (ex19del) or exon 21 L858R mutated advanced NSCLC. All pts received 1050 mg IV ami (1400 mg if ≥80 kg) and 240 mg oral laz. Response was assessed by the investigator per RECIST v1.1. Circulating tumor DNA (ctDNA) was analyzed from plasma samples prior to initiation of treatment, at Cycle 3 Day 1, and at end of treatment (EOT).

Of the 20 pts enrolled in the treatment-naive cohort (median 62.5 years, 55% women, all Asian), 11 had EGFR ex19del and 9 had exon 21 L858R NSCLC. The median follow-up and duration of treatment were 33.6 and 33.5 months, respectively. Ten (50%) pts were progression-free and remained on treatment, including 7 of 11 (64%) with ex19del and 3 of 9 (33%) with exon 21 L858R. The median duration of response (DOR), median progression-free survival (PFS), and median overall survival (OS) were not estimable. The estimated landmark PFS rate was 85% at 12 months, 65% at 24 months, and 51% at 36 months. Of note, 2 (10%) pts were treated beyond progression. The longest ongoing pt had a duration of treatment of 37.2 months and DOR of 35.7 months. Treatment-related dose interruptions, reductions, and discontinuations of either ami and laz occurred in 7 (35%) pts, 8 (40%) pts, and 1 (5%) pt, respectively. The safety profile was consistent with prior reports, with predominantly on-target EGFR- or MET-related adverse events.

Among the 10 pts who discontinued treatment, 4 submitted samples for ctDNA analysis at both baseline and EOT. There was 1 pt with new PIK3CA mutations, 1 with low-level HER2 amplification, 1 pt with a new CCNE1 and EGFR amplification, and 1 pt with no new mutations detected. Updated data on ctDNA at EOT may be available at the time of congress presentation.

At a median duration of treatment of 33.5 months, median DOR, PFS, and OS have not been reached in treatment-naïve pts receiving ami+laz, with 50% remaining progression-free and on treatment. No new safety signals were identified.

### Example 2. MARIPOSA Clinical Study

MARIPOSA (NCT04487080) is an international Phase 3 randomized study of amivantamab and lazertinib combination therapy versus osimertinib versus lazertinib as first-line treatment in approximately 1000 subjects with EGFR-mutated locally advanced or metastatic NSCLC (Study 73841937NSC3003, also known as NSC3003, and Mariposa).

The study includes a screening phase, a Treatment Phase, and a Follow-up Phase. Participants must complete screening procedures within 28 days before randomization. To be randomized, all participants must have been previously diagnosed with NSCLC, characterized by exon 19del or exon 21 L858R substitution EGFR mutations.

The Treatment Phase for a participant will begin on Cycle 1 Day 1 and continue as 28-day cycles until the End of Treatment visit, approximately 30 days after discontinuation of study treatment. Participants who discontinue study treatment for any reason will be followed for survival and symptomatic progression in the Follow-up Phase. The Follow-up Phase starts after the End of Treatment visit and continues until the end of study, death, lost to follow-up, or withdrawal of consent, whichever comes first.

### DESCRIPTION

The purpose of this study is to assess the efficacy of the amivantamab and lazertinib combination, compared with osimertinib, in participants with epidermal growth factor receptor (EGFR) mutation (exon 19 deletions [Exon 19del] or exon 21 L858R substitution) positive, locally advanced or metastatic non-small cell lung cancer (NSCLC).

Worldwide, lung cancer is the most commonly diagnosed cancer. In NSCLC the most prevalent actionable driver mutations result in the activation of epidermal growth factor receptor (EGFR). Osimertinib and Lazertinib are EGFR tyrosine kinase inhibitors (TKIs). Amivantamab is a novel bispecific antibody that targets the extracellular domain of both EGFR and MET and can inhibit tumor growth driven by EGFR and mesenchymal-epithelial transition (MET) receptors. Lazertinib inhibits primary activating exon 19del and exon 21 L858R substitution EGFR mutations, and the EGFR T790M+ resistance mutation. The hypothesis is that the amivantamab and lazertinib combination (Arm A) will demonstrate superior PFS compared with single-agent osimertinib (Arm B). The study consists of 3 phases: Screening Phase, Treatment Phase and Follow-up Phase. Participants will undergo response evaluation criteria in solid tumors (RECIST 1.1), pharmacokinetics, and safety evaluations (adverse events, laboratory tests, vital sign measurements, physical examinations).

### Study Design

Study Type: Interventional (Clinical Trial).
Estimated Enrollment: 1074 participants.
Allocation: Randomized.
Intervention Model: Parallel Assignment.
Masking: Triple (Participant, Investigator, Outcomes Assessor).
Masking Description: Only Arm B and C will be masked to all (Double-blind).
Primary Purpose: Treatment.
Official Title: A Phase 3, Randomized Study of Amivantamab and Lazertinib Combination Therapy Versus Osimertinib Versus Lazertinib as First-Line Treatment in Patients With EGFR-Mutated Locally Advanced or Metastatic Non-Small Cell Lung Cancer.

### Arms and Interventions

TABLE 2

| Arm | Intervention/treatment |
|---|---|
| Experimental: Treatment Arm A (Open- | Drug: Amivantamab |
| label): Amivantamab and Lazertinib | Participants will receive amivantamab |
| Participants will receive amivantamab 1050 | intravenously. |
| milligram (mg) intravenously (IV) for body | Other Name: JNJ-61186372 |
| weight less than (<) 80 kilogram (kg) and | Drug: Lazertinib |
| 1400 mg for body weight greater than or | Participants will receive lazertinib tablets |
| equal to (>=) 80 kg in 28-day cycles: once | orally. |
| weekly in Cycle 1 (with a split dose on | Other Name: JNJ-73841937 and YH-25448 |
| Days 1-2), and then every 2 weeks in | |
| subsequent cycles. Lazertinib will be | |
| administered 240 mg (80*3) orally once | |
| daily. | |
| Active Comparator: Treatment Arm B | Drug: Osimertinib |
| (Double-blind): Osimertinib + Placebo | Participants will receive osimertinib |
| Lazertinib | capsules orally. |
| Participants will receive osimertinib 80 mg | Drug: Placebo |
| orally once daily plus matching placebo of | Participants will receive matching placebo |
| lazertinib 240 mg (80*3) orally once daily. | orally. |
| Experimental: Treatment Arm C (Double- | Drug: Lazertinib |
| blind): Lazertinib + Placebo Osimertinib | Participants will receive lazertinib tablets |
| Participants will receive lazertinib 240 mg | orally. |
| (80*3) orally once daily plus matching | Other Name: JNJ-73841937 and YH-25448 |
| placebo of osimertinib 80 mg orally once | Drug: Placebo |
| daily. | Participants will receive matching placebo |
| | orally. |

### Outcome Measures

### Primary Outcome Measures

1. Progression-Free Survival (PFS) According to RECIST v1.1 by Blinded Independent Central Review (BICR) [Time Frame: Up to approximately 42 months]. PFS is defined as the time from randomization until the date of objective disease progression or death, whichever occurred first, based on BICR using response evaluation criteria in solid tumors (RECIST) v1.1.

### Secondary Outcome Measures

1. Overall Survival (OS) [Time Frame: Up to approximately 60 months (time from the date of randomization until the date of death due to any cause)]. Overall Survival is defined as the time from the date of randomization to the date of participant's death due to any cause.
2. Objective Response Rate (ORR) [Time Frame: Up to approximately 42 months]. ORR is defined as the percentage of participants who achieve either a complete response (CR) or partial response (PR) as defined by BICR using RECIST v1.1 criteria.
3. Duration of Response (DOR) [Time Frame: Up to approximately 42 months]. DOR is defined as the time from the date of first documented response (CR or PR) until the date of documented progression or death, whichever comes first, only for participants who achieve CR or PR as determined by the investigator using RECIST v1.1 criteria.
4. Progression-Free Survival After First Subsequent Therapy (PFS2) [Time Frame: Up to approximately 42 months]. The PFS2 is defined as the time from randomization until the date of second objective disease progression, after initiation of subsequent anticancer therapy, based on investigator assessment (after that used for PFS) or death, whichever comes first.
5. Time to Symptomatic Progression (TTSP) [Time Frame: Up to approximately 42 months]. TTSP is defined as the time from randomization to documentation in the electronic case report form (eCRF) of any of the following (whichever occurs earlier): onset of new symptoms or symptom worsening that is considered by the investigator to be related to lung cancer and requires either a change in anticancer treatment and/or clinical intervention to manage symptoms.
6. Intracranial PFS [Time Frame: Up to approximately 42 months]. Intracranial PFS is defined as the time from randomization until the date of objective intracranial disease progression or death, whichever comes first, based on BICR using RECIST v1.1.
7. Incidence and Severity of Adverse Events (AEs) [Time Frame: Up to approximately 60 months]. Incidence and severity of treatment emergent adverse events (TEAEs) will be reported. Any adverse event occurring at or after the initial administration of study treatment through the day of last dose plus 30 days, or until the start of subsequent anticancer therapy (if earlier), is considered to be treatment emergent.
8. Number of Participants with Clinical Laboratory Abnormalities [Time Frame: Up to approximately 60 months]. Number of participants with clinical laboratory abnormalities (serum chemistry, hematology, blood coagulation, and urine samples) will be reported.
9. Number of Participants with Vital Signs Abnormalities [Time Frame: Up to approximately 60 months]. Number of participants with vital signs abnormalities (temperature, heart rate, respiratory rate, oxygen saturation, blood pressure) will be reported.
10. Number of Participants with Physical Examination Abnormalities [Time Frame: Up to approximately 60 months]. Number of participants with physical examination abnormalities will be reported.
11. Serum Concentration of Amivantamab [Time Frame: Up to approximately 42 months]. Serum samples will be analyzed to determine concentrations of amivantamab.
12. Plasma Concentration of Lazertinib [Time Frame: Up to approximately 42 months]. Plasma samples will be analyzed to determine concentrations of lazertinib.
13. Number of Participants with Anti-Amivantamab Antibodies [Time Frame: Up to approximately 42 months]. Number of participants with antibodies to amivantamab will be reported.
14. Change from Baseline in Non-Small Cell Lung Cancer-Symptom Assessment Questionnaire (NCSLC-SAQ) [Time Frame: Baseline Up to approximately 42 months]. The NSCLC-SAQ contains 7 items that assess cough, pain, dyspnea, fatigue, and poor appetite over a 7-day recall period. Each multi-item scale and individual item will be summarized using count and percent by visit.
15. Change from Baseline in European Organization of Research and Treatment of Cancer Quality of Life Questionnaire Core 30 (EORTC-QLQ-C30) [Time Frame: Baseline Up to approximately 42 months]. EORTC-QLQ-C30 is a core 30-item questionnaire for evaluating the health-related quality of life (HRQOL) of participants participating in cancer clinical studies.
16. Time to Subsequent Therapy (TTST) is defined as the time from the date of randomization in a clinical trial to the start date of the subsequent anticancer therapy following study treatment discontinuation or death, whichever comes first.

### Eligibility Criteria.

Ages Eligible for Study: 18 Years and older (Adult, Older Adult).
Sexes Eligible for Study: All.
Accepts Healthy Volunteers: No.

### Inclusion Criteria:

Participant must have newly diagnosed histologically or cytologically confirmed, locally advanced or metastatic non-small cell lung cancer (NSCLC) that is treatment naive and not amenable to curative therapy including surgical resection or chemoradiation.
The tumor harbors exon 19 deletions (exon 19del) or exon 21 L858R substitution, as detected by a food and drug administration (FDA)-approved or other validated test in a clinical laboratory improvement amendments (CLIA) certified laboratory (sites in the United states [US]) or an accredited local laboratory (sites outside of the US) in accordance with site standard of care.
Mandatory submission of unstained tissue from tumor (in a quantity sufficient to allow for central analysis of EGFR mutation status and blood (for circulating tumor deoxyribonucleic acid [ctDNA], digital droplet polymerase chain reaction [ddPCR], and pharmacogenomic analysis).
Any toxicities from prior anticancer therapy must have resolved to common terminology criteria for adverse events (CTCAE) Grade 1 or baseline level.
Participant must have at least 1 measurable lesion, according to response evaluation criteria in solid tumors (RECIST) v1.1 that has not been previously irradiated. Measurable lesions should not have been biopsied during screening, but if only 1 non-irradiated measurable lesion exists, it may undergo a diagnostic biopsy and be acceptable as a target lesion, provided the baseline tumor assessment scans are performed at least 14 days after the biopsy.

### Exclusion Criteria:

Participant has received any prior systemic treatment at any time for locally advanced Stage III or metastatic Stage IV disease (adjuvant or neoadjuvant therapy for Stage I or II disease is allowed, if administered more than 12 months prior to the development of locally advanced or metastatic disease).
Participant has an active or past medical history of leptomeningeal disease.
Participant with untreated spinal cord compression. A participant that has been definitively treated with surgery or radiation and has a stable neurological status for at least 2 weeks prior to randomization is eligible provided they are off corticosteroid treatment or receiving low-dose corticosteroid treatment less than or equal to (<=) 10 milligrams per day (mg/day) prednisone or equivalent.
Participant has an active or past medical history of interstitial lung disease (ILD)/pneumonitis, including drug-induced or radiation ILD/pneumonitis.
Participant has known allergy, hypersensitivity, or intolerance to the excipients used in formulation of amivantamab, lazertinib, or osimertinib, or any contraindication to the use of Osimertinib.
Participant has symptomatic brain metastases. A participant with asymptomatic or previously treated and stable brain metastases may participate in this study.
   Amivantamab Plus Lazertinib Vs Osimertinib as First-Line Treatment in Patients with EGFR-Mutated, Advanced Non-Small Cell Lung Cancer (NSCLC): Primary Results from MARIPOSA, a Phase 3, Global, Randomized, Controlled Trial

The pivotal Phase 3 MARIPOSA study met its primary endpoint with a statistically significant and clinically meaningful improvement in progression-free survival (PFS) in patients receiving RYBREVANT® plus lazertinib compared to osimertinib. The combination of RYBREVANT® and lazertinib demonstrated a safety profile consistent with previously reported data on the combination.

MARIPOSA (NCT04487080), which enrolled 1,074 patients, is a randomized, open-label Phase 3 study evaluating RYBREVANT® in combination with lazertinib versus osimertinib and versus lazertinib alone in the first-line treatment of patients with locally advanced or metastatic NSCLC with EGFR exon 19 deletions (ex19del) or substitution mutations such as exon 21 L858R. The primary endpoint of the study is PFS (using RECIST v1.1 guidelines) as assessed by blinded independent central review (BICR). Secondary endpoints include OS, objective response rate (ORR), duration of response (DoR), intracranial PFS, PFS after first subsequent therapy (PFS2), time to subsequent therapy (TTST), time to symptomatic progression (TTSP) and safety.

Methods: Patients with treatment-naïve, EGFR-mutated (Ex19del or exon 21 L858R) locally advanced or metastatic NSCLC were randomized 2:2:1 to amivantamab+lazertinib (open-label), osimertinib (blinded), or lazertinib (blinded).

Results: In total, 1074 patients were randomized (amivantamab+lazertinib, 429; osimertinib, 429; lazertinib, 216). Baseline characteristics were well balanced; median age was 63 years, 62% were female, 59% Asian, and 41% with history of brain metastases.

At a median follow-up of 22.0 months, the median PFS was 23.7 months (95% CI, 19.1-27.7) for amivantamab+lazertinib and 16.6 months (95% CI, 14.8-18.5) for osimertinib (HR, 0.70; 95% CI, 0.58-0.85; P<0.001).

PFS benefit of amivantamab+lazertinib was consistent across predefined subgroups. ORR was 86% (95% CI, 83-89) for amivantamab+lazertinib vs 85% (95% CI, 81-88) for osimertinib, with median DoR of 25.8 months (95% CI, 20.1-NE) vs 16.8 months (95% CI, 14.7-18.5), respectively, among confirmed responders. The median DoR was longer by 9 months in the amivantamab+lazertinib arm compared with the osimertinib arm or lazertinib arm.

At interim OS analysis, the median was not reached for either arm; however, a strong trend favored amivantamab+lazertinib vs osimertinib with an HR of 0.80 (95% CI, 0.61 to 1.05; P=0.1). Amivantamab+lazertinib was associated with a higher rate of Venous Thromboembolism (VTE); mostly grade 1-2, occurring early, and effectively managed with anticoagulation. Higher rates of EGFR- and MET-related adverse events were seen with amivantamab+lazertinib vs osimertinib.

Conclusions: Amivantamab+lazertinib provided statistically significant and clinically meaningful improvement in PFS vs osimertinib with a 30% reduction in the risk of progression or death compared with osimertinib, with a numerically higher DoR and a strong trend for improved OS. The safety profile of amivantamab+lazertinib was consistent with prior reports. MARIPOSA establishes amivantamab+lazertinib as a new first-line, standard of care for EGFR-mutated, advanced NSCLC.

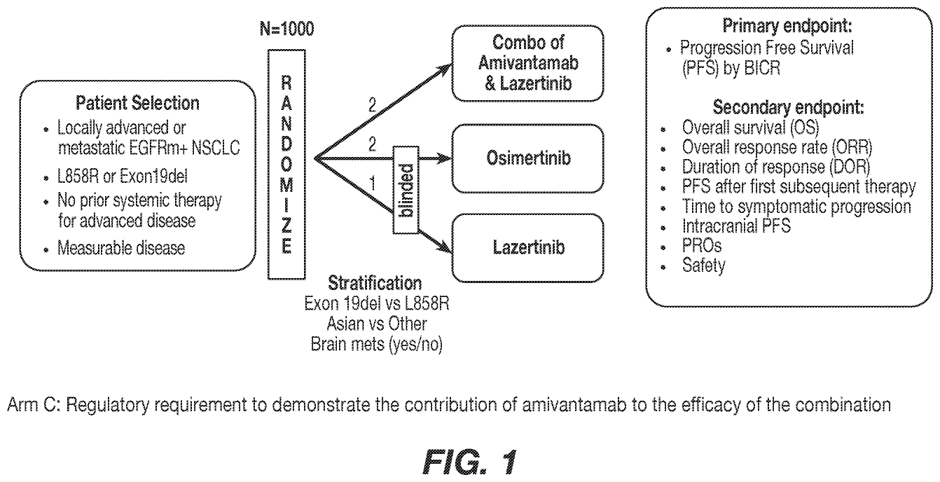

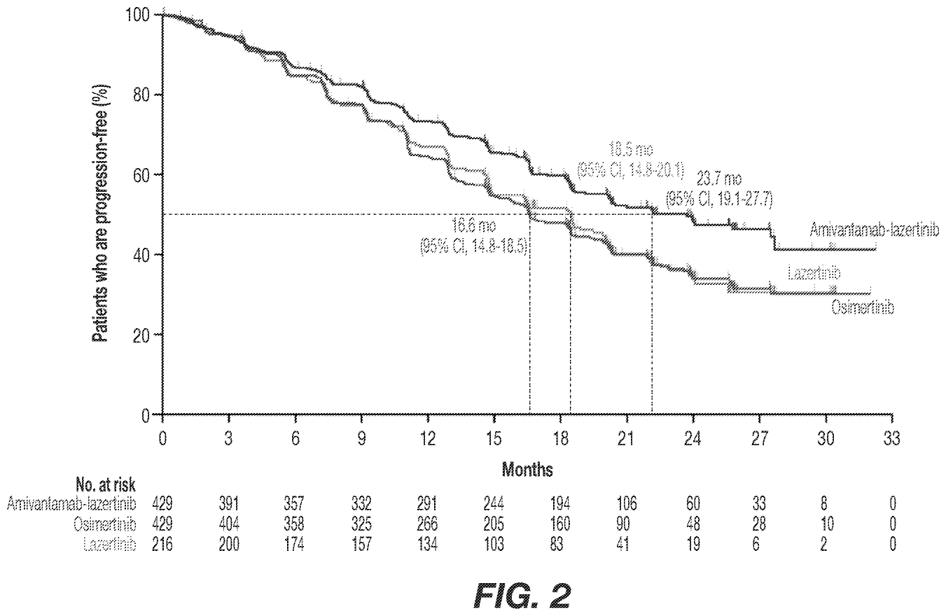

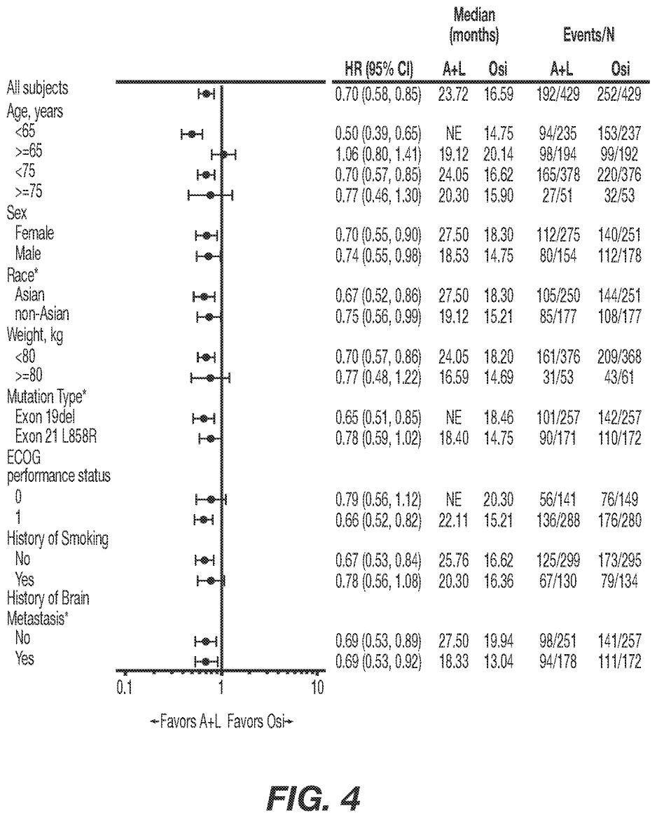

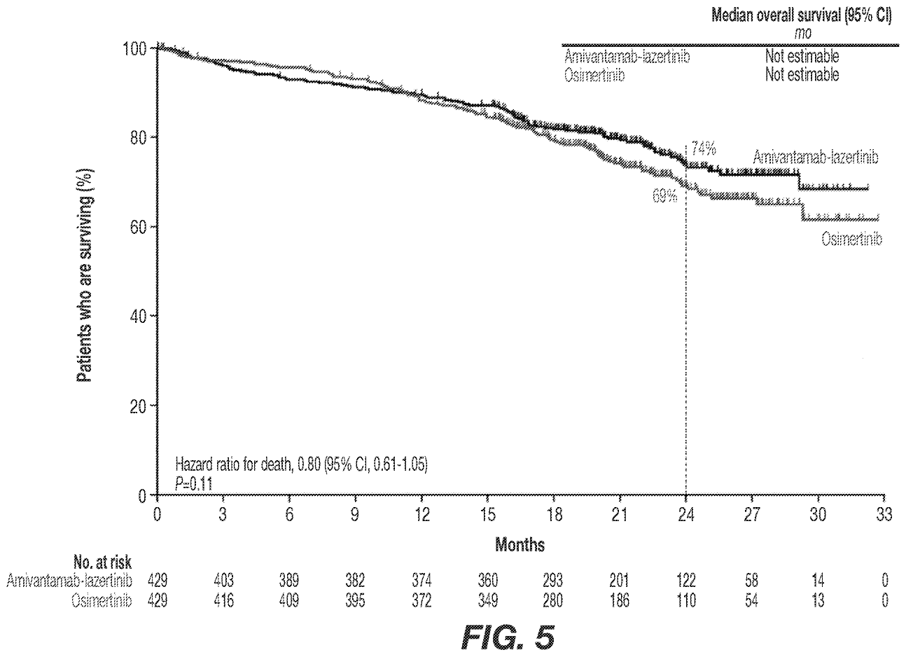

An exemplary schematic overview of the MARIPOSA clinical study is shown in FIG. 1. PFS results are shown in FIGS. 2-4. OS results are shown in FIG. 5.

TABLE 3
Summary of Progression-Free Survival (PFS) by BICR
TEFPFS01S1RC: Summary of Progression-free Survival-Primary Analysis-
Stratified Anaylsis-BICR: Full Analysis Set (Study 73841937NSC3003)

| | Amivantamab + | | |
|---|---|---|---|
| | Lazertinib | Osimertinib | Lazertinib |
| Analysis set: Full | 429 | 429 | 216 |
| Event | 192 (44.8%) | 252 (58.7%) | 121 (56.0%) |
| Censored | 237 (55.2%) | 177 (41.3%) | 95 (44.0%) |
| Time to event (months) | | | |
| 25th percentile (95% CI) | 11.07 [symbol] | 9.23 (7.49, 10.94) | 9.23 (7.43, 11.10) |
| Median (95% CI) | 23.72 (19.12, 27.66) | 16.59 (14.78, 18.46) | [symbol] |
| 75th percentile (95% CI) | NE (NE, NE) | NE (27.50, NE) | NE (24.05, NE) |
| Range | (0.0+, 32.3+) | (0.0+, 32.0+) | (0.0+, 30.3+) |
| 6-month event-free rate (95% CI) | 0.87 (0.83, 0.90) | 0.85 (0.83, 0.88) | 0.85 (0.79, 0.89) |
| 12-month event-free rate (95% CI) | 0.73 (0. [symbol] , 0.77) | 0.65 ( [symbol] ) | 0.67 (0.00, 0.73) |
| 18-month event-free rate (95% CI) | 0.00 (0.55, 0.64) | 0.48 (0.43, 0.53) | 0.52 (0.44, 0.58) |
| 24-month event-free rate (95% CI) | 0.48 (0.42, 0.54) | 0.34 (0.26, 0.39) | 0.35 (0.27, 0.42) |
| 30-month event-free rate (95% CI) | 0.43 (0.32, 0.50) | 0.30 (0.24, 0.36) | 0.30 (0.22, 0.39) |
| Amivantamab + Lazertinib vs Osimertinib | | | |
| p-value [symbol] | 0.0002 | | |
| Hazard ratio (95% CI) [symbol] | 0.70 (0.58, 0.85) | | |
| Amivantamab + Lazertinib vs Lazertinib | | | |
| p-value [symbol] | [symbol] | | |
| Hazard ratio (95% CI) [symbol] | 0.72 (0.57, 0.90) | | |

Key: [symbol]
= censored observation;
NE = not estimable [symbol]
p-value is from a log-rank test stratified by mutation type (Exon 19del or Exon 21 L858R), race (Asian or Non-Asian) and history of brain metastatis (present or absent). [symbol]
Hazard ratio is from a stratified proportional hazards model. Hazard ratio <1 favors Amivantamab + Lazertinib. [symbol]
indicates data missing or illegible when filed

TABLE 4
Summary of Overal Survival results
TEFOS01S: Summary of Overall Survival-
Stratified Anaylsis; Full Analysis Set (Study 73841937NSC3003)

| | Amivantamab + | | |
|---|---|---|---|
| | Lazertinib | Osimertinib | Lazertinib |
| Analysis set: Full | 429 | 429 | 216 |
| Event | 97 (22.6%) | 117 (27.3%) | 56 (25.9%) |
| Censored | 332 (77.4%) | 312 (72.7%) | 160 (74.1%) |
| Time to event (months) | | | |
| 25th percentile (95% CI) | 23.62 (21.03 NE) | 20.30 (18.00, 23.59) | 20.30 (14.92, NE) |
| Median (95% CI) | NE (NE, NE) | NE (NE, NE) | NE (NE, NE) |
| 75th percentile (95% CI) | NE (NE, NE) | NE (NE, NE) | NE (NE, NE) |
| Range | (0.0+, 32.3+) | (0.3, 32.7+) | (0.0+, 32.1+) |
| 6-month event-free rate (95% CI) | 0.93 (0.90, 0.95) | 0.96 (0.93, 0.97) | 0.95 (0.91, 0.87) |
| 12-month event-free rate (95% CI) | 0.90 (0.86, 0.92) | 0.88 (0.85, 0.91) | 0.86 (0.81, 0.90) |
| 18-month event-free rate (95% CI) | 0.82 (0.78, 0.85) | 0.79 (0.75, 0.83) | 0.78 (0.71, 0.83) |
| 24-month event-free rate (95% CI) | 0.74 (0.69, 0.78) | 0.60 (0.64, 0.74) | 0.71 (0.61, 0.78) |
| 30-month event-free rate (95% CI) | 0.68 (0.60, 0.76) | 0.62 (0.52, 0.70) | 0.68 (0.58, 0.75) |
| Amivantamab + Lazertinib vs Osimertinib | | | |
| p-value [symbol] | [symbol] | | |
| Hazard ratio (95% CI) [symbol] | 0.80 (0.01, 1.05) | | |
| Amivantamab + Lazertinib vs Lazertinib | | | |
| p-value [symbol] | 0.2343 | | |
| Hazard ratio (95% CI) [symbol] | 0.82 (0.59, 1.14) | | |

Key: [symbol]
= [symbol] observation;
NE = not estimable [symbol]
p-value is from a log-rank test stratified by mutation type (Exon 19del or Exon 21 L858R), race (Asian or Non-Asian) and history of brain metastatis (present or absent). [symbol]
Hazard ratio is from a stratified proportional hazards model. Hazard ratio <1 favors Amivantamab + Lazertinib. [symbol]
indicates data missing or illegible when filed

TABLE 5
Summary of Objective Response by BICR
TEFORR01lrc: Summary of Objective Response Rate Based on RECIST v1.1
Criteria in Subjects With Measurable Disease at Baseline-BICR:
Full Analysis Set (Study 73841937NSC3003)

| | Amivantamab + | | |
|---|---|---|---|
| | Lazertinib | Osimertinib | Lazertinib |
| Analysis set: Full | 429 | 429 | 216 |
| Number of subjects with measurable | 421 | 414 | 214 |
| disease at baseline | | | |
| Objective response rate (CR + PR) | 363 (86.2%) | 350 (84.5%) | 177 (82.7%) |
| 95% CI | (82.6%, 89.4%) | (80.7%, 87.9%) | (77.0%, 87.5%) |
| Amivantamab + Lazertinib vs Osimertinib | | | |
| p-value [symbol] | 0.4714 | | |
| Odds ratio (95% CI) [symbol] | 1.15 (0.78, 1.70) | | |
| Amivantamab + Lazertinib vs Lazertinib | | | |
| p-value [symbol] | 0.2400 | | |
| Odds ratio (95% CI) [symbol] | 1. [symbol] (0.83, 2.06) | | |
| Best Overall Response | | | |
| Complete Response (CR) | 29 (0.9%) | 15 (3.0%) | 9 (4.2%) |
| Partial Response (PR) | 334 (79.3%) | 335 (80.9%) | 168 (78.5%) |
| Stable Disease (SD) | 30 (7.1%) | 42 (10.1%) | 23 (10.7%) |
| Progressive Disease (PD) | 7 (1.7%) | 11 (2.7%) | 9 (4.2%) |
| Not Eval [symbol] (NE) | 21 (5.0%) | 11 (2.7%) | 5 (2.3%) |

Key:
CI = confidence interval
ap-value and odds ratio are from logistic regression model stratified by mutation type (Exon 19del or Exon 21 L858R), race (Asian or Non-Asian) and history of brain metastatis (present or absent). [symbol]
Hazard ratio is from a stratified proportional hazards model. Hazard ratio <1 favors Amivantamab + Lazertinib. [symbol]
indicates data missing or illegible when filed

TABLE 6
Summary of Confirmed DOR by BICR results
TEFDORC01lrc: Summary of Duration of Response in Confirmed Responders With
Measurable Disease at Baseline-BICR: Full Analysis Set (Study 73841937NSC3003)

| | Amivantamab + | | |
|---|---|---|---|
| | Lazertinib | Osimertinib | Lazertinib |
| Analysis set: Full | 429 | 429 | 216 |
| Number of subjects with measurable | 421 | 414 | 214 |
| disease at baseline | | | |
| Confirmed Responders (Confirmed | 336 | 314 | 160 |
| CR + Confirmed PR) | | | |
| Event | 127 ( [symbol] ) | 163 (51.9%) | 83 (51.9%) |
| Censored | 209 (62.2%) | 151 (48.1%) | 77 (48.1%) |
| Time to event (months) [symbol] | | | |
| 25th percentile (95% CI) | [symbol] | 9.26 (9.13, 10.25) | 9.23 (7.39, 11.10) |
| Median (95% CI) | 25.76 (20.14, NE) | 16.76 (14.75, 18.53) | 16.56 (14.75, 20.21) |
| 75th percentile (95% CI) | NE (NE, NE) | NE (NE, NE) | NE (NE, NE) |
| Range | (1.4, 28.5+) | (1.9+, 28.7+) | (1.9+, 28.6+) |
| Duration of response >=6 months | 290 (86.3%) | 267 (65.0%) | 132 (82.5%) |
| Duration of response >=12 months | 228 (67.9%) | 181 (57.6%) | 94 (58.8%) |
| Duration of response >=18 months | 115 (34.2%) | 86 (27.4%) | 40 (25.0%) |
| Duration of response >=24 months | 34 (10.1%) | 21 (6.7%) | 5 (3.1%) |

Key:
CI = confidence interval [symbol]
= censored observation;
NE = not estimable
Quartiles and 95% CIs are estimated with Kaplan-Meier method.
Note:
Percentages are based on the number of subjects who achieved Confirmed CR and Confirmed PR. [symbol]
indicates data missing or illegible when filed

TABLE 7
Summary of PFS2 results
TEFORR01lrc: Summary of Objective Response Rate Based on RECIST v1.1 Criteria in Subjects
With Measurable Disease at Baseline-BICR: Full Analysis Set (Study 73841937NSC3003)

| | Amivantamab + | | |
|---|---|---|---|
| | Lazertinib | Osimertinib | Lazertinib |
| Analysis set: Full | 429 | 429 | 216 |
| Event | 101 (23.5%) | 130 (30.3%) | 58 (26.9%) |
| Censored | 328 (76.5%) | 299 (60.7%) | 158 (73.1%) |
| Time to event (months) | | | |
| 25th percentile (95% CI) | 22.60 (18.00, 29.14) | 18.40 (15.93, 20.50) | 19.58 (14.62, 24.80) |
| Median (95% CI) | 30.42 (30.42, NE) | NE (29.31, NE) | 28.94E (28.94, NE) |
| 75th percentile (95% CI) | NE (30.42, NE) | NE (NE, NE) | NE (28.94, NE) |
| Range | (0.0+, 32.3+) | (0.3, 32.0+) | (0.0+, 30.3+) |
| 6-month event-free rate (95% CI) | 0.93 (0.90, 0.95) | 0.95 (0.93, 0.97) | 0.95 (0.91, 0.97) |
| 12-month event-free rate (95% CI) | 0.88 (0.85, 0.91) | 0.87 (1.83, 0.90) | 0.85 (0.79, 0.89) |
| 18-month event-free rate (95% CI) | 0.79 (0.75, 0.83) | 0.76 (0.71, 0.80) | 0.77 (0.71, 0.82) |
| 24-month event-free rate (95% CI) | 0.72 (0.67, 0.77) | 0.64 (0. [symbol] , 0.69) | 0.68 (0. [symbol] , 0.75) |
| 30-month event-free rate (95% CI) | 0.66 (0.55, 0.75) | 0.56 (0.46, 0.66) | 0.48 (0. [symbol] , 0.72) |
| Amivantamab + Lazertinib vs Osimertinib | | | |
| p-value [symbol] | 0.0314 | | |
| Hazard ratio (95% CI) [symbol] | 0.75 (0. [symbol] , 0.98) | | |
| Amivantamab + Lazertinib vs Lazertinib | | | |
| p-value [symbol] | 0.2925 | | |
| Hazard ratio (95% CI) [symbol] | 0.81 (0. [symbol] , 1.12) | | |

Key: [symbol]
= censored observation;
NE = not estimable [symbol]
p-value is from a log-rank test stratified by mutation type (Exon 19del or Exon 21 L858R), race (Asian or Non-Asian) and history of brain metastatis (present or absent). [symbol]
Hazard ratio is from a stratified proportional hazards model. Hazard ratio <1 favors Amivantamab + Lazertinib. [symbol]
indicates data missing or illegible when filed

TABLE 8
Summary of Time to Symptomatic Progression (TTSP) results
TEFTTSP015: Summary of Time to Symptomatic Progression-
Stratified Analysis; Full Analysis Set (Study 73841937NSC3003)

| | Amivantamab + | | |
|---|---|---|---|
| | Lazertinib | Osimertinib | Lazertinib |
| Anaylsis set: Full | 429 | 429 | 216 |
| Event | 125 (29.1%) | 167 (38.9%) | 70 (22.4%) |
| Symptomatic PD | 60 (16.1%) | 118 (27.5%) | 46 (21. [symbol] %) |
| Death without Symptomatic PD | 56 (13.1%) | 49 (11.4%) | 24 (11.1%) |
| Censored | 304 (70.0%) | 202 (61.1%) | 146 ( [symbol] %) |
| Time to event (months) | | | |
| 25th percentile (95% CI) | 37.58 (14.23, [symbol] ) | [symbol] (11.60, 15.90) | 13.80 (10.41, 18.56) |
| Median (95% CI) | NE (NE, NE) | 29.31 (29.31, NE) | NE (NE, NE) |
| 75th percentile (95% CI) | NE (NE, NE) | NE (NE, NE) | NE (NE, NE) |
| Range | (0.0+, 32.3+) | (0.0+, 32.7+) | (0.0+, 32.1+) |
| 6-month event-free rate (95% CI) | 0.88 (0.85, 0.91) | 0.90 (0.87, 0.93) | 0.90 (0.85, 0.93) |
| 12-month event-free rate (95% CI) | 0.82 (0.77, 0.85) | 0.79 (0.74, 0.82) | 0.79 (0.72, 0.84) |
| 18-month event-free rate (95% CI) | 0.74 (0.70, 0.78) | 0.67 (0.63, 0.72) | 0.71 (0.64, 0.76) |
| 24-month event-free rate (95% CI) | 0.67 (0.62, 0.72) | 0.59 (0.53, 0.64) | 0.65 (0.58, 0.72) |
| 30-month event-free rate (95% CI) | 0.64 (0.56, 0.71) | 0.49 (0.40, 0.58) | 0.61 (0.52, 0.69) |
| Amivantamub + Lazertinib vs Osimertinib | | | |
| p-value [symbol] | [symbol] | | |
| Hazard ratio (95% CI) [symbol] | 0.72 (0.57, 0.01) | | |
| Amivantamub + Lazertinib vs Lazertinib | | | |
| p-value [symbol] | 0.3063 | | |
| Hazard ratio (95% CI) [symbol] | 0.86 (0.64, 1.15) | | |

Key: [symbol]
= censored observation;
NE = not estimable [symbol]
p-value is from a log-rank test stratified by mutation type (Exon 19del or Exon 21 L858R), race (Asian or Non-Asian), and history of brain metastatis (present or absent). [symbol]
Hazard ratio is from a stratified proportional hazards model. Hazard ratio <1 favors Amivantanub + Lazertinib. [symbol]
indicates data missing or illegible when filed

TABLE 9
Summary of Time to Subsequent Therapy (TTST) results
TEFTTSP015: Summary of Time to Subsequent Therapy-Stratified Analysis;
Full Analysis Set (Study 73841937NSC3003)

| | Amivantamab + | | |
|---|---|---|---|
| | Lazertinib | Osimertinib | Lazertinib |
| Anaylsis set: Full | 429 | 429 | 216 |
| Event | 166 (38.7%) | 197 (45.9%) | 99 (45. [symbol] %) |
| Censored | 263 (61.3%) | 232 (54.1%) | 117 (54.2%) |
| Time to event (months) | | | |
| 25th percentile (95% CI) | 14.29 (11.40, 16.59) | 13.11 (10.58, 14.36) | 11.27 (8.80, 13.67) |
| Median (95% CI) | NE (26.78, NE) | 24.08 (22.01, 28.98) | 24.02 (19.45, NE) |
| 75th percentile (95% CI) | NE (NE , NE) | NE (NE, NE) | NE (NE , NE) |
| Range | (0.0+, 31.5+) | (0.3, 32.7+) | (0.0+, 32.1+) |
| 6-month event-free rate (95% CI) | 0.88 (0.85, 0.91) | 0.91 (0.88, 0.93) | 0.90 (0.85, 0.93) |
| 12-month event-free rate (95% CI) | 0.78 (0.74, 0.82) | 0.77 (0.73, 0.81) | 0.71 (0.66, 0.78) |
| 18-month event-free rate (95% CI) | 0.67 (0.63, 0.72) | 0.63 (0.58, 0.67) | 0.60 (0.53, 0.66) |
| 24-month event-free rate (95% CI) | 0.57 (0.52, 0.62) | 0.51 (0. [symbol] , 0. [symbol] ) | 0.50 (0.43, 0.58) |
| 30-month event-free rate (95% CI) | 0.53 (0.47, 0.59) | 0.42 (0. [symbol] , 0. [symbol] ) | 0.46 (0. [symbol] , 0.5 [symbol] ) |
| Amivantamub + Lazertinib vs Osimertinib | | | |
| p-value [symbol] | 0.0533 | | |
| Hazard ratio (95% CI) [symbol] | 0.82 (0. [symbol] , 1.00) | | |
| Amivantamub + Lazertinib vs Lazertinib | | | |
| p-value [symbol] | 0.04 [symbol] | | |
| Hazard ratio (95% CI) [symbol] | 0.78 (0.61, 1.00) | | |

Key: [symbol]
= censored observation;
NE = not estimable [symbol]
p-value is from a log-rank test stratified by mutation type (Exon 19del or Exon 21 L858R), race (Asian or Non-Asian), and history of brain metastatis (present or absent). [symbol]
Hazard ratio is from a stratified proportional hazards model. Hazard ratio <1 favors Amivantanub + Lazertinib. [symbol]
indicates data missing or illegible when filed

TABLE 10
Summary of intracranial PFS results in participants with baseline
brain metastasis
TEFICP [symbol] : Summary of Intracranial Progression-free Survival in Subjects With History of Brain
Metastatis-Stratified Analysis-BICR: Full Analysis Set (Study 73841937NSC3003)

| | Amivantamab + | | |
|---|---|---|---|
| | Lazertinib | Osimertinib | Lazertinib |
| Anaylsis set: Full | 429 | 429 | 216 |
| Number of subjects with history of | 178 | 172 | 86 |
| brain metastatis | | | |
| Event | 75 (42.1%) | 75 (43.6%) | 37 (43.0%) |
| Censored | 103 (37.9%) | 97 ( [symbol] %) | 49 ( [symbol] %) |
| Time to event (months) | | | |
| 25th percentile (95% CI) | [symbol] | [symbol] | [symbol] |
| Median (95% CI) | NE (38.43, NE) | 23.09 ( [symbol] , NE) | 2030 ( [symbol] , NE) |
| 75th percentile (95% CI) | NE (NE, NE) | NE (NE, NE) | NE (22.67, NE) |
| Range | (0.0+, 30.3+) | (0.0+, [symbol] +) | (0.0+, 28.6+) |
| 6-month event-free rate (95% CI) | 0.87 (0.81, 0.91) | 0.88 (0.82, 0.92) | [symbol] |
| 12-month event-free rate (95% CI) | 0.71 (0.64, 0.77) | 0.75 ( [symbol] ) | [symbol] |
| 18-month event-free rate (95% CI) | 0.59 (0.51, 0.66) | 0.58 ( [symbol] ) | [symbol] |
| 24-month event-free rate (95% CI) | 0.01 (0.42, 0. [symbol] ) | 0. [symbol] ( [symbol] ) | [symbol] |
| 30-month event-free rate (95% CI) | 0.31 (0.42, 0.59) | 0. [symbol] ( [symbol] ) | [symbol] |
| Amivantamub + Lazertinib vs Osimertinib | | | |
| p-value [symbol] | [symbol] | | |
| Hazard ratio (95% CI) [symbol] | [symbol] | | |
| Amivantamub + Lazertinib vs Lazertinib | | | |
| p-value [symbol] | [symbol] | | |
| Hazard ratio (95% CI) [symbol] | [symbol] | | |

Key: [symbol]
= censored observation;
NE = not estimable [symbol]
p-value is from a log-rank test stratified by mutation type (Exon 19del or Exon 21 L858R), race (Asian or Non-Asian). [symbol]
Hazard ratio is from a stratified proportional hazards model. Hazard ratio <1 favors Amivantanub + Lazertinib.
Note:
History of brain metastatis is based on investigator reported data [symbol] [symbol]
indicates data missing or illegible when filed

TABLE 11
Demographics and Baseline Disease Characteristics*

| | Amivantamab- | |
|---|---|---|
| | lazertinib | Osimertinib |
| Characteristic | (n = 429) | (n = 429) |
| Age | | |
| Median (range)-years | 64 (25-88) | 63 (28-88) |
| Category-no. (%) | | |
| <65 years | 235 (55) | 237 (55) |
| ≥65 to <75 years | 143 (33) | 139 (32) |
| ≥75 years | 51 (12) | 53 (12) |
| Sex-no. (%) | | |
| Female | 275 (64) | 251 (59) |
| Male | 154 (36) | 178 (41) |
| Race-no. (%) | | |
| Asian | 250 (58) | 251 (59) |
| White | 164 (38) | 165 (38) |
| American Indian or Alaska Native | 7 (2) | 7 (2) |
| Black or African American | 4 (1) | 3 (1) |
| Native Hawaiian or Pacific | 1 (0.2) | 1 (0.2) |
| Islander | | |
| Multiple | 1 (0.2) | 1 (0.2) |
| Unknown | 2 (0.5) | 1 (0.2) |
| Region of enrollment-no. (%) | | |
| North America | 19 (4) | 19 (4) |
| South America | 46 (11) | 33 (8) |
| Europe* | 96 (22) | 104 (24) |
| Asia† | 263 (61) | 266 (62) |
| Oceania | 5 (1) | 7 (2) |
| Body weight | | |
| Median (range)-kg | 62.5 (32-118) | 62.4 (35-109) |
| Category-no. (%) | | |
| <80 kg | 376 (88) | 368 (86) |
| ≥80 kg | 53 (12) | 61 (14) |
| Eastern Cooperative Oncology | | |
| Group performance status-no. | | |
| (%) | | |
| 0 | 141 (33) | 149 (35) |
| 1 | 288 (67) | 280 (65) |
| History or smoking-no. (%) | | |
| No | 299 (70) | 295 (69) |
| Yes | 130 (30) | 134 (31) |
| Time from initial diagnosis, median | 1.5 (0.2-207.9) | 1.4 (0.3-162.8) |
| (range)-months | | |
| Time from metastatic diagnosis, | 1.3 (0.2-24.1) | 1.2 (0.1-11.7) |
| median (range)-months | | |
| Histologic type-no. (%) | | |
| Adenocarcinoma | 417 (97) | 415 (97) |
| Large cell carcinoma | 3 (1) | 0 |
| Squamous cell carcinoma | 6 (1) | 5 (1) |
| Other§ | 2 (0.5) | 9 (2) |
| Not reported | 1 (0.2) | 0 |
| History of brain metastases-no. | 178 (41) | 172 (40) |
| (%) | | |
| Type of EGFR mutation-no. | | |
| (%)\|\| | | |
| Exon 19 deletion | 258 (60) | 257 (60) |
| Exon 21 L858R | 172 (40) | 172 (40) |

*ECOG denotes Eastern Cooperative Oncology Group and EGFR epidermal growth factor receptor.
†Race or ethnic group was reported by the patients.
‡Russia was counted as part of Europe, and Turkey was counted as part of Asia.
§Other histologic types included: adenocarcinoma & squamous cell carcinoma, lepidus adenocarcinoma, non-small-cell carcinoma, pleomorphic carcinoma, and unknown.
||One patient in the amivantamab-lazertinib group had both EGFR mutation types (Exon 19 deletion and Exon 21 L858R).

TABLE 12
Efficacy Endpoints*

| | Amivantamab- | | | |
|---|---|---|---|---|
| | lazertinib | Osimertinib | Treatment Effect | |
| Endpoint | (n = 429) | (n = 429) | (95% CI) | P Value |
| Progression- | | | | |
| free survival† | | | | |
| Median (95% | 23.7 (19.1- | 16.6 (14.8- | Hazard ratio, 0.70 | <0.001 |
| CI)-mo | 27.7) | 18.5) | (0.58-0.85) | |
| Patients (95% | | | | |
| CI)-% | | | | |
| At 12 | 73 (69-77) | 65 (60-69) | | |
| months | | | | |
| At 18 | 60 (55-64) | 48 (43-53) | | |
| months | | | | |
| At 24 | 48 (42-54) | 34 (28-39) | | |
| months | | | | |
| Overall | | | | |
| survival | | | | |
| Median (95% | NE | NE | Hazard ratio, 0.80 | 0.11 |
| CI)-mo | | | (0.61-1.05) | |
| Patients (95% | | | | |
| CI)-% | | | | |
| At 12 | 90 (86-92) | 88 (85-91) | | |
| months | | | | |
| At 18 | 82 (78-85) | 79 (75-83) | | |
| months | | | | |
| At 24 | 74 (69-78) | 69 (64-74) | | |
| months | | | | |
| Objective | | | | |
| response | | | | |
| rate‡ | | | | |
| Patients (95% | 86 (83-89) | 85 (81-88) | | |
| CI)-% | | | | |
| Response | | | | |
| duration‡ | | | | |
| Median (95% | 25.8 (20.1-NE) | 16.8 (14.8- | | |
| CI)§-mo | | 18.5) | | |

*The efficacy population included all the patients who had undergone randomization. NE denotes not estimable.
†Progression-free survival (the primary outcome) was assessed by blinded independent central review.
‡The objective response (complete or partial response) and response duration was assessed by blinded independent central review. Included in the analysis were 421 patients with measurable disease at baseline in the amivantamab-lazertinib group and 414 in the osimertinib group.
§Among confirmed responders.

At the time of interim survival analysis, the median overall survival could not be estimated in either group, with 214 deaths reported in the amivantamab-lazertinib and osimertinib groups of the 390 deaths anticipated during the trial period (FIG. 5 and Table 12).

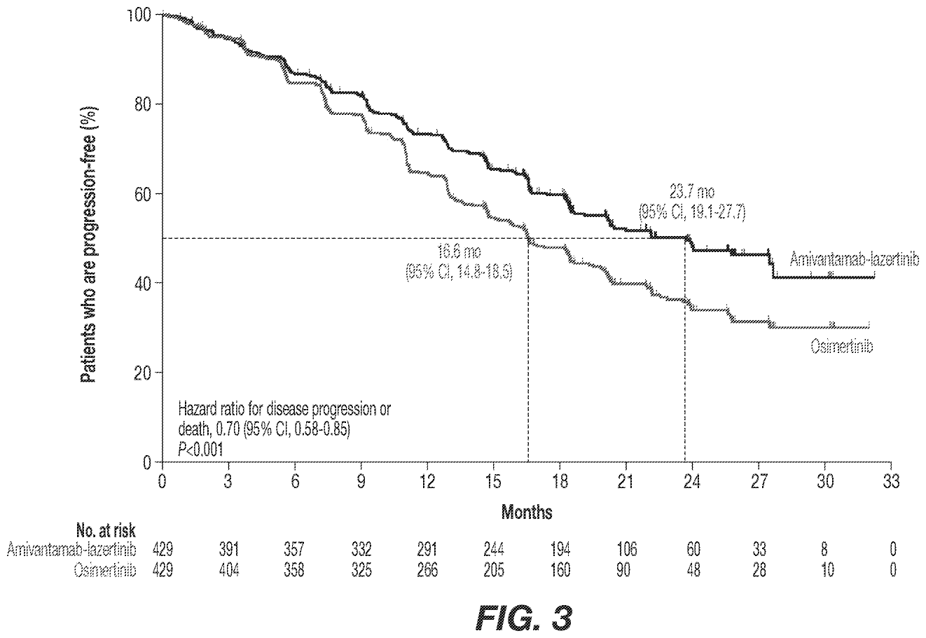

The median progression-free survival by blinded independent central review was 23.7 months (95% CI, 19.1 to 27.7) in the amivantamab-lazertinib group and 16.6 months (95% CI, 14.8 to 18.5) in the osimertinib group (FIG. 3 and Table 12).

TABLE 13
Adverse Events*

| | Amivantamab- | | | |
|---|---|---|---|---|
| | lazertinib | | Osimertinib | |
| Event-no. (%) | (n = 421) | | (n = 428) | |
| Any event | 421 (100) | | 425 (99) | |
| Grade ≥3 | 316 (75) | | 183 (43) | |
| Any serious event | 205 (49) | | 143 (33) | |
| Any event resulting in death | 34 (8) | | 31 (7) | |
| Any event leading to: | | | | |
| Interruptions of any study agent | 350 (83) | | 165 (39) | |
| Reductions of any study agent | 249 (59) | | 23 (5) | |
| Discontinuations of any study | 147 (35) | | 58 (14) | |
| agent | | | | |
| Adverse events reported in ≥15% of | All | Grade | All | Grade |
| patients in either group† | | ≥3 | | ≥3 |
| Paronychia | 288 (68) | 46 (11) | 121 (28) | 2 (0.5) |
| Infusion-related reaction | 265 (63) | 27 (6) | 0 | 0 |
| Rash | 260 (62) | 65 (15) | 131 (31) | 3 (1) |
| Hypoalbuminemia | 204 (48) | 22 (5) | 26 (6) | 0 |
| Increased alanine aminotransferase | 152 (36) | 21 (5) | 57 (13) | 8 (2) |
| Peripheral edema | 150 (36) | 8 (2) | 24 (6) | 0 |
| Constipation | 123 (29) | 0 | 55 (13) | 0 |
| Diarrhea | 123 (29) | 9 (2) | 190 (44) | 3 (1) |
| Dermatitis acneiform | 122 (29) | 35 (8) | 55 (13) | 0 |
| Stomatitis | 122 (29) | 5 (1) | 90 (21) | 1 (0.2) |
| Increased aspartate | 121 (29) | 14 (3) | 58 (14) | 5 (1) |
| aminotransferase | | | | |
| COVID-19 | 111 (26) | 8 (2) | 103 (24) | 9 (2) |
| Decreased appetite | 103 (24) | 4 (1) | 76 (18) | 6 (1) |
| Pruritus | 99 (24) | 2 (0.5) | 73 (17) | 1 (0.2) |
| Anemia | 96 (23) | 16 (4) | 91 (21) | 7 (2) |
| Nausea | 90 (21) | 5 (1) | 58 (14) | 1 (0.2) |
| Hypocalcemia | 88 (21) | 9 (2) | 35 (8) | 0 |
| Asthenia | 78 (19) | 12 (3) | 46 (11) | 4 (1) |
| Pulmonary embolism | 73 (17) | 35 (8) | 20 (5) | 10 (2) |
| Fatigue | 70 (17) | 6 (1) | 42 (10) | 4 (1) |
| Muscle spasms | 70 (17) | 2 (0.5) | 32 (7) | 0 |
| Dry skin | 67 (16) | 1 (0.2) | 60 (14) | 1 (0.2) |
| Thrombocytopenia | 66 (16) | 1 (0.2) | 84 (20) | 5 (1) |
| Cough | 65 (15) | 0 | 88 (21) | 0 |
| Pain in extremity | 64 (15) | 1 (0.2) | 22 (5) | 0 |
| Dyspnea | 51 (12) | 6 (1) | 68 (16) | 17 (4) |
| Leukopenia | 26 (6) | 1 (0.2) | 66 (15) | 0 |

*The safety population included all the patients who had undergone randomization and received at least one dose of any trial treatment.
†Events in this category are listed according to decreasing incidence in the amivantamab-lazertinib group.

Most patients in the trial had at least one adverse event (Table 13). Infusion-related reactions occurred in 63% of patients treated with amivantamab-lazertinib (Table 13), with the majority occurring on cycle 1 day 1. In the amivantamab-lazertinib group, adverse events leading to dose interruption of any trial agent were reported in 350 patients (83%), reductions of any agent in 249 patients (59%), and discontinuations of any agent in 147 patients (35%); the numbers in the osimertinib group were 165 (39%), 23 (5%), 58 (14%), respectively (Table 13).

TABLE 14
Representativeness of Study Participants

| Category | Details |
|---|---|
| Disease under investigation | EGFR-mutated (Ex19del or L858R) NSCLC |
| Special considerations related | |
| to: | |
| Sex | EGFR-mutated NSCLC affects more females (43.7%) |
| | than males (24.0%).¹ |
| Age | The median age from a meta-analysis* of 456 NSCLC |
| | studies was 63 years.¹ |
| Race or ethnic group | Overall, the highest prevalence of EGFR-mutated NSCLC |
| | was in Asian populations (38.8%), followed by 17.4% in |
| | Caucasians, 17.2% in African Americans, and 27.0% in |
| | mixed populations.¹ In a prospective analysis of 1482 |
| | Asian patients, the EGFR mutation frequency with |
| | advanced NSCLC (adenocarcinoma histology) was |
| | 51.4%, where the most commonly detected mutations |
| | were Ex19del (alone: 22.1%; in combination with others: |
| | 24.3%) and L858R (alone: 20.9%; in combination with |
| | others: 22.9%).² |
| Geography | Similar to the estimates above by race, the highest |
| | prevalence of EGFR-mutated NSCLC was in the Asia- |
| | Pacific region (point estimates vary from 38.4%* to |
| | 47%*) and the lowest in Oceania (12%+).^{1,3} Based on |
| | model estimates‡, Ex19del is most prominent in South |
| | America (66.8%) and least prominent in Oceania (40.3%), |
| | while L858R is most prominent in Asia (41.1%) and least |
| | prominent in South America (27.7%).⁴ |
| Other considerations | EGFR-mutated NSCLC is more commonly observed in |
| | patients who are never smokers (49.3% vs 21.5% for |
| | those with history of smoking*) and with the |
| | adenocarcinoma histology type (38.0% vs 11.7% for non- |
| | adenocarcinoma*).¹ The prevalence of EGFR Ex19del |
| | and L858R are similar among NSCLC disease stages (I, |
| | 34.0%; II, 29.9%; III, 33.8%; IV, 37.5%).* |
| Overall representativeness of | The demographic and epidemiologic characteristics of the |
| this trial | MARIPOSA patient population were generally consistent |
| | with the current data for patients with EGFR-mutated |
| | NSCLC.¹ There were more females than males, most |
| | patients did not have a history of smoking, and the highest |
| | prevalence of EGFR-mutated NSCLC was observed in |
| | Asian and White patients. |

EGFR, epidermal growth factor receptor;
Ex19del, exon 19 deletion;
NSCLC, non-small cell lung cancer.
*Obtained from a systematic review and meta-analysis of 456 studies of patients with NSCLC.
†Pooled prevalence of EGFR mutations in patients with NSCLC; data obtained from a systematic review of 87 studies.
‡Values obtained from linear mixed-effects models fitted to EGFR mutation endpoints using logistic transformation, assuming a binomial distribution, of 74 studies with EGFR Ex19del and L858R NSCLC.

Most patients were female, Asian or White, and had never smoked, which is representative of the EGFR-mutated NSCLC population (Table 14).

TABLE 15
First Subsequent Systemic Therapy*

| | Amivantamab- | |
|---|---|---|
| | lazertinib | Osimertinib |
| Therapy-no. of patients (%) | (n = 429) | (n = 429) |
| With investigator-assessed disease progression | 116 | 171 |
| and discontinued all randomized treatment | | |
| Any first subsequent therapy | 78 (67) | 124 (73) |
| EGFR-targeted or TKI-based regimen | 37 (32) | 39 (23) |
| EGFR TKI only | 30 (26) | 29 (17) |
| Osimertinib | 19 (16) | 19 (11) |
| Gefitinib | 4 (3) | 2 (1) |
| Furmonertinib | 4 (3) | 1 (0.6) |
| Aumolertinib | 2 (2) | 2 (1) |
| Icotinib | 1 (1) | 1 (0.6) |
| Catequentinib | 1 (1) | 0 |
| Afatinib | 0 | 2 (1) |
| Erlotinib | 0 | 1 (0.6) |
| Afatinib + osimertinib | 0 | 1 (0.6) |
| EGFR-targeted or TKI-based combination | 7 (6) | 10 (6) |
| Osimertinib + carboplatin + pemetrexed | 1 (1) | 1 (0.6) |
| Osimertinib + cisplatin + pemetrexed | 1 (1) | 1 (0.6) |
| Afatinib + bevacizumab + carboplatin + | 1 (1) | 0 |
| pemetrexed | | |
| Aumolertinib + bevacizumab | 1 (1) | 0 |
| Erlotinib + carboplatin + pemetrexed | 1 (1) | 0 |
| Osimertinib + bevacizumab + carboplatin + | 1 (1) | 0 |
| pemetrexed | | |
| Aumolertinib + HS-10241 | 0 | 2 (1) |
| Amivantamab + lazertinib | 0 | 1 (0.6) |
| Aumolertinib + carboplatin + etoposide | 0 | 1 (0.6) |
| Icotinib + carboplatin + etoposide | 0 | 1 (0.6) |
| Osimertinib + capmatinib | 0 | 1 (0.6) |
| Osimertinib + tepotinib | 0 | 1 (0.6) |
| Chemotherapy or immunotherapy-based | 40 (34) | 81 (47) |
| regimens | | |
| Chemotherapy alone | 30 (26) | 50 (29) |
| Carboplatin + pemetrexed | 16 (14) | 26 (15) |
| Carboplatin + paclitaxel | 6 (5) | 10 (6) |
| Carboplatin + etoposide | 2 (2) | 0 |
| Cisplatin + etoposide | 2 (2) | 0 |
| Cisplatin + pemetrexed | 1 (1) | 9 (5) |
| Pemetrexed | 1 (1) | 1 (0.6) |
| Carboplatin | 1 (1) | 0 |
| Carboplatin + vinorelbine | 1 (1) | 0 |
| Carboplatin + cisplatin + gemcitabine + | 0 | 1 (0.6) |
| paclitaxel | | |
| Carboplatin + docetaxel | 0 | 1 (0.6) |
| Cisplatin + docetaxel | 0 | 1 (0.6) |
| Vinorelbine | 0 | 1 (0.6) |
| Chemotherapy plus VEGF inhibitors | 2 (2) | 9 (5) |
| Carboplatin + pemetrexed + bevacizumab | 2 (2) | 3 (2) |
| Cisplatin + pemetrexed + bevacizumab | 0 | 3 (2) |
| Pemetrexed + bevacizumab | 0 | 2 (1) |
| Carboplatin + cisplatin + pemetrexed + | 0 | 1 (0.6) |
| bevacizumab | | |
| Chemotherapy plus immunotherapy | 1 (1) | 8 (5) |
| Cisplatin + paclitaxel + serplulimab | 1 (1) | 0 |
| Carboplatin + pemetrexed + pembrolizumab | 0 | 4 (2) |
| Carboplatin + pemetrexed + camrelizumab | 0 | 2 (1) |
| Carboplatin + paclitaxel + atezolizumab | 0 | 1 (0.6) |
| Carboplatin + durvalumab + etoposide | 0 | 1 (0.6) |
| Chemotherapy combined with VEGF inhibitors | 6 (5) | 14 (8) |
| and immunotherapy | | |
| Carboplatin + paclitaxel + bevacizumab + | 3 (3) | 9 (5) |
| atezolizumab | | |
| Carboplatin + pemetrexed + bevacizumab + | 1 (1) | 1 (0.6) |
| sintilimab | | |
| Carboplatin + paclitaxel + bevacizumab | 1 (1) | 1 (0.6) |
| Cisplatin + paclitaxel + bevacizumab + | 1 (1) | 0 |
| atezolizumab | | |
| Carboplatin + pemetrexed + bevacizumab + | 0 | 1 (0.6) |
| atezolizumab | | |
| Cisplatin + pemetrexed + bevacizumab + | 0 | 1 (0.6) |
| sintilimab | | |
| Pemetrexed + bevacizumab + atezolizumab | 0 | 1 (0.6) |
| VEGF inhibitor only | | |
| Bevacizumab | 1 (1) | 0 |
| Other | | |
| Amivantamab monotherapy | 0 | 1 (0.6) |
| Antibody drug conjugate | 0 | 1 (0.6) |
| Herbal | 1 (1) | 1 (0.6) |
| Antineoplastic agents | 0 | 2 (1) |

*The efficacy population included all the patients who had undergone randomization (n = 429 for amivantamab-lazertinib and n = 429 for osimertinib); shown here are percentages among the 116 patients in the amivantamab-lazertinib group and 171 patients in the osimertinib group who had investigator-assessed disease progression on and discontinued their randomized treatment.

At a median follow-up of 22.0 months, the median treatment duration was 18.5 months (range, 0.2 to 31.4) with amivantamab-lazertinib and 18.0 months (range, 0.2 to 32.7) with osimertinib. At data cutoff, the assigned treatment was still being administered in 230 patients (55%) in the amivantamab-lazertinib group and 213 (50%) in the osimertinib group. The most common reasons for treatment discontinuation of the amivantamab-lazertinib combination versus osimertinib was progressive disease (86 [20%] versus 154 [36%], respectively) and adverse events (86 [20%] versus 50 [12%], respectively). Among patients with disease progression who discontinued their randomized treatment, 67% in the amivantamab-lazertinib group and 73% in the osimertinib group started a first subsequent therapy (Table 15).

TABLE 16
Response Endpoints*

| | Amivantamab- | |
|---|---|---|
| | lazertinib | Osimertinib |
| Endpoint | (n = 429) | (n = 429) |
| Objective response† | | |
| Patients (95% CI) including all | 86 (83-89) | 85 (81-88) |
| responders-% | | |
| Patients (95% CI) including only | 80 (76-84) | 76 (71-80) |
| confirmed responders-% | | |
| Best overall response†-no. (%) | | |
| Complete response‡ | 29 (7) | 15 (4) |
| Partial response‡ | 334 (79) | 335 (81) |
| Stable disease | 30 (7) | 42 (10) |
| Progressive disease | 7 (2) | 11 (3) |
| Not evaluable | 21 (5) | 11 (3) |
| Response duration† | | |
| Median (95% CI) among all | 24.0 (18.5-NE) | 14.9 (12.9-17.5) |
| responders-mo | | |
| Median (95% CI) among | 25.8 (20.1-NE) | 16.8 (14.8-18.5) |
| confirmed responders-mo | | |

*The efficacy population included all the patients who had undergone randomization. NE denotes not estimable.
†The objective response (complete or partial response) and response duration was assessed by blinded independent central review. Included in the analysis were 421 patients with measurable disease at baseline in the amivantamab-lazertinib group and 414 in the osimertinib group.
‡Includes all responders.

TABLE 17
Treatment-emergent Serious Adverse Events Occurring
in At Least 1% of Patients*

| | Amivantamab- | |
|---|---|---|
| | lazertinib | Osimertinib |
| Event-no. (%) | (n = 421) | (n = 428) |
| Pulmonary embolism | 26 (6) | 10 (2) |
| Pneumonia | 17 (4) | 21 (5) |
| Deep vein thrombosis | 12 (3) | 2 (0.5) |
| COVID-19 | 10 (2) | 10 (2) |
| Pleural effusion | 9 (2) | 17 (4) |
| Infusion related reaction | 9 (2) | 0 |
| Alanine aminotransferase increased | 8 (2) | 6 (1) |
| Pneumonitis | 7 (2) | 8 (2) |
| Rash | 7 (2) | 0 |
| Respiratory failure | 6 (1) | 2 (0.5) |
| Interstitial lung disease | 5 (1) | 5 (1) |
| Hyponatremia | 5 (1) | 4 (1) |
| Hypoalbuminemia | 5 (1) | 0 |
| Dyspnea | 4 (1) | 11 (3) |
| Pericardial effusion | 4 (1) | 5 (1) |
| Cardiac failure | 1 (0.2) | 5 (1) |

*The safety population included all the patients who had undergone randomization and received at least one dose of any trial treatment.
*Events in this category are listed according to decreasing incidence in the amivantamab-lazertinib group.

Adverse events of grade 3 or higher were reported by 75% of patients treated with amivantamab-lazertinib and 43% with osimertinib. The most common grade 3 or higher (at least 10% in either group) adverse events were paronychia and rash. Serious adverse events were reported in 49% of patients treated with amivantamab-lazertinib and 33% with osimertinib (Table 17).

TABLE 18
Incidence and Median Time to Onset of Select Adverse Events of
Interest*

| | Amivantamab- | Osimertinib |
|---|---|---|
| Event-no. (%) | lazertinib (n = 421) | (n = 428) |
| Rash† | | |
| No. of patients (%) | 373 (89) | 210 (49) |
| No. of days to first onset-median (range) | 14 (1-556) | 29 (1-799) |
| No. of events, any grade | 1447 | 238 |
| Duration of event-median no. of days | 31 (1-663) | 51 (1-625) |
| (range) | | |
| Venous thromboembolism‡ | | |
| No. of patients (%) | 157 (37) | 39 (9) |
| No. of days to first onset-median (range) | 84 (6-777) | 194 (10-675) |
| Within first 4 months-no. of patients (%) | 97 of 157 (62) | 13 of 39 (33) |
| Interstitial lung disease* | | |
| No. of patients (%) | 13 (3) | 13 (3) |
| No. of days to first onset-median (range) | 110 (29-443) | 115 (9-512) |
| No. of events, any grade | 14 | 15 |
| Duration of event-median no. of days | 18 (1-191) | 20 (1-189) |
| (range) | | |

*The safety population included all the patients who had undergone randomization and received at least one dose of any trial treatment.
†Included the following preferred terms: rash, dermatitis acneiform, folliculitis, rash maculopapular, skin lesion, acne, erythema, rash pustular, dermatitis, rash pruritic, rash papular, rash erythematous, rash macular, dermatitis infected, erythema multiforme, papule, drug eruption, rash follicular, rash vesicular, skin exfoliation, epidermolysis.
‡Included the following preferred terms: pulmonary embolism, deep vein thrombosis, venous thrombosis limb, thrombosis, venous thrombosis, superficial vein thrombosis, thrombophlebitis, embolism, embolism venous, jugular vein thrombosis, pulmonary infarction, axillary vein thrombosis, portal vein thrombosis, post thrombotic syndrome, sigmoid sinus thrombosis, superior sagittal sinus thrombosis, vena cava thrombosis, pelvic venous thrombosis, pulmonary thrombosis.
§Included the following preferred terms: pneumonitis and interstitial lung disease.

TABLE 19
Venous Thromboembolism Events and Anticoagulation Use*

| | Amivantamab- | Osimertinib |
|---|---|---|
| Event-no. (%) | lazertinib (n = 421) | (n = 428) |
| Any venous thromboembolism event | 157 (37) | 39 (9) |
| Grade 1 | 5 (1) | 0 |
| Grade 2 | 105 (25) | 24 (6) |
| Grade 3 | 43 (10) | 12 (3) |
| Grade 4 | 2 (0.5) | 1 (0.2) |
| Grade 5 | 2 (0.5) | 2 (0.5) |
| Any venous thromboembolism event | 2 (0.5) | 2 (0.5) |
| leading to death | | |
| Any venous thromboembolism event | 12 (3) | 2 (0.5) |
| leading to discontinuation of any | | |
| agent | | |
| Venous thromboembolism events† | | |
| Pulmonary embolism | 73 (17) | 20 (5) |
| Deep vein thrombosis | 61 (14) | 11 (3) |
| Venous thrombosis limb | 17 (4) | 1 (0.2) |
| Thrombosis | 9 (2) | 1 (0.2) |
| Venous thrombosis | 8 (2) | 1 (0.2) |
| Superficial vein thrombosis | 6 (1) | 0 |
| Thrombophlebitis | 6 (1) | 4 (1) |
| Embolism | 4 (1) | 3 (1) |
| Embolism venous | 4 (1) | 1 (0.2) |
| Jugular vein thrombosis | 3 (1) | 0 |
| Pulmonary infarction | 2 (0.5) | 0 |
| Axillary vein thrombosis | 1 (0.2) | 0 |
| Portal vein thrombosis | 1 (0.2) | 0 |
| Post thrombotic syndrome | 1 (0.2) | 0 |
| Sigmoid sinus thrombosis | 1 (0.2) | 0 |
| Superior sagittal sinus thrombosis | 1 (0.2) | 0 |
| Vena cava thrombosis | 1 (0.2) | 0 |
| Pelvic venous thrombosis | 0 | 1 (0.2) |
| Pulmonary thrombosis | 0 | 1 (0.2) |
| Superior vena cava syndrome | 0 | 1 (0.2) |
| Anticoagulation use | | |
| At baseline | 21 (5) | 23 (5) |
| Concomitant use | 199 (47) | 81 (19) |
| First bleeding event while on | 34 (8) | 14 (3) |
| anticoagulants | | |
| Recurrent venous | 8 (2) | 0 |
| thromboembolism event while on | | |
| anticoagulants | | |

*The safety population included all the patients who had undergone randomization and received at least one dose of any trial treatment.
†Events in this category are listed according to decreasing incidence in the amivantamab-lazertinib group.

Venous thromboembolic (VTE) events were reported in 37% of patients in the amivantamab-lazertinib group and 9% in the osimertinib group (Table 18), with the most common being pulmonary embolism and deep-vein thrombosis (Table 19). Notably, 5% of patients received anticoagulation at baseline in both groups. At time of first VTE, few patients were receiving anticoagulation (1% for amivantamab-lazertinib and 0% for osimertinib). Of VTE events, 62% occurred in the first 4 months of treatment in the amivantamab-lazertinib group versus 33% in the osimertinib group. Interstitial lung disease/pneumonitis were reported by 3% of patients, with 1% of grade 3 or higher, in both groups.

TABLE 20
Treatment-emergent Adverse Events Leading to Treatment
Interruptions, Reductions, and Discontinuations*

| | Amivantamab- | |
|---|---|---|
| | lazertinib | Osimertinib |
| Event-no. (%) | (n = 421) | (n = 428) |
| Any event leading to interruptions of | 350 (83) | 165 (39) |
| any study agent | | |
| Most common events leading to | | |
| interruptions of any study agent† | | |
| Rash | 104 (25) | 4 (1) |
| Paronychia | 91 (22) | 4 (1) |
| COVID-19 | 63 (15) | 36 (8) |
| Dermatitis acneiform | 50 (12) | 1 (0.2) |
| Increased alanine aminotransferase | 30 (7) | 12 (3) |
| Hypoalbuminemia | 25 (6) | 0 |
| Increased aspartate | 23 (5) | 11 (3) |
| aminotransferase | | |
| Pulmonary embolism | 20 (5) | 9 (2) |
| Peripheral edema | 20 (5) | 0 |
| Diarrhea | 19 (5) | 6 (1) |
| Deep vein thrombosis | 19 (5) | 2 (0.5) |
| Asthenia | 18 (4) | 1 (0.2) |
| Pneumonia | 14 (3) | 18 (4) |
| Increased γ-glutamyltransferase | 13 (3) | 5 (1) |
| Stomatitis | 13 (3) | 4 (1) |
| Any event leading to dose reductions | 249 (59) | 23 (5) |
| of any study agent | | |
| Most common events leading to dose | | |
| reductions of any study agent‡ | | |
| Rash | 84 (20) | 2 (0.5) |
| Paronychia | 80 (19) | 1 (0.2) |
| Dermatitis acneiform | 38 (9) | 0 |
| Hypoalbuminemia | 11 (3) | 0 |
| Increased alanine aminotransferase | 8 (2) | 0 |
| Paresthesia | 8 (2) | 0 |
| Peripheral edema | 7 (2) | 0 |
| Folliculitis | 6 (1) | 0 |
| Rash maculo-papular | 6 (1) | 0 |
| Increased aspartate | 5 (1) | 0 |
| aminotransferase | | |
| Mucosal inflammation | 5 (1) | 0 |
| Neuropathy peripheral | 5 (1) | 0 |
| Peripheral sensory neuropathy | 5 (1) | 0 |
| Rash pustular | 5 (1) | 0 |
| Skin lesion | 5 (1) | 0 |
| Stomatitis | 5 (1) | 0 |
| Any event leading to discontinuations | 147 (35) | 58 (14) |
| of any study agent | | |
| Most common events leading to | | |
| discontinuations of any study agent‡ | | |
| Infusion related reaction | 19 (5) | 0 |
| Paronychia | 14 (3) | 0 |
| Rash | 11 (3) | 0 |
| Pneumonia | 8 (2) | 3 (1) |
| Pulmonary embolism | 8 (2) | 2 (0.5) |
| Pneumonitis | 7 (2) | 7 (2) |
| Dermatitis acneiform | 6 (1) | 0 |
| Hypoalbuminemia | 6 (1) | 0 |
| Interstitial lung disease | 5 (1) | 4 (1) |
| Asthenia | 5 (1) | 1 (0.2) |
| Peripheral edema | 5 (1) | 0 |

*The safety population included all the patients who had undergone randomization and received at least one dose of any trial treatment.
†Listed are adverse events that were reported in at least 3% of patients in any group.
‡Listed are adverse events that were reported in at least 1% of patients in any group.

The most common adverse events leading to discontinuation of any agent were infusion-related reactions and paronychia (Table 20). Discontinuations of all agents due to treatment-related adverse events was 10% in the amivantamab-lazertinib group and 3% in the osimertinib group.

TABLE 21
Treatment-related Adverse Events*

| | Amivantamab-lazertinib | Osimertinib |
|---|---|---|
| Event-no. (%) | (n = 421) | (n = 428) |
| Any event | 414 (98) | 378 (88) |
| Grade ≥3 | 252 (60) | 59 (14) |
| Any serious event | 97 (23) | 24 (6) |
| Any event resulting in death | 4 (1) | 2 (0.5) |

| | | | | |
|---|---|---|---|---|
| Adverse events reported in ≥15% | All | Grade ≥3 | All | Grade ≥3 |
| of patients in either group† | | | | |
| Paronychia | 285 (68) | 44 (10) | 115 (27) | 2 (0.5) |
| Infusion-related reaction | 265 (63) | 27 (6) | 0 | 0 |
| Rash | 256 (61) | 65 (15) | 121 (28) | 3 (1) |
| Hypoalbuminemia | 164 (39) | 17 (4) | 11 (3) | 0 |
| Increased alanine | 122 (29) | 17 (4) | 38 (9) | 4 (1) |
| aminotransferase | | | | |
| Dermatitis acneiform | 122 (29) | 34 (8) | 54 (13) | 0 |
| Stomatitis | 116 (28) | 4 (1) | 77 (18) | 1 (0.2) |
| Peripheral edema | 112 (27) | 6 (1) | 10 (2) | 0 |
| Diarrhea | 99 (24) | 8 (2) | 149 (35) | 2 (0.5) |
| Increased aspartate | 95 (23) | 9 (2) | 34 (8) | 1 (0.2) |
| aminotransferase | | | | |
| Pruritus | 93 (22) | 2 (0.5) | 63 (15) | 1 (0.2) |
| Decreased appetite | 73 (17) | 2 (0.5) | 40 (9) | 1 (0.2) |
| Nausea | 68 (16) | 2 (0.5) | 32 (7) | 1 (0.2) |
| Dry skin | 65 (15) | 1 (0.2) | 54 (13) | 1 (0.2) |
| Thrombocytopenia | 57 (14) | 1 (0.2) | 75 (18) | 3 (1) |

*The safety population included all the patients who had undergone randomization and received at least one dose of any trial treatment.
†Events in this category are listed according to decreasing incidence in the amivantamab-lazertinib group.

TABLE 22
All Grade 5 Adverse Events*

| | Amivantamab-lazertinib | Osimertinib |
|---|---|---|
| Event-no. (%) | (n = 421) | (n = 428) |
| Pneumonia | 5 (1) | 4 (1) |
| Respiratory failure | 4 (1) | 2 (0.5) |
| Sudden death | 4 (1)† | 1 (0.2) |
| Death | 3 (1) | 2 (0.5) |
| Myocardial infarction | 3 (1)‡ | 0 |
| Pulmonary embolism | 2 (0.5) | 2 (0.5) |
| Septic shock | 2 (0.5) | 1 (0.2) |
| COVID-19 | 1 (0.2) | 3 (1) |
| Pericardial effusion | 1 (0.2) | 1 (0.2) |
| Acinetobacter sepsis | 1 (0.2) | 0 |
| Acute respiratory distress | 1 (0.2) | 0 |
| syndrome | | |
| Arteriosclerosis coronary artery | 1 (0.2) | 0 |
| Cardiopulmonary failure | 1 (0.2) | 0 |
| Cerebral infarction | 1 (0.2) | 0 |
| Circulatory collapse | 1 (0.2) | 0 |
| Coronary artery disease | 1 (0.2)§ | 0 |
| COVID-19 pneumonia | 1 (0.2) | 0 |
| Ischemic cerebral infarction | 1 (0.2) | 0 |
| Myocardial rupture | 1 (0.2) | 0 |
| Pneumonitis | 1 (0.2)§ | 0 |
| Urosepsis | 1 (0.2) | 0 |
| Dyspnea | 0 | 3 (1) |
| Interstitial lung disease | 0 | 2 (0.5)§ |
| Cardiac failure | 0 | 1 (0.2) |
| Cerebral hemorrhage | 0 | 1 (0.2) |
| Cerebrovascular accident | 0 | 1 (0.2) |
| Hemoptysis | 0 | 1 (0.2) |
| Ketoacidosis | 0 | 1 (0.2) |
| Lower respiratory tract infection | 0 | 1 (0.2) |
| Metabolic acidosis | 0 | 1 (0.2) |
| Pleural effusion | 0 | 1 (0.2) |
| Pneumonia aspiration | 0 | 1 (0.2) |
| Respiratory tract infection | 0 | 1 (0.2) |
| Sepsis | 0 | 1 (0.2) |

*The safety population included all the patients who had undergone randomization and received at least one dose of any trial treatment. All grade 5 adverse events correspond to treatment-emergent adverse events leading to death and vice versa.
†One event in the amivantamab-lazertinib group was deemed related to any study treatment by the investigator.
‡Two events in the amivantamab-lazertinib group were deemed related to any study treatment by the investigator.
§Deemed as related to any study treatment by the investigator.

Adverse events leading to death occurred in 34 (8%) patients in the amivantamab-lazertinib group and 31 (7%) in the osimertinib group (Table 22).

The analysis demonstrated a statistically significant and clinically meaningful improvement in PFS in the amivantamab+lazertinib arm, with a 30% reduction in the risk of progression or death compared with osimertinib arm. With a median follow-up of 22.0 months, median PFS in the amivantamab+lazertinib arm was 23.72 months compared to a median PFS of 16.59 months for the osimertinib arm. (HR: 0.70; 95% CI: [0.58, 0.85], p=0.0002). Strong trend in OS favoring the combination of amivantamab and lazertinib compared with osimertinib (HR: 0.80; 95% CI: [0.61, 1.05], p=0.1099). The safety profile of amivantamab is well defined and tolerable, largely consistent with its on-target activity against the EGFR and MET pathways. The safety profile of lazertinib is also well defined and tolerable, and largely consistent with that seen with other 3rd generation EGFR TKI.

### Example 3. Amivantamab Plus Lazertinib Vs Osimertinib in First-Line EGFR-Mutant Advanced Non-Small Cell Lung Cancer (NSCLC) with Biomarkers of High-Risk Disease: a Secondary Analysis from the Phase 3 MARIPOSA Study

MARIPOSA (NCT04487080) is an international Phase 3 randomized study of amivantamab and lazertinib combination therapy versus osimertinib versus lazertinib as first-line treatment in approximately 1000 subjects with EGFR-mutated locally advanced or metastatic NSCLC (Study 73841937NSC3003, also known as NSC3003, and Mariposa).

Amivantamab+lazertinib meaningfully improved PFS, PFS2, and DoR versus osimertinib in MARIPOSA.

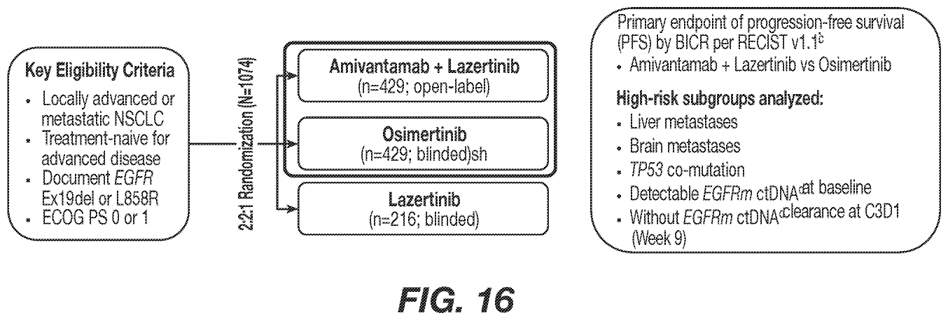

Key Eligibility Criteria: locally advanced or metastatic NSCLC, treatment-naïve for advanced disease, documented EGFR Ex19del or L858R, and ECOG PS 0 or 1. Primary endpoint of progression-free survival (PFS) by BICR per RECIST v1.1c: Amivantamab+Lazertinib vs Osimertinib. High-risk subgroups analyzed: liver metastases, brain metastases, TP53 co-mutation, detectable EGFRm ctDNAd at baseline, and without EGFRm ctDNAd clearance at C3D1 (Week 9) (FIG. 16). Detection of ctDNA and co-mutations were analyzed by next-generation sequencing (NGS) of blood at baseline. Detection and clearance of Ex19del and L858R ctDNA in blood were analyzed with ddPCR at baseline and C3D1.

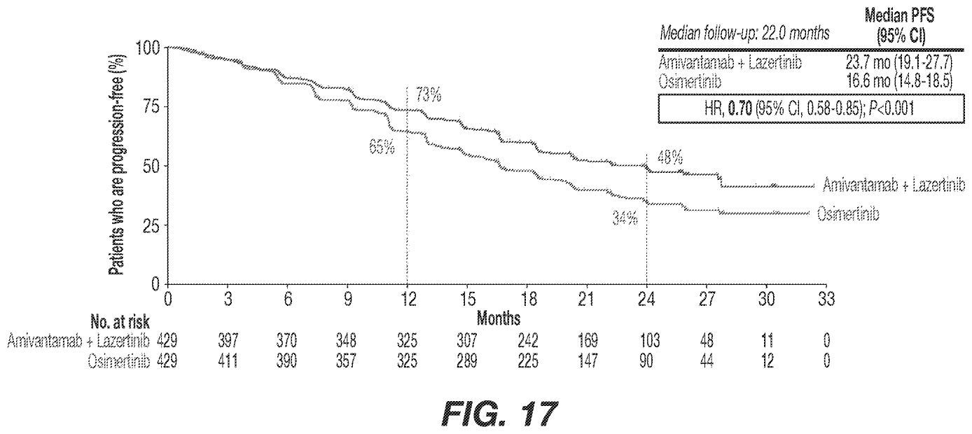

Primary Endpoint: Progression-free Survival by BICR. Amivantamab+Lazertinib reduced the risk of progression or death by 30% and improved median PFS by 7.1 months. (FIG. 17).

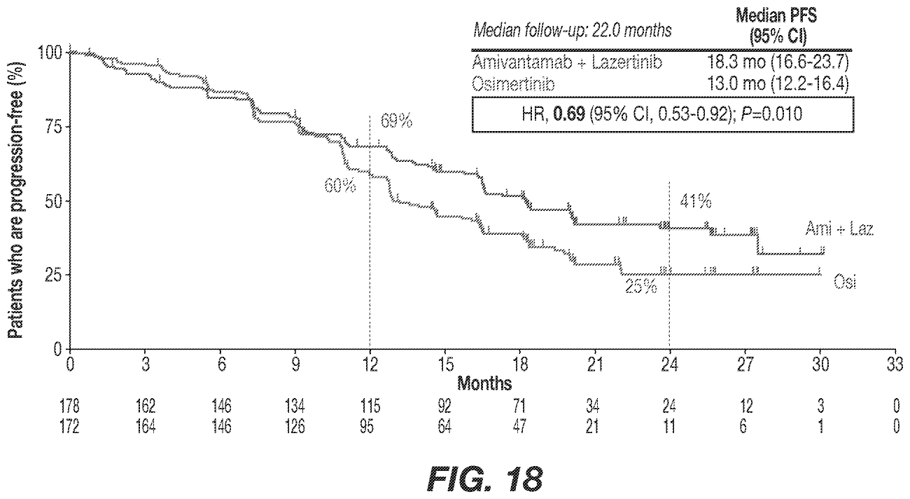

Patients with Brain Metastases: Osimertinib showed a median PFS of 13.0 months among patients with brain metastases at baseline, indicating a poor prognostic subgroup. Among patients with brain metastases at baseline, amivantamab+lazertinib reduced the risk of progression or death by 31% vs Osimertinib. (FIG. 18). Among patients without brain metastases at baseline, amivantamab+lazertinib showed a consistent benefit over osimertinib: Median PFS: 27.5 vs 19.9 months and HR 0.69 (95% CI, 0.53-0.89); P=0.005. (FIG. 18).

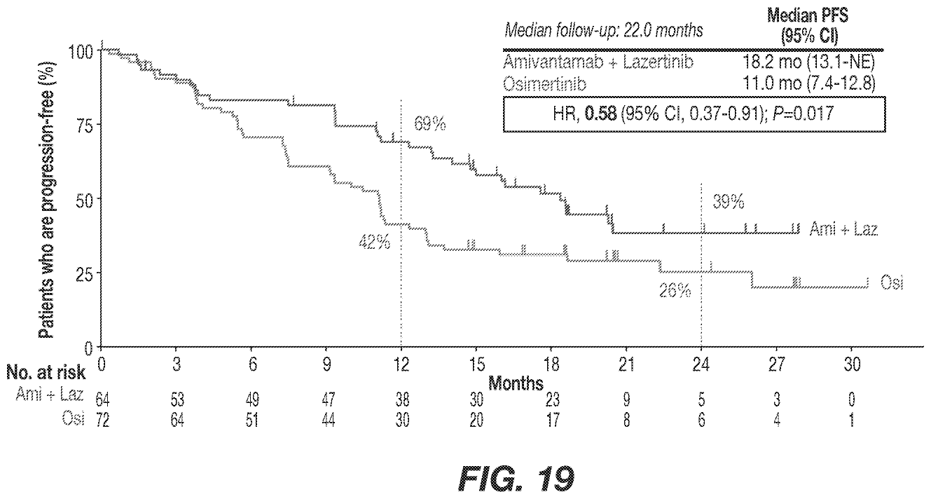

Patients with Liver Metastases: Osimertinib showed a median PFS of 11.0 months among patients with liver metastases at baseline, indicating a poor prognostic subgroup. Among patients with liver metastases at baseline, amivantamab+lazertinib reduced the risk of progression or death by 42% vs Osimertinib. (FIG. 19). Among patients without liver metastases at baseline, amivantamab+lazertinib showed a consistent benefit over osimertinib: Median PFS: 24.0 vs 18.3 months and HR 0.74 (95% CI, 0.60-0.91); P=0.004. (FIG. 19).

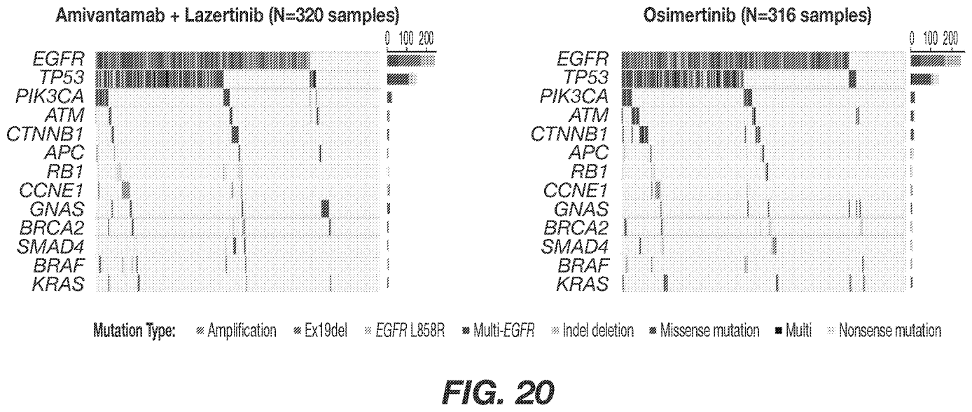

Next-Generation Sequencing (NGS) Circulating Tumor DNA (ctDNA) Pathogenic Mutation Patterns at Baseline: 85% (540/636 samples) had pathogenic alterations detected in ctDNA at baseline by NGS. TP53 co-mutations were observed in 56% from the amivantamab+lazertinib arm and 53% from the osimertinib arm. MET amplification occurred in 1 patient in each arm (neither with high-level amplification). (FIG. 20).

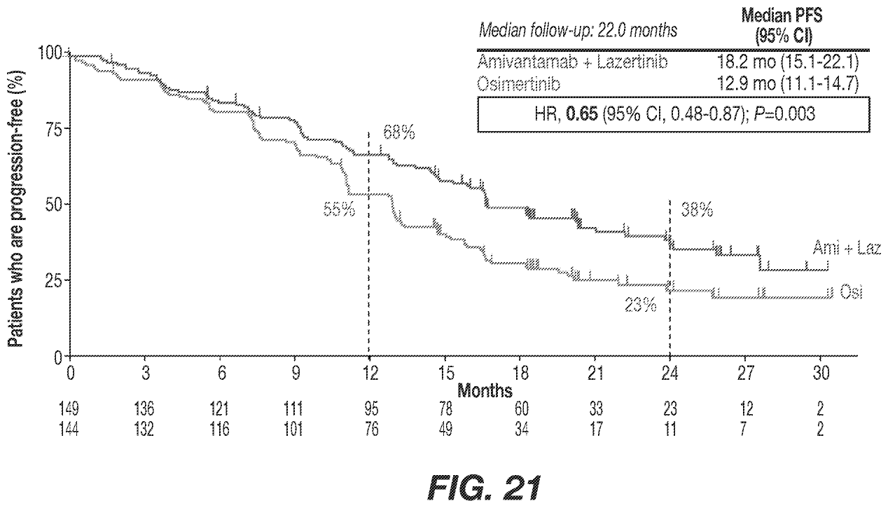

Patients With TP53 Co-Mutations: Osimertinib showed a median PFS of 12.9 months among patients with TP53 co-mutations at baseline, indicating a poor prognostic subgroup. Among patients with TP53 co-mutations at baseline, amivantamab+lazertinib reduced the risk of progression or death by 35% vs Osimertinib. (FIG. 21). Among patients with wild-type TP53 at baseline, amivantamab+lazertinib showed a consistent benefit over osimertinib: Median PFS: 22.1 vs 19.9 months and HR 0.75 (95% CI, 0.52-1.07); P=0.114. (FIG. 21).

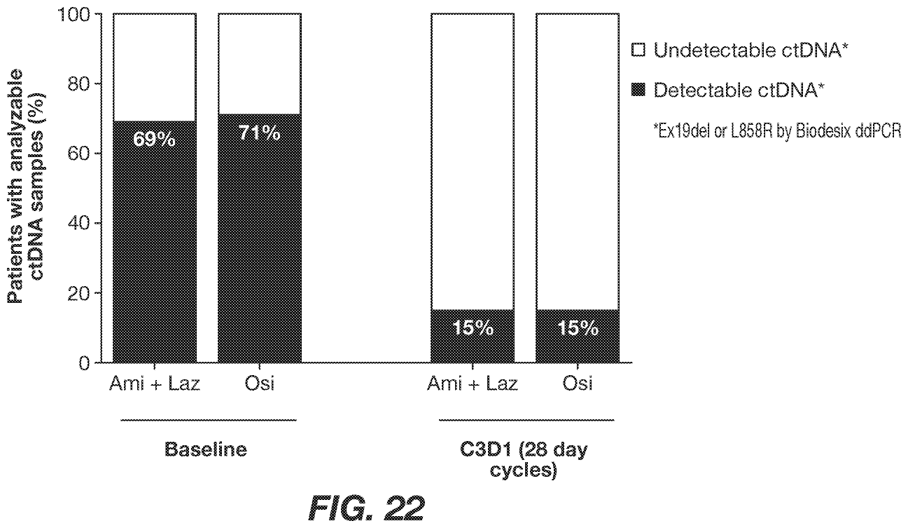

Detectable EGFRm ctDNA at Baseline and On Treatment: Detection and clearance of Ex19del and L858R ctDNA in the blood were analyzed by ddPCR (Ex19del or L858R by Biodesix ddPCR). At baseline, 336 patients in both the amivantamab+lazertinib and osimertinib arms provided analyzable ctDNA samples. Approximately 70% of patients in both arms had detectable EGFRm ctDNA (Ex19del or L858R by Biodesix ddPCR) at baseline. (FIG. 22). 192 patients in the amivantamab+lazertinib arm and 212 in the osimertinib arm had matched samples at baseline and C3D1 (Week 9) (cycles were 28 days). At C3D1 (Week 9) (cycles were 28 days), detectable EGFRm ctDNAa was observed in 15% of these patients in both arms. (FIG. 22).

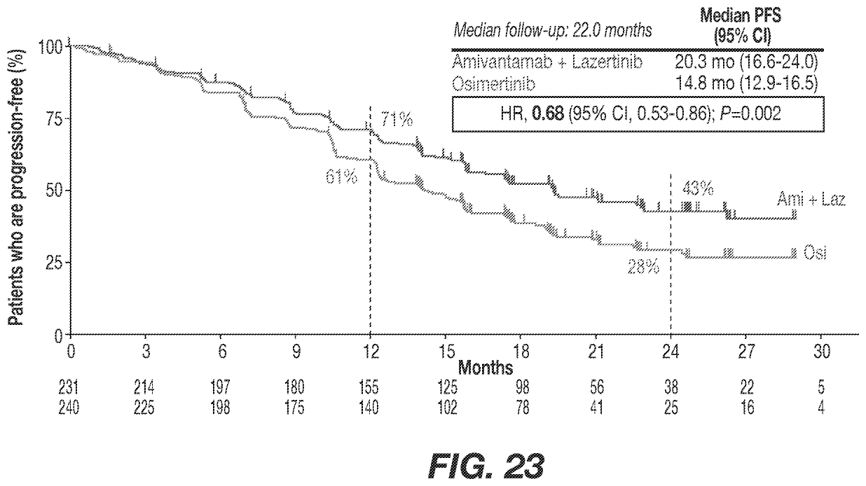

Patients With Detectable Baseline ctDNA (Ex19del or L858R by Biodesix ddPCR): Osimertinib showed a median PFS of 14.8 months among patients with detectable ctDNAa at baseline, indicating a poor prognostic subgroup. Among patients with detectable baseline ctDNA (Ex19del or L858R by Biodesix ddPCR), amivantamab+lazertinib reduced the risk of progression or death by 32% vs Osimertinib. (FIG. 23). Among patients without detectable baseline ctDNA (Ex19del or L858R by Biodesix ddPCR), amivantamab+lazertinib showed a consistent benefit over osimertinib: Median PFS: 27.7 vs 21.9 months and HR 0.72 (95% CI, 0.47-1.10); P=0.132. (FIG. 23). Among patients with detectable baseline ctDNA by Guardant360® NGS, amivantamab+lazertinib showed a consistent benefit over osimertinib (HR, 0.71 [95% CI, 0.57-0.89]; P=0.003). (FIG. 23).

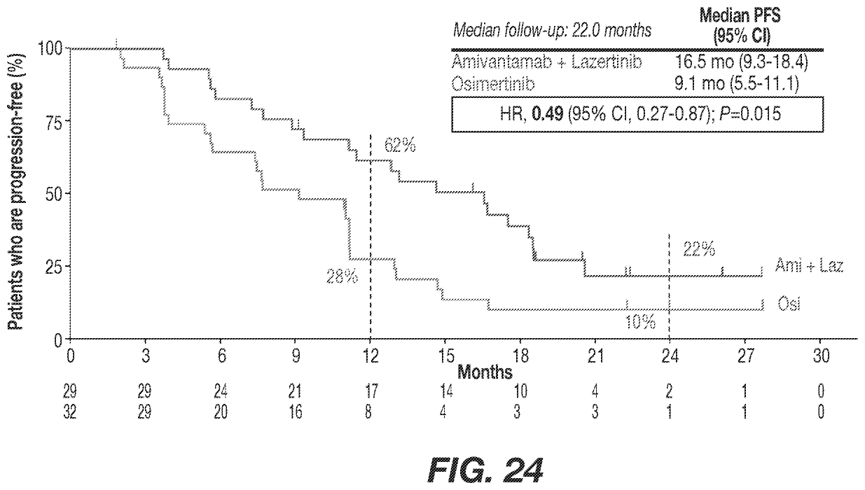

Patients Without Cleared ctDNA at C3D1 (Ex19del or L858R by Biodesix ddPCR. Cycles were 28 days): Osimertinib showed a median PFS of 9.1 months among patients without cleared ctDNA at C3D1 (Ex19del or L858R by Biodesix ddPCR. Cycles were 28 days), indicating a poor prognostic subgroup. Among patients without cleared ctDNA at C3D1 (Ex19del or L858R by Biodesix ddPCR. Cycles were 28 days), amivantamab+lazertinib reduced the risk of progression or death by 51% vs Osimertinib. (FIG. 24). Among patients with cleared ctDNA at C3D1a, amivantamab+lazertinib showed a consistent benefit over osimertinib: Median PFS: 24.0 vs 16.5 months and HR 0.64 (95% CI, 0.48-0.87); P=0.004. (FIG. 24).

PFS for Patients With High-risk Features: In the MARIPOSA study, 89% of patients had at least 1 high-risk feature detected at baseline (Patients with analyzable ctDNA by NGS at baseline were included in this pooled analysis. High-risk features included baseline detectable ctDNA by NGS or baseline metastases of the liver or brain. For patients with detectable ctDNA, it was assumed TP53 co-mutations would be identified if present).

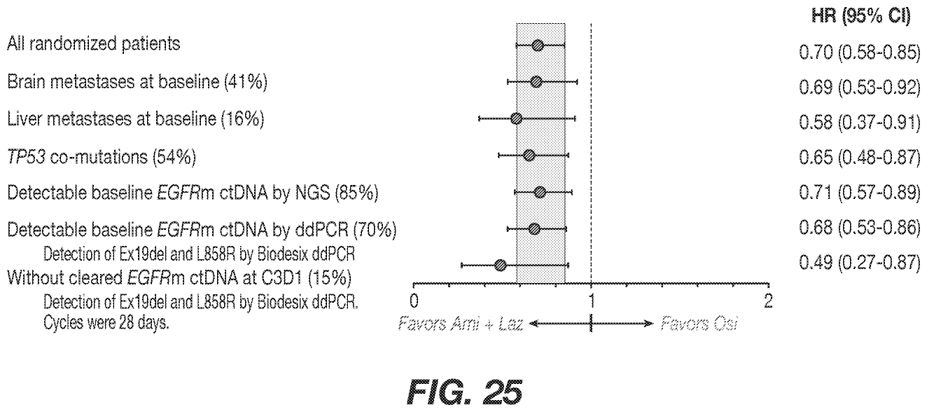

Amivantamab+lazertinib in first line EGFR-mutant (Ex19del/L858R) advanced NSCLC significantly improved PFS vs osimertinib in patients with high-risk features including those:
a.) With baseline brain metastases (HR, 0.69; P=0.010)
b.) With baseline liver metastases (HR, 0.58; P=0.017)
c.) With TP53 co-mutations (HR, 0.65; P=0.003)
d.) With detectable baseline EGFRm ctDNA (Ex19del and L858R by Biodesix ddPCR) (HR, 0.68; P=0.002)
e.) Without EGFRm ctDNA (Ex19del and L858R by Biodesix ddPCR) clearance at C3D1 (Cycles were 28 days) (HR, 0.49; P=0.015) (FIG. 25).

An estimated 89% of patients had at least 1 high-risk feature at baseline (Patients with analyzable ctDNA by NGS at baseline were included in this pooled analysis. High-risk features included baseline detectable ctDNA by NGS or baseline metastases of the liver or brain. For patients with detectable ctDNA, it was assumed TP53 co-mutations would be identified if present)

Among the corresponding subgroups without a high-risk feature, amivantamab+lazertinib showed a consistent PFS benefit over Osimertinib. Amivantamab+Lazertinib effectively overcomes the effect of high-risk features and represents a promising new standard-of-care for patients with EGFR-mutant advanced NSCLC.

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

All patents, applications, publications, test methods, literature, and other materials cited herein are hereby incorporated by reference in their entirety as if physically present in this specification.

1. A method of improving median progression free survival (PFS) in a population of subjects or improving overall survival (OS) in a subject or a population of subjects with locally advanced or metastatic non-small cell lung cancer (NSCLC) harboring one or more epidermal growth factor receptor (EGFR) mutations who are treatment-naïve, the method comprising administering to the subject or the population of subjects a combination therapy comprising:
(i) a therapeutically effective amount of a bispecific anti-EGFR/c-Met antibody, and
(ii) a therapeutically effective amount of lazertinib, or a pharmaceutically acceptable salt or hydrate thereof, and
wherein the improvement in median PFS is relative to median PFS of a reference population of subjects with NSCLC harboring one or more EGFR mutations who are treatment-naive, the reference population having been administered osimertinib or lazertinib without the bispecific anti-EGFR/c-Met antibody; or
wherein the improvement in OS is relative to OS of a reference subject or a reference population of subjects with NSCLC harboring one or more EGFR mutations who are treatment-naive, the reference subject or the reference population of subjects having been administered osimertinib or lazertinib without the bispecific anti-EGFR/c-Met antibody.

2. (canceled)

3. The method of claim 1, wherein the lazertinib, or pharmaceutically acceptable salt or hydrate thereof, is lazertinib mesylate.

4. The method of claim 1, wherein the lazertinib, or pharmaceutically acceptable salt or hydrate thereof, is lazertinib mesylate monohydrate.

5. The method of claim 1, wherein the one or more EGFR mutations comprise one or more exon 19 deletions, or exon 21 L858R substitution, or any combination thereof.

6. The method of claim 1, wherein the one or more EGFR mutations comprise one or more exon 19 deletions.

7. The method of claim 1, wherein the one or more EGFR mutations comprise exon 21 L858R substitution.

8. The method of claim 1, wherein the subject has newly diagnosed, locally advanced or metastatic NSCLC that is not amenable to curative therapy including surgical resection or chemoradiation.

9. The method of claim 8, wherein the curative therapy includes surgical resection or chemoradiation.

10. The method of claim 1, wherein the method comprises administering the lazertinib, or pharmaceutically acceptable salt or hydrate thereof, in an amount of about 80 mg to about 320 mg orally once daily.

11. The method of claim 10, wherein the method comprises administering the lazertinib, or pharmaceutically acceptable salt or hydrate thereof, in an amount of about 240 mg orally once daily.

12. The method of claim 1, wherein the method elicits a clinical response in the subject according to RECIST v1.1 criteria.

13. The method of claim 1, wherein the method achieves a partial response or better in the subject according to RECIST v1.1 criteria.

14. The method of claim 1, wherein the clinical response comprises a median duration of response (DOR) of at least 25 months.

15. The method of claim 1, wherein the subject is progression-free after at least 11 months.

16. The method of claim 1, wherein the subject is progression-free after at least 23 months.

17. The method of claim 1, wherein the method achieves a PFS rate of 87% at 6 months, 73% at 12 months, 60% at 18 months, 48% at 24 months, and 41% at 30 months in a population of the treatment naïve subjects diagnosed with locally advanced or metastatic NSCLC harboring one or more epidermal growth factor receptor (EGFR) mutations.

18. The method of claim 1, wherein the bispecific anti-EGFR/c-Met antibody comprises a first domain that specifically binds EGFR and a second domain that specifically binds c-Met, wherein the first domain comprises a heavy chain complementarity determining region 1 (HCDR1) comprising SEQ ID NO: 1, a HCDR2 comprising SEQ ID NO: 2, a HCDR3 comprising SEQ ID NO: 3, a light chain complementarity determining region 1 (LCDR1) comprising SEQ ID NO: 4, a LCDR2 comprising SEQ ID NO: 5, and a LCDR3 comprising SEQ ID NO: 6, and wherein the second domain that binds c-Met comprises a HCDR1 comprising SEQ ID NO: 7, a HCDR2 comprising SEQ ID NO: 8, a HCDR3 comprising SEQ ID NO: 9, a LCDR1 comprising SEQ ID NO: 10, a LCDR2 comprising SEQ ID NO: 11, and a LCDR3 comprising SEQ ID NO: 12.

19. The method of claim 18, wherein the first domain that specifically binds EGFR comprises a heavy chain variable region (VH) comprising SEQ ID NO: 13 and a light chain variable region (VL) comprising SEQ ID NO: 14, and the second domain that specifically binds c-Met comprises a VH comprising SEQ ID NO: 15 and a VL comprising SEQ ID NO: 16.

20. The method of claim 19, wherein the bispecific anti-EGFR/c-Met antibody is an IgG1 isotype.

21. The method of claim 19, wherein the bispecific anti-EGFR/c-Met antibody comprises a first heavy chain (HC1) comprising SEQ ID NO: 17, a first light chain (LC1) comprising SEQ ID NO: 18, a second heavy chain (HC2) comprising SEQ ID NO: 19, and a second light chain (LC2) comprising SEQ ID NO: 20.

22. The method of claim 19, wherein the bispecific anti-EGFR/c-Met antibody comprises a biantennary glycan structure with a fucose content of about between 1% to about 15%.

23. The method of claim 1, wherein the bispecific anti-EGFR/c-Met antibody is administered intravenously to the subject.

24. The method of claim 23, wherein the bispecific anti-EGFR/c-Met antibody is administered at a dose of between about 140 mg to about 2240 mg.

25. The method of claim 24, wherein the bispecific anti-EGFR/c-Met antibody is administered at a dose of about 700 mg, about 750 mg, about 800 mg, about 850 mg, about 900 mg, about 950 mg, about 1000 mg, about 1050 mg, about 1100 mg, about 1150 mg, about 1200 mg, about 1250 mg, about 1300 mg, about 1350 mg, about 1400 mg, about 1575 mg, about 1600 mg, about 2100 mg, or about 2240 mg.

26. The method of claim 25, wherein the bispecific anti-EGFR/c-Met antibody is administered at a dose of 1050 mg if the subject has a body weight of less than 80 kg.

27. The method of claim 25, wherein the bispecific anti-EGFR/c-Met antibody is administered at a dose of 1400 mg if the subject has a body weight of greater than or equal to 80 kg.

28. The method of claim 1, wherein the bispecific anti-EGFR/c-Met antibody is administered subcutaneously or intradermally to the subject.

29. The method of claim 28, wherein the bispecific anti-EGFR/c-Met antibody is administered subcutaneously or intradermally at a dose sufficient to achieve a therapeutic effect in the subject.

30. The method of claim 1, wherein the bispecific anti-EGFR/c-Met antibody is administered twice a week, once a week, once in two weeks, once in three weeks, or once in four weeks.

31. The method of claim 1, wherein the subject or population of subjects has a baseline brain metastasis, baseline liver metastasis, TP53 co-mutation, detectable baseline EGFRm ctDNA, or is without EGFRm ctDNA clearance at C3D1.

32. The method of claim 1, wherein the method improves the median progression free survival (PFS) in a population of subjects.

33. The method of claim 1, wherein the method improves the overall survival (OS) in a subject or a population of subjects.


SEQUENCE LISTING (20 sequences)

SEQ ID NO: 1 - protein, 5 aa, synthetic construct
TYGMH

SEQ ID NO: 2 - protein, 17 aa, synthetic construct
VIWDDGSYKYYGDSVKG

SEQ ID NO: 3 - protein, 16 aa, synthetic construct
DGITMVRGVMKDYFDY

SEQ ID NO: 4 - protein, 11 aa, synthetic construct
RASQDISSALV

SEQ ID NO: 5 - protein, 7 aa, synthetic construct
DASSLES

SEQ ID NO: 6 - protein, 9 aa, synthetic construct
QQFNSYPLT

SEQ ID NO: 7 - protein, 5 aa, synthetic construct
SYGIS

SEQ ID NO: 8 - protein, 17 aa, synthetic construct
WISAYNGYTNYAQKLQG

SEQ ID NO: 9 - protein, 10 aa, synthetic construct
DLRGTNYFDY

SEQ ID NO: 10 - protein, 11 aa, synthetic construct
RASQGISNWLA

SEQ ID NO: 11 - protein, 7 aa, synthetic construct
AASSLLS

SEQ ID NO: 12 - protein, 9 aa, synthetic construct
QQANSFPIT

SEQ ID NO: 13 - protein, 125 aa, synthetic construct [sequence omitted]

SEQ ID NO: 14 - protein, 107 aa, synthetic construct [sequence omitted]

SEQ ID NO: 15 - protein, 119 aa, synthetic construct
QVQLVQSGAEVKKPGASVKVSCETSGYTFTSYGISWVRQAPGHGLEWMGWISAYNGYTNYAQKLQGRVTMTTDTSTSTAYMELRSLRSDDTAVYYCARDLRGTNYFDYWGQGTLVTVSS

SEQ ID NO: 16 - protein, 107 aa, synthetic construct
DIQMTQSPSSVSASVGDRVTITCRASQGISNWLAWFQHKPGKAPKLLIYAASSLLSGVPSRFSGSGSGTDFTLTISSLQPEDFATYYCQQANSFPITFGQGTRLEIK

SEQ ID NO: 17 - protein, 455 aa, synthetic construct
QVQLVESGGGVVQPGRSLRLSCAASGFTFSTYGMHWVRQAPGKGLEWVAVIWDDGSYKYYGDSVKGRFTISRDNSKNTLYLQMNSLRAEDTAVYYCARDGITMVRGVMKDYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFLLYSKLTVDKSRWQQGNVFSCSVMHEALHNHYTQKSLSLSPGK

SEQ ID NO: 18 - protein, 214 aa, synthetic construct
AIQLTQSPSSLSASVGDRVTITCRASQDISSALVWYQQKPGKAPKLLIYDASSLESGVPSRFSGSESGTDFTLTISSLQPEDFATYYCQQFNSYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 19 - protein, 449 aa, synthetic construct
QVQLVQSGAEVKKPGASVKVSCETSGYTFTSYGISWVRQAPGHGLEWMGWISAYNGYTNYAQKLQGRVTMTTDTSTSTAYMELRSLRSDDTAVYYCARDLRGTNYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSRLTVDKSRWQQGNVFSCSVMHEALHNHYTQKSLSLSPGK

SEQ ID NO: 20 - protein, 214 aa, synthetic construct
DIQMTQSPSSVSASVGDRVTITCRASQGISNWLAWFQHKPGKAPKLLIYAASSLLSGVPSRFSGSGSGTDFTLTISSLQPEDFATYYCQQANSFPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC